(19) United States
(12) Patent Application Publication
(10) Pub. No.: US 2024/0360236 A1
(43) Pub. Date: Oct. 31, 2024

(54) METHODS FOR TARGETED IMMUNOTHERAPY OF ACUTE MYELOID LEUKEMIA (AML)

(71) Applicant: HACKENSACK MERIDIAN HEALTH, INC., Edison, NJ (US)

(72) Inventor: Johannes ZAKRZEWSKI, New York, NY (US)

(73) Assignee: HACKENSACK MERIDIAN HEALTH, INC., Edison, NJ (US)

(21) Appl. No.: 18/637,595

(22) Filed: Apr. 17, 2024

Related U.S. Application Data
(60) Provisional application No. 63/460,255, filed on Apr. 18, 2023.

(51) Int. Cl. C07K 16/28; A61K 39/00; A61P 35/02

(52) U.S. Cl. CPC C07K 16/2878; A61P 35/02; C07K 16/2809; A61K 2039/505; C07K 2317/31; C07K 2317/732

(57) ABSTRACT

The present disclosure provides a method for treating an eligible subject with acute myeloid leukemia including high-risk disease features comprising administering to the subject post-consolidation a targeted immunotherapy comprising an immunotherapeutic agent specifically targeting B cell maturation antigen.

### CROSS-REFERENCE TO RELATED APPLICATIONS

This application claims the benefit of priority to U.S. provisional application 63/460,255, filed Apr. 18, 2023, the contents of which are incorporated by reference herein in their entirety.

### FIELD OF THE INVENTION

The present disclosure relates to therapies for treating cancer, and more particularly to targeted immunotherapies for treating a cancer where B cell maturation antigen is expressed by blast cells.

### BACKGROUND OF THE INVENTION

### I. Immune Mechanisms Implicated in the Body's Response to Cancer

### The Immune Response

The human immune system is a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

### Immunological Synapses and Immune Cell Activation

Immune responses are initiated by the interaction between antigen presenting cells (APCs), such as dendritic cells (DCs), with responder cells, such as T cells, via a tight cellular contact interface called the immunological synapse. The immunological synapse is a highly organized subcellular structure that provides a platform for the presentation of antigen in major histocompatibility class I and II complexes (MHC class I and II) on the surface of the APC to receptors on the surface of the responder cells. In T cells, these contacts lead to highly polarized membrane trafficking that results in the local release of lytic granules and in the delivery and recycling of T cell receptors at the immunological synapse. Localized trafficking also occurs at the APC side of the synapse, especially in DCs where antigen loaded in MHC class I and II is presented and cytokines are released specifically at the synapse. A functional immunological synapse between DCs and naïve T cells is essential to mount functional T cell responses. [Vergoogen, D R J et al. Biomol Concepts (2016) 7 (1): 17-28].

Not only DCs and T cells, but also other APCs, such as B cells or infected cells, and other effector cells, such as natural killer cells (NKs), form immunological synapses for intercellular communication as well as for the killing of infected or aberrant target cells. [Id., citing Angus, K L and Griffiths, GM. Curr. Opin. Cell Biol. (2013) 25:85-91; Friedl, P. et al. Nat. Rev. Immunol. (2005) 5:53; Xie, J. et al. Immunol. Rev. (2013) 251:65-79]. The structure of the synapse strongly depends on the cell types involved, the presence and strength of antigen recognition and additional co-stimulatory interactions. [Id., citing Friedl, P. et al. Nat. Rev. Immunol. (2005) 5:53; Thauland, T J and Parker, DC. Immunology (2010) 131:466-72; Azar, G A et al. Proc. Natl. Acad. Sci. USA (2010) 107:3675-80].

Immunological synapses can functionally be divided into two categories [Id., citing Gerard, A. et al. Immunol. Rev. (2013) 251:80-96]: (1) primary synapses, which are the cell-cell contacts that result in initial activation of immune cells, such as the synapses between DCs and T cells [Id., citing Rodriguez-Fernandez, J L et al. Sci. Signal (2010) 3: re2], and (2) so-called secondary synapses that result from interactions established after initial priming, such as activated T cells delivering stimulatory signals via, for example, CD40-CD40L interactions to B cells [Id., citing Chaplin, DD. J. Allergy Clin. Immunol. (2010) 125: S3-23]; this category also encompasses the synapses formed between NKs or cytotoxic T cells with their target APC where lytic granules are released to kill the APC [Id., citing Stinchcombe, J C et al. Immunity (2001) 15:751-61]. For both categories, the formation of immunological synapses can trigger intracellular signaling cascades in both the APC and the T cell that lead to reorganization of the cytoskeleton and rerouting of membrane trafficking.

Membrane trafficking. Membrane trafficking plays an important role in T cell effector functions, because it leads to surface display of TCRs and other membrane proteins, recycling of exhausted receptors, and to release of cytokines and chemokines at the immunological synapse. The best understood form of exocytosis at the immunological synapse is the release of cytolytic granules from CD8+ T cells and NKs. Other types of cargo that are delivered and/or recycled at the T cell side of the immunological synapse include cytokines (e.g. IFN-gamma), and membrane receptors (e.g. TCR, ICAM-1) [Id., citing Griffiths, G M et al. J. Cell Biol. (2010) 189:397-406; Angus, KL, Griffiths, GM. Curr. Opin. Cell Biol. (2013) 25:85-91; Xic, J. et al. Immunol. Rev. (2013) 251:65-79; Jo, J H et al. J. Cell Biochem. (2010) 111:1125-37; Finetti, F. and Baldari, CT. Immunol. Rev. (2013) 251:97-112; Das, V. et al. Immunity (2004) 20:577-88; Soares H. et al. J. Exp. Med. (2013) 210:2415-33]. The polarized delivery of these molecules to the immunological synapse allows a more sensitive antigen presentation and/or promotes T cell effector functions, while preventing unwanted activation of other (immune) cells nearby.

Polarized membrane trafficking occurs at the APC side as well. In DCs, MHC class I and II [Id., citing Boes, M. et al. Nature (2002) 418:983-8; Bertho, N. J. Immunol. (2003) 171:5689-96; Boes, M. et al. J. Immunol. (2003) 171:4081-8; Compeer, E B et al. J. Biol. Chem. (2014) 289:520-8] and the costimulatory molecule CD40 [Id., citing Foster, N. et al. J. Immunol. (2012) 189:5632-7] can be locally trafficked to and presented at the immunological synapse. The local release of these molecules improves the efficiency of T cell activation and helps to explain how T cells can detect a few MHC ligands among an abundance of endogenous peptide-bound MHC [Id., citing Xic, J. et al. Immunol. Rev. (2013) 251:65-79]. In addition, IL-12 is also locally released by the DC at the immunological synapse with T cells [Id., citing Pulecio, J. et al. J. Exp. Med. (2010) 207:2719-32]. IL-12 promotes a THI response, enhances the cytolytic activity of CD8+ T cells and induces production of IFN-gamma by T cells. The polarized release of IL-12 also was observed at the immunological synapse between DCs and NKs [Id., citing Borg, C. et al. Blood (2004) 104:3267-75; Barreira da Silva, R. et al. Blood (2011) 118:6487-98].

### NK Cells

NK cells are innate immune cells that display rapid and potent cytolytic activity in response to infected or aberrant cells [Gonzalez, H. et al. Genes & Development (2018) 32:1267-84., citing Cerwenka A, Lanier L L. (2016). Nat Rev Immunol 16:112-123]. NK cells have a wide array of inhibitory and stimulatory receptors on their cell surface that are used for immune surveillance. The inhibitory receptors target cancer cells lacking major histocompatibility class I (MHC-I), marking them for programmed cell death [Id., citing Marcus, A. et al. (2014). Adv Immunol 122:91-128]. In contrast, in healthy cells, the binding of MHC-I molecules to their receptors on NK cells has a profound inhibitory effect on NK cell function [Id., citing Bix, M. et al. (1991). Nature 349:329-331; Liao, N S et al. (1991). Science 253:199-202; Colonna, M. et al. (1992). Proc Natl Acad Sci 89:7983-7985; Karlhofer, F M et al. (1992). Nature 358:66-70; Wagtmann, N. et al. (1995). Immunity 2:439-449; Lanier, LL (2005). J Immunol 174:6565]. NK cells have a well-documented anti-tumor effect [Id., citing Marcus, A. et al. (2014). Adv Immunol 122:91-128; Iannello, A. et al. (2016). Curr Opin Immunol 38:52-58].

The conventional NK (cNK) cell pool consists of a circulating compartment and a tissue-resident compartment in the gut intraepithelial layer and lamina propria layer. [Jiao, Y. et al., Front. Immunol. (2020) 11:282] cNK cells are able to sense pathogens, oncogenesis and tissue damage signals. Activation and turnover of cNK cells rely on the overall signal input of activating signals, inhibitory signals, and exogenous cytokine signals, which further leads to the alteration of specific transcription factors and a group of pro-apoptotic proteins and ultimately determines the fate of cNK cells. [Id., citing Viant C, et al. J Exp Med. (2017) 214:491-510]. Upon activation, cNK cells exert their cytotoxicity function by releasing the pore forming cytolytic protein perforin and the cytotoxic protein granzyme. cNK cells also utilize tumor necrosis factor (TNF)-related apoptosis-inducing ligand (TRAIL) pathways and antibody-dependent cellular cytotoxicity (ADCC) (Id., citing Caligiuri M A. Blood. (2008) 112:461-9). At the same time, cNK cells possess strong cytokine production ability, including TNF, IFN-γ, and granulocyte-macrophage colony-stimulating factor (GM-CSF) (Id., citing Souza-Fonseca-Guimaraes F, et al. J Biol Chem. (2013) 288:10715-21).

cNK activation. The theory of cNK education posits that the threshold of activation of cNKs throughout their development is modulated by adjusting the expression level of their activating receptors and inhibitory receptors. The processes of cNK cell arming (meaning the downregulation of inhibitory receptors that could upregulate the threshold of activation) and cNK cell licensing (meaning the scenario where activating receptors are downregulated to endow cNK cells with increased receptivity to activating signals) ensure the appropriate activation strategy, namely, to limit self-reaction of cNK cells that do not recognize self MHC class I molecules by inhibitory receptors. Generally, educated cNK cells, marked by the elevated expression of the activating receptor DNAM-1, exhibit higher reactivity to missing-self targets with increased degranulation and cytokine production capability [Id., citing Enqvist M, et al. J Immunol. (2015) 194:4518-27]. It has been hypothesized that the gut may be one of the centers for cNK cells to obtain normal function and acquire education. Gain of cytotoxic function of cNK cells is dependent on the priming step by commensal bacteria in a dendritic cell dependent manner [Id., citing Ganal S C, et al. Immunity. (2012) 37:171-86] and commensal lactic acid bacteria are a key regulator in the cross-talk between cNK cells. Lactic acid bacteria activate immature dendritic cells in the gut to produce key cytokines, including IL-12 and IL-15, and to favor the activation and proliferation of cNK cells [Id., citing Rizzello V, et al. BioMed Res Int. (2011) 2011:473097].

### T Cells

T cells are components of the adaptive immune system that act as orchestrators and effectors of immunity. Depending on the immunological context, T cells can acquire functional and effector phenotypes whose activity has direct inflammatory or anti-inflammatory consequences [Gonzalez, H. et al. Genes & Development (2018) 32:1267-84, citing Speiser, D E et al. (2016) Regulatory circuits of T cell function in cancer. Nat Rev Immunol 16:599-611]. As the second most frequent immune cell type found in human tumors besides tumor associated macrophages TAMs), T cells are extensively studied in diverse cancer types [Id., citing Speiser, D E et al. (2016) Nat Rev Immunol 16:599-611; Donadon, M. et al. (2017) J Gastrointest Surg 21:1226-1236]. During the early stages of tumor initiation, if enough immunogenic antigens are produced, naïve T cells will be primed in the draining lymph nodes, followed by their concomitant activation and migration to the tumor microenvironment (TME). From there, they mount a protective effector immune response, eliminating immunogenic cancer cells. Histopathological analyses of human tumors show that tumor-associated T cells extend beyond the invasive edge of the tumor and also predominate in its hypoxic core [Id., citing Halama, N. et al. (2011) Cancer Res 71:5670-5677; Kirilovsky, A. et al. (2016) Int Immunol 28:373-382]. A high level of T-cell infiltration in tumors is associated with a favorable prognosis in melanoma [Id., citing Clemente, C G et al. (1996) Cancer 77:1303-1310] and breast [Id., citing Oldford, S A et al. (2006) Int Immunol 18:1591-1602], lung [Id., citing Dieu-Nosjean, M C et al. (2008) J Clin Oncol 26:4410-4417], ovarian [Id., citing Kusuda, T. et al. (2005) Oncol Rep 13: 1153-1158], colorectal [Id., citing Tosolini, M. et al. (2011) Cancer Res 71:1263-1271, renal [Id., citing Kondo, T. et al. (2006) Cancer Sci 97:780-786], prostate [Id., citing Vesalainen, S. ct al. (1994) Eur J Cancer 30A: 1797-1803, and gastric [Id., citing Ubukata, H. et al. (2010) J Surg Oncol 102:742-747; Fridman, W H et al. (2012) Nat Rev Cancer 12:298-306; Kitamura, T. et al. (2015) Nat Rev Immunol 15:73-86] cancer.

T Cell Activation. T-cell activation is dependent on the interaction of the TCR/CD3 complex with its cognate ligand, a peptide bound in the groove of a class I or class II MHC molecule. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated T lymphocyte is lymphokines (meaning cytokines produced by lymphocytes).

Naïve T cells must initially be activated by dendritic cells. [Yewdell, JW and BP Dolan. Nature (2011) 471 (73340): 581-82].

The TCR-Peptide Loaded MHC (pMHC) Interaction.

TCR recognition of peptide loaded MHC is unparalleled in the diversity of the interacting surfaces. All components of the interaction are inherently extremely diverse: generation of the TCR by somatic recombination allows a theoretical diversity of about 1015-18 different TCRs, of which about 1.5×10⁷ can be found within any individual [Zareic, P. et al. Viral Immunology 33 (3): 179-87 (2020), citing Davis, M M and Bjorkman, PJ. Nature (1988) 334:395-402]; the MHC genes are the most polymorphic genes of the human genome, and the peptide cargo that can be bound by MHC is virtually limitless. Despite this diversity, key structural characteristics have emerged, which are highly conserved across virtually all interactions. The TCR makes contact with both the peptide cargo and the MHC (co-recognition); the TCR binds over the top of the peptide, the peptide is contacted by at least one of the highly variable TCR CDR3 loops, and the TCR binds pMHC with a highly conserved polarity. The TCR a chain sits over the top of the MHCI a2 helix or MHCIIB chain, while the TCR B chain is positioned over the MHCI a helix or MHCII a chain. [Id., citing Rossjohn, J. et al. Annu. Rev. Immunol. (2015) 33:169-200; Rudolph, M G et al. Annu. Rev. Immunol. (2006) 24:419-66].

There are two models that are under debate to understand MHC restriction that address why T cells only see antigens presented by MHC molecules, the germline model and the selection model. The germline model proposes that germline TCR sequences have been selected during evolution to encode TCR structure that efficiently interacts with MHC molecules. The opposing hypothesis comes from the selection model. Selection theories to explain T cell receptor bias for MHC propose that MHC restriction is driven ultimately by constraints imposed on the TCR during positive selection in the thymus and the nature of TCR signaling. [Zareic, P. ct al. Viral Immunology 33 (3): 179-87 (2020), citing Rangarajan, S. and Mariuzza, RA. Cell Mol. Life Sci. (2014) 71:3059-68; Van Laethem, F. et al. Trends Immunol. (2012) 33:437-41]. TCRs themselves do not possess intrinsic signaling capacity. Instead, TCR signaling relies on the delivery of lymphocyte-specific protein tyrosine kinase (Lck) to the CD3 complex when associated with the cytoplasmic tails of CD4 or CD8 co-receptors [Id., citing Turner, J M et al. Cell (1990) 60:755-65]. Thus, the CD4 and CD8 co-receptors act to focus the TCR onto the MHC molecule. This requirement for co-receptor facilitation of signaling prevents development of T cells expressing TCRs that are not specific for MHC, ensuring MHC restriction.

TCR recognition of nonclassical MHC molecules. Unlike classical MHC-I and MHC-II molecules, MHC I-like molecules such as CDI and MRI are monomorphic (meaning having only one form) [Zareie, P. et al. Viral Immunology 33 (3): 179-87 (2020)., citing Mori, L. et al. Annu. Rev. Immunol. (2016) 34:479-510] and present lipid or metabolite antigens, rather than peptides, to TCRs expressed by “nonconventional” T cells such as mucosal associated invariant T (MAIT) cells, natural killer T cells (NKT), and subsets of γδ T cells (reviewed in Godfrey D I, et al. Nat. Immunol. (2012) 13:851-6].

The three key tenets of conventional TCR-pMHC binding: the TCR binding over peptide, TCR co-recognition of both the MHC and the peptide cargo; and the conserved docking polarity of the TCR also have been observed for TCR recognition of nonclassical MHC molecules, including recognition of HLA-E [Id., citing Gherardin, N A et al. Immunity (2016) 44:32-45; Sullivan, L C et al. J. Biol. Chem. (2017) 292:21149-58] and MR-1, which presents metabolites to MAIT cells and to atypical MR1-restricted T cells [Id., citing Corbett, A J et al. Nature (2014) 509:361-65; Eckle, S B et al. J. Exp. Med. (2014) 211:1585-1600; Gherardin, NA ct al. Immunity (2016) 44:32-45; Gherardin, N A et al. J. Immunol. (2018) 201:2862; Patel, O. et al. Nat. Comun. (2013) 4:2142].

CD4+T cells. CD4+T cells play a central role in orchestrating host immune responses against cancer and infectious diseases as well as in autoimmunity [Wang, R-F. Trends in Immunol. (2001) 22 (50): 269-76, citing Paradoll, D M and Topalian, SL. Curr. Opin. Immuno. (1998) 10:588-94; O'Garra, A. et al. (1997) Curr. Opin. Immunol. 9:872-83; Kalams, S A and Walker, BD. J. Exp. Med. (1998) 188:2199-2204; Zajac, A J et al. Curr. Opin. Immuno. (1998) 10:444-49].

When interacting with CD4+T cells, DCs may induce their differentiation into different T helper (TH) subsets [Patente, T A, et al., Frontiers Immunol. (2019) doi.org/10.3389/fimmu.2018.03176., citing Iwasaki A, Medzhitov R. Nat Immunol. (2015) 16:343-353] such as THI [Amsen D, et al. Cell (2004) 117:515-26; Constant S, et al. J Exp Med (1995) 182:1591-6; Hosken N A, et al. J Exp Med. (1995) 182:1579-84; Kadowaki N. Allergol Int. (2007) 56:193-9; Mackawa Y, et al. Immunity (2003) 19:549-59; Pulendran B, et al. Proc Natl Acad Sci USA. (1999) 96:1036-41, TH2 [Id., citing Constant S, et al. J Exp Med (1995) 182:1591-6, Hosken N A, et al. J Exp Med. (1995) 182:1579-84, Jenkins S J, P. et al. J Immunol. (2007) 179:3515-23, Soumelis V, et al. Nat Immunol. (2002) 3:673-680], TH17 [Id., citing Bailey S L, Nat Immunol. (2007) 8:172-80; Iezzi G, et al. Proc Natl Acad Sci USA. (2009) 106:876-81; Huang G, et al. Cell Mol Immunol. (2012) 9:287-95], or other CD4+T cell subtypes [Id., citing Levings M K, et al. Blood (2005) 105:1162-9]. T cell differentiation in each subtype is a complex phenomenon that can be influenced by the cytokines in the DC tissue of origin [Id., citing Rescigno M. Immunol Rev. (2014) 260:118-28], their maturation state [Id., citing Reis e Sousa C. Nature Rev Immunol. (2006) 6:476-83] and cause of tissue imbalance [Id., citing Vega-Ramos J, et al. Curr Opin Pharmacol. (2014) 17:64-70].

FIG. 1 schematically depicts the role of CD4+ T cells in modulating immune responses against cancer. As shown in FIG. 1, CD4+T cells play a central role in regulating the host immune responses through the following mechanisms. (a) CD4+T cells provide crucial help in the priming of CD8+T cells via activation of antigen presenting cells (APCs). (b) CD4+T cells secrete cytokines required for maintaining CD8+T-cell function and proliferation. (c) CD4+T cells can inhibit tumor growth directly or indirectly. (d) CD4+T cells provide help for B-cell activation. Thus, both CD8+ T cells and antibody production require the crucial help of CD4+T cells. Taken from Wang, R-F. Trends in Immunol. (2001) 22 (50): 269-76.

CD4+T cells can be divided into Thelper 1 (TH1) and Thelper 2 (TH2) cells based on their cytokine secretion profile. [Wang, R-F. Trends in Immunol. (2001) 22 (50): 269-76, citing Morel, PA and Criss, TP. Crit. Rev. Immunol. (1998) 18:275-303]. TH2 cells activate B cells to become antibody-secreting plasma cells. CD4+ T_{H}I cells help prime CD8+T cell responses [Id., citing Toes, R E et al. J. Exp. Med. (1999) 189:753-6]. Several studies have demonstrated that CD40-CD40 ligand (CD40L) interactions between DCs and CD4+T cells activate DCs for effective priming and activation of CD8+T cells. [Id., citing Schoenberger, S P et al. Nature (1998) 393:473-4; Bennett, S R et al. Nature (1998) 393:478-80; Ridge, J P et al. Nature (1998) 393:474-78]. CD4+T cells recognize an antigen presented by professional APCs, such as DCs, and, in turn activate antigen-bearing DCs [Id., citing Banchereau, J. and Steinman, RM. Nature (1998) 392:245-52]. Once activated, DCs become competent to prime cytotoxic lymphocytes (CTLs) that recognize an MHC class I-restricted determinant on the same APC. Thus, activation of APCs by CD4+T cells through antigen-specific recognition and CD40-CD40L engagement is essential to prime CD8+T cells. [Id., citing Schoenberger, S P et al. Nature (1998) 393:473-4; Bennett, S R et al. Nature (1998) 393:478-80; Ridge, J P et al. Nature (1998) 393:474-78]. In addition, CD4+ T cells are essential in the maintenance of CD8+T cell effector functions by secreting cytokines such as IL-2 required for CD8+T cell growth and proliferation. [Id., citing Rosenberg, S A et al.] Nature Med. (1998) 4:321-7; Greenberg, PD. Adv. Immunol. (1991) 49:281-355].

Most tumors express MHC class I, but not MHC class II molecules. CD4+T cells recognize peptides presented by MHC class II molecules. on the cell surface of APCs. The formation of MHC class II peptide complexes on the cell surface is a complicated multistep process that favors presentation of antigens derived from exogenous proteins. Assembly of the MHC class II α and β chains, along with the associated invariant chain (Ii), begins in the endoplasmic reticulum (ER). [Id., citing Germain, RN. Cell (1994) 76:287-99; Cresswell, P. Cell (1996) 84:505-7]. Ii association prevents an antigenic peptide from binding to the αβ dimers and stabilizes the αβ complexes. Ii contains an endosome-targeting sequence at the N-terminus, and a class II-associated invariant-chain peptide (CLIP) between amino acids 81 and 104. This targeting sequence in the cytoplasmic tail of Ii is responsible for the transport of nonameric (αβIi) 3 complexes from the ER to intracellular compartments with endosomal/lysosomal characteristics and, ultimately, to acidic endosomal and lysosomal-like structures called MHC class II compartments (MIIC) [Id., citing Germain, RN. MHC-dependent antigen processing and peptide presentation providing ligands for T lymphocyte activation. Cell (1994) 76:287-99; Cresswell, P. Cell (1996) 84:505-7]. HLA-DM molecules in this compartment facilitate dissociation of residual Ii peptide (CLIP) from the peptide-binding grooves of the mHC class II molecules and replacement with antigenic peptides. [Id., citing Cresswell, P. Cell (1996) 84:505-7]. Thus, MHC class II antigen processing and presentation requires at least five genes-DRa, DRB, Ii, DMA and DMB- and the specialized MIIC compartments.

CD8+T cells. CD8+ T cells are the most prominent anti-tumor cells. Upon priming and activation by APCs, the CD8+T cells differentiate into cytotoxic T lymphocytes (CTLs) and, through the exocytosis of perforin- and granzyme-containing granules, exert an efficient anti-tumoral attack, resulting in the direct destruction of target cells [Gonzalez, H. et al. Genes & Development (2018) 32:1267-84, citing Hanson, H L et al. (2000) Immunity 13:265-276; Matsushita, H. et al. (2012) Nature 482:400-404]. Meanwhile, the CD4+T helper 1 (TH1)-mediated anti-tumoral response-through secretion of high amounts of proinflammatory cytokines such as IL-2, TNF-α, and IFN-γ-promotes not only T-cell priming and activation and CTL cytotoxicity but also the anti-tumoral activity of macrophages and NK cells and an overall increase in the presentation of tumor antigens [Id., citing Kalams S A, Walker B D. (1998) J Exp Med 188:2199-2204; Pardoll D M, Topalian S L. (1998). Curr Opin Immunol 10:588-594; Shankaran, V. et al. (2001) Nature 410:1107-1111]. The presence of tumor-infiltrating CD8+T cells and THI cytokines in tumors correlates with a favorable prognosis in terms of overall survival and a disease-free survival in many malignancies [Fridman, W H et al. (2012). Nat Rev Cancer 12:298-306).

Preclinical investigations in patients and mouse models suggest that cancer cells exploit the immunosuppressive properties of T cells while impairing the effector functions of anti-tumor T cells, such as their ability to infiltrate tumors and their survival, proliferation, and cytotoxicity [Id., citing Grivennikov, S I et al. (2010) Cell 140:883-899]. The antigen-dependent nature of the effector T cells implies that the effectiveness of the anti-tumor T-cell immune response depends on both the ability of the tumor antigen to induce an immune response (immunogenic) and the presence—or absence—of inhibitory signals that can impair the T cells' functions [Id., citing Speiser, D E et al. (2016) Nat Rev Immunol 16:599-611]. Accordingly, it is widely accepted that, in a T-cell-dependent process, most neoplastic cells expressing highly immunogenic antigens will be recognized and killed during the early stages of tumor development [Id., citing Matsushita, H. et al. (2012) Nature 482:400-404]. The less immunogenic cancer cells escape the immune control of T cells and survive, a process termed “cancer immune editing” [Id., citing Teng, M W et al. (2015) J Clin Invest 125:3338-3346]. The final outcome is that the surviving cancer cells adopt an immune-resistant phenotype. In parallel, during tumor development, cancer cells evolve mechanisms that mimic peripheral tolerance and are able to prevent the local cytotoxic response of effector T cells as well as those of other cells, such as tumor-associated macrophages (TAMs), NK cells, and tumor-associated neutrophils (TANs) [Id., citing Palucka A K, Coussens L M. (2016) The basis of oncoimmunology. Cell 164:1233-47]. TANs are engaged into the tumor microenvironment by cytokines and chemokines, and can be distinguished according to their activation and cytokine status and effects on tumor cell growing in N1 and N2 TANs. NI TANs exert an antitumor activity, by direct or indirect cytotoxicity. [Masucci, M T et al. Front Oncol. 9:1146].

Immune checkpoints. During immune homeostasis, a crucial mechanism of peripheral tolerance is the regulation of effector T-cell response via immune checkpoints on CTLs and activated CD4+T cells to protect tissue from inflammatory damage. The two better described checkpoint molecules CTLA-4 and PD-1 act as negative regulators of T-cell function and have been associated with immune evasion in cancer [Gonzalez, H. et al. Genes & Development (2018) 32:1267-84., citing Pardoll, DM (2012) Nat Rev Cancer 12:252-264]. The involvement of CTLA-4 signaling in cancer has been described in melanoma ([Id., citing Bouwhuis, M G et al. (2010) Cancer Immunol Immunother 59:303-312] and lung ([Id., citing Khaghanzadeh, N. et al. (2010) Cancer Genet Cytogenet 196:171-174], breast ([Id., citing Erfani N. et al. Cancer Genet. Cytogenet. (2006) 165 (2): 114-20], gastric [Id., citing Hadinia, A. et al. (2007) J Gastroenterol Hepatol 22:2283-2287], and colorectal ([Id., citing Hadinia, A. et al. (2007) J Gastroenterol Hepatol 22:2283-2287; Dilmec, F. et al. (2008). Int J Immunogenet 35:317-321] cancer. Furthermore, the engagement of PDI with its coreceptor, PDL-1 (expressed by other immune cells, mesenchymal cells, vascular cells, and cancer cells), results in the down-regulation of T-cell activity, which inhibits their anti-tumor activities such as T-cell migration, proliferation, secretion of cytotoxic mediators, and restriction of cell killing ([Id., citing Topalian, S L et al. (2015) Cancer Cell 27:450-461]. The use of immune checkpoint inhibitors such as anti-PDI (e.g., lambrolizumab, pembrolizumab and nivolumab), anti-PD-L1 (MPDL3280A), and anti-CTLA4 (ipilimumab) has had success enhancing the effector anti-tumor response in different malignancies ([Id., citing Gotwals, P. et al. (2017). Nat Rev Cancer 17:286-301], especially in melanoma and lung cancer ([Id., citing Hamid, O. et al. (2013) N Engl J Med 369:134-144; Herbst, R S et al. (2014). Nature 515:563-567; Topalian, S L et al. (2015) Cancer Cell 27:450-461].

As the tumor grows and the TME changes, new antigens are produced, and the ability of the immune system to prime new repertoires of T cells and direct them toward the tumor changes, thus altering the efficacy of tumor containment. As the immune system functions to stall tumor growth, cancer cells and the TME simultaneously suppress anti-tumor function by engaging immune checkpoints and the recruitment of regulatory CD4+T cells (Tregs). Tregs are responsible for suppressing the priming, activation, and cytotoxicity of other effector immune cells, such as THI CD4 T cells, CTLs, macrophages, NK cells, and neutrophils ([Id., citing Ward-Hartstonge K A, Kemp R A. (2017). Clin Transl Immunology 6: e154]. The Treg-mediated immunosuppression is orchestrated by contact-dependent mechanisms such as the expression of PDL-1, LAG-3, CD39/73, CTLA4, or PD1, with the latter two even enhancing suppressive activity ([Id., citing Walker L S, Sansom D M. (2015) Trends Immunol 36:63-70], and by contact-independent mechanisms, which involve the sequestration of IL-2 and production of immune-suppressive molecules such as IL-10, TGF-β, prostaglandin E2, adenosine, and galectin-1 [Id., citing Francisco, L M et al. (2009). J Exp Med 206:3015-3029; Campbell, DJ (2015) Eur J Immunol 195:2507-2513]. In squamous cell carcinoma, for example, the inhibition of focal adhesion kinase (FAK)—a cell contact-independent mechanism—results in CCL5 secretion by cancer cells that induces the recruitment of Tregs to the tumor site, where they suppress the cytotoxic anti-tumor CD8+T cells ([Id., citing Serrels, A. et al. (2015) Cell 163:160-173]. In breast and lung adenocarcinoma, for example, Tregs suppress T-cell activation and the anti-tumor immune response in tumor-associated tertiary structures. Specific Treg depletion results in tumor cell death and increased production of IFN-γ ([Id., citing Bos, P D et al. (2013) J Exp Med 210:2435-2466; Joshi, N S et al. (2015) Immunity 43:579-590]. Infiltration of Tregs in breast cancer has been correlated with worse patient outcome [Id., citing Allaoui, R. et al. (2017) Cancer Biomark 20:395-409].

In metastasis, cytotoxic T lymphocytes (CTLs) exert an anti-metastatic effect in bone metastasis [Id., citing Bidwell, B N et al. (2012) Nat Med 18:1224-1231], while prospective analyses of lung and breast cancer patients established an opposite correlation between the level of circulating cancer cells and T cells in peripheral blood [Id., citing Mego, M et al. (2016) Circulating tumor cells (CTC) are associated with defects in adaptive immunity in patients with inflammatory breast cancer. J Cancer 7:1095-1104; Sun, W W et al. (2017) Onco Targets Ther 10:2413-2424].

These data extend to clinical trials reporting the therapeutic efficacy of immune checkpoint inhibition in metastatic carcinomas [Id., citing Di Giacomo, A M et al. (2012) Lancet Oncol 13:879-886; Queirolo, P. et al. (2014) J Neurooncol 118:109-116; Motzer, R J et al. (2015) N Engl J Med 373:1803-1813; Furudate, S. et al. (2016) Case Rep Oncol 9:644-649; Goldberg, S B et al. (2016) Lancet Oncol 17:976-983; Pai-Scherf, L. et al. (2017) Oncologist 22:1392-1399]. Checkpoint inhibitors are significantly effective in treating brain metastatic tumors from melanoma and lung cancer [Id., citing Queirolo, P. et al. (2014). J Neurooncol 118:109-116; Goldberg, S B et al. (2016) Lancet Oncol 17:976-983; Di Giacomo, A M et al. (2017) Cytokine Growth Factor Rev 36:33-38]. Recent evidence suggests that the effectiveness of checkpoint inhibition in melanoma brain metastasis depends on extracranial disease and peripheral activation of CD8+T cells [Id., citing Taggart, D. et al. (2018) Proc Natl Acad Sci 115: E1540-E1549]. On the other hand, a high level of circulating Tregs has been associated with a higher risk of metastasis in non-small lung carcinoma patients. [Id., citing Erfani, N. et al. (2012) Lung Cancer 77:306-311]. Similar associations have been described in breast cancer [Id., citing Metelli, A. et al. (2016) Cancer Res 76:7106-7117], colorectal carcinoma metastasis [Id., citing Wang, Q. et al. (2014) Cell Immunol 287:100-105, and hepatocellular carcinoma [Id., citing Ye, L Y et al. (2016) Cancer Res 76:818-830].

### B Cells

B Cell Activation. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). The soluble product of an activated B lymphocyte is immunoglobulins (antibodies).

Upon activation in the germinal centers in lymphoid organs, B cells expressing high-affinity antibodies differentiate into antibody-secreting plasma cells and memory B cells that mediate humoral immunity against pathogens [Gonzalez, H. et al. Genes & Development (2018) 32:1267-84, citing De Silva N S, Klein U. (2015) Nat Rev Immunol 15:137-148]. Although the presence of B cells in the TME has been described in different carcinomas (including melanoma and breast, ovarian, and prostate cancer, among others) [Id., citing Chin, Y. et al. (1992) Anticancer Res 12:1463-1466; Yang, C. et al. (2013) PLOS One 8: c54029; Woo, J R et al. (2014) J Transl Med 12:30; Pylayeva-Gupta, Y. et al. (2016) Cancer Discov 6:247-255], the role of B cells in cancer progression is much less understood than that of T cells. Accumulating evidence indicates that B cells promote and support tumor growth; for example, using a transgenic mouse model of epithelial carcinogenesis, Coussens and colleagues [Id., citing de Visser, K E et al. (2005) Cancer Cell 7:411-423] demonstrated that the lack of mature B cells decreases tumor progression. The adoptive transfer of B cells restores chronic inflammation, angiogenesis, and tumor growth. Different mechanisms have been described to explain the pro-tumor role of B cells, from immunosuppression via secretion of IL-10 [Id., citing Schioppa, T. et al. (2011) Proc Natl Acad Sci 108:10662-10667 and TGFβ [Id., citing Olkhanud, P B et al. (2011) Cancer Res 71:3505-3515] to direct stimulation of tumor cell proliferation by B-cell-derived IL-35 in human pancreatic neoplasia and Kras-driven pancreatic neoplasms in mice [Id., citing Pylayeva-Gupta, Y. et al. (2016) Cancer Discov 6:247-255]. Also, by deposition of immunoglobulins in the TME, B cells indirectly stimulate angiogenesis and chronic inflammation by activating myeloid cells via FcRy (Andreu, P. et al. (2010) Cancer Cell 17:121-134).

### Memory T Cells

The vast majority of human memory T cells reside in tissue sites, including lymphoid tissues, intestines, lungs and skin. By the end of puberty, lymphoid tissues, mucosal sites and the skin are populated predominantly by memory T cells, which persist throughout adult life and represent the most abundant lymphocyte population throughout the body.

Memory T cells in humans are classically distinguished by the phenotype CD45RO+CD45RA−, and comprise heterogeneous populations of memory T cell subsets. [Farber, D L, et al. Nat. Rev. Immunol. (2014) 14 (1): 24-35] Naïve T cells uniformly express CCR7, reflecting their predominant residence in lymphoid tissue. Memory T cells are subdivided into CD45RA-CCR7+ central memory T (TCM) cells, which traffic to lymphoid tissues, and CD45RA-CCR7− effector memory T (TEM) cells, which can migrate to multiple peripheral tissue sites. Functionally, both Tcm and TEM cell subsets produce effector cytokines in response to viruses, antigens and other stimuli [Id., citing Wang A, et al. Sci Transl Med. (2012) 4: 149ra12030-33; Pedron B, et al. Pediatr Res. (2011) 69:106-111; Champagne P, et al. Nature. (2001) 410:106-111; Ellefsen K, et al. Eur J Immunol. (2002) 32:3756-3764], although TCM cells exhibit a higher proliferative capacity. (Id. citing Wang A, et al. Sci Transl Med. (2012) 4: 149ra120, Fearon D T, et al. Immunol Rev. 2006; 211:104-118). T memory stem (TSCM) cells, which resemble naïve T cells in that they are CD45RA+CD45RO− and express high levels of the co-stimulatory receptors CD27 and CD28, IL-7 receptor a chain (IL7Ra), CD62L and CCR7, have high proliferative capacity and are both self-renewing and multipotent in that they can further differentiate into other subsets, including Tcm and TEM cells [Id. citing Gattinoni L, et al. Nat Med. (2011) 17:1290-1297, Gattinoni L, et al. Clin Cancer Res. (2010) 16:4695-4701]. A progressive differentiation pathway based on signal strength and/or extent of activation, places naïve (Tx), T_{SCM}, T_{CM} and T_{EM} cells in a differentiation hierarchy, serving as precursors for effector T cells [Id. citing Gattinoni L, et al. Nat Rev Cancer. (2012) 12:671-684; Klebanoff C A, et al. Immunol Rev. (2006) 211:214-224; Lanzavecchia A, Sallusto F. Nat Rev Immunol. (2002) 2:982-987].

In mice, tissue resident memory T (TRM) cells are a non-circulating subset that resides in peripheral tissue sites and, in some cases, elicits rapid in situ protective responses. Mouse C D4+T_{R}M cells can be generated in the lungs from adoptive transfer of activated (effector) T cells [Id., citing Teijaro J R, et al. J Immunol. (2011) 187:5510-5514] or following respiratory virus infection [Id., citing Turner, D L, et al. Mucosal Immunol. (2014) 7 (3): 501-510], and are distinguished from splenic and circulating memory T cells by their upregulation of the early activation marker CD69, their tissue-specific retention in niches of the lung [Id., citing Turner, DL, ct al. Mucosal Immunol. (2014) 7 (3): 501-510] and their enhanced ability to mediate protection to influenza virus infection compared to circulating memory CD4+T cells [Id. citing Teijaro J R, et al. J Immunol. (2011) 187:5510-5514]. An analogous non-circulating CD4+T_{R}M cell subset has been identified in the bone marrow of mice following systemic virus infection that exhibits enhanced helper functions. [Id., citing Herndler-Brandstetter D, et al. J Immunol. (2011) 186:6965-6971]. CD8+T_{RM} cells generated following infection have been identified in multiple mouse tissues, including skin [Id., citing Clark R A, et al. Sci Transl Med. (2012) 4: 117ral17; Liu L, et al. Nat Med. (2010) 16:224-227], vaginal mucosa [Id., citing Mackay L K, et al. Proc Natl Acad Sci USA. (2012) 109:7037-7042, Shin H, Iwasaki A. Nature. (2012) 491:463-467], intestine [Id., citing Klonowski K D, et al. Immunity. (2004) 20:551-562, Masopust D, et al. J Exp Med. (2010) 207:553-564, Masopust D, et al. J Immunol. (2006) 176:2079-2083], lungs [Id., citing Turner, D L, et al. Mucosal Immunol. (2014) 7 (3): 501-510, Anderson K G, et al. J Immunol. (2012) 189:2702-2706] and brain [Id., citing Wakim L M, et al. Proc Natl Acad Sci USA. (2010) 107:17872-17879]. They are distinguished from splenic and circulating memory CD8+T cells by their increased expression of CD69 and by expression of the epithelial cell binding integrin aEB7 (also known as CD103 [Id, citing Mueller S N, et al. Annu Rev Immunol. (2013) 31:137-161, Mackay L K, et al. Proc Natl Acad Sci USA. (2012) 109:7037-7042, Casey K A, et al. J Immunol. (2012) 188:4866-4875; Masopust D, Picker L J. J Immunol. (2012) 188:5811-5817; Gebhardt T, Mackay L K. Front Immunol. (2012) 3:340].

In humans, memory CD4+T cells predominate throughout the body and persist as CCR7+ or CCR7-subsets localized to lymphoid tissues and mucosal sites, respectively, whereas memory CD8+T cells persist as mainly CCR7-subsets in all sites, with low numbers of CD8 Tcm cells in lymphoid tissues and negligible numbers of these cells in other sites [Id., citing Sathaliyawala T, et al. Immunity (2013) 38:187-197]. Most memory T cells in human mucosal, lymphoid and peripheral tissue sites such as skin express the putative T_{RM} cell marker CD69 [Id., citing Goronzy J J, Weyand C M. Nat Immunol. (2013) 14:428-436; Nikolich-Zugich J, Rudd B D. Curr Opin Immunol. (2010) 22:535-540; Clark R A, et al. J Immunol. (2006) 176:4431-4439, Mueller S N, et al. Annu Rev Immunol. (2013) 31:137-161, Casey K A, et al. J Immunol. (2012) 188:4866-4875], whereas circulating blood memory T cells uniformly lack CD69 expression. [Id., citing Sathaliyawala T, et al. Immunity (2013) 38:187-197].

Human T_{RM} cells also exhibit tissue-specific properties, suggesting in situ influences. For example, memory T cells in the small intestine and colon express the gut-homing receptor CCR9 [Id., citing Kunkel E J, et al. J Exp Med. (2000) 192:761-768] and the integrin α4β7 [Id., citing Agace W W. Trends Immunol. (2008) 29:514-522], and memory T cells in the lungs upregulate CCR6 expression [Id., citing Purwar R, et al. PLOS One. (2011) 6: e16245]. There is also evidence for crosstalk between mucosal sites, such as lung and intestines. For example lung dendritic cells induce migration of protective T cells to the gastrointestinal tract. [Id. citing Ruane D, et al. J Exp Med. (2013) 210:1871-1888].

There is evidence that T_{RM} can be multifunctional and also exhibit qualitative functional differences. A substantial fraction of human lung T_{RM} cells produce multiple pro-inflammatory cytokines [Id., citing Purwar R, et al. PLOS One. 2011; 6: e16245], and human intestinal T_{RM} cells are also multifunctional [Id. citing Sathaliyawala T, et al. Immunity. 2013; 38:187-197]. Other functions appear to be confined to specific subsets and/or tissue sites. For example, IL-17 is produced by a subset of CD4+T_{RM} cells in mucosal sites, particularly in intestines in healthy individuals [Id., citing Sathaliyawala T, et al. Immunity (2013) 38:187-, by CCR6+ memory T cells in peripheral blood [Id., citing Singh S P, et al. J Immunol. (2008) 180:214-221, Wan Q, et al. J Exp Med. (2011) 208:1875-1887], and by a subset of CD161+T cells in inflamed tissue, such as the skin of patients with psoriasis [Id., citing Cosmi L, et al. J Exp Med. 2008; 205:1903-1916]. Thus, while predominant memory T cell functions, such as IFNγ production, are broadly distributed among multiple memory T cell subsets and tissues, T_{RM} cells in tissue sites can adopt multiple or distinct functional attributes, which may also depend on tissue-specific inflammation.

Despite their specificity, human memory T cells exhibit cross-reactivity to antigenic epitopes not previously encountered, which may be due to intrinsic properties of TCR recognition [Id., citing Sewell A K. Nat Rev Immunol. 2012; 12:669-677] and to the range and breadth of human antigenic experience. For example, memory CD4+ and CD8+T cells specific for unique epitopes of avian influenza strain H5N1 were detected in healthy individuals that were not exposed to H5N1 infection assessed by serology [Id., citing Lee L Y, et al. J Clin Invest. (2008) 118 (10): 3478-90; Roti M, et al. J Immunol. (2008) 180:1758-1768]. In addition, HIV-specific memory T cells have been identified in HIV-negative individuals [Id., citing Su, L F et al. Immunity (2013) 38:373-83]. Virus-specific memory T cells also show cross-reactivity to alloantigens, autoantigens and unrelated pathogens [Id., citing D'Orsogna L J, et al. Transpl Immunol. (2010) 23:149-155, Wucherpfennig K W. Mol Immunol. (2004) 40:1009-1017]: EBV-specific human memory T cells generated in HLA-B8 individuals exhibit allogeneic cross-reactivity to HLA-B44 [Id., citing Burrows S R, et al. J Exp Med. (1994) 179:1155-1161], and influenza virus- and HIV-specific memory CD4+T cells recognize epitopes from unrelated microbial pathogens [Id., citing Su L F, et al. Immunity. (2013) 38:373-383]. Furthermore, T cells specific for the autoantigen myelin basic protein (MBP) recognized multiple epitopes from viral and bacterial pathogens [Id., citing Wucherpfennig K W. Mol Immunol. (2004) 40:1009-1017, Wucherpfennig K W, Strominger J L. Cell. (1995) 80:695-705]. This cross-reactivity may enable memory T cells to mediate protection without initial disease—a phenomenon known as “heterologous immunity” [Id., citing Welsh R M, Selin L K. Nat Rev Immunol. (2002) 2:417-Heterologous immunity has been demonstrated in humans where EBV infection expanded clones of influenza virus-specific T cells [Id., citing Clute S C, et al. J Clin Invest. 2005; 115:3602-3612].

Analysis of human samples has revealed that influenza-specific T_{RM} can be found in substantial numbers in lung tissue, highlighting their role in natural infection. Despite expressing low levels of granzyme B and CD107a, these CD8+T_{RM} had a diverse T cell receptor (TCR) repertoire, high proliferative capacities, and were polyfunctional [Muruganandah, V., et al. (2018). Front. Immunol., 9, 1574. doi: 10.3389/fimmu.2018.01574]. Influenza infection history suggests a greater level of protection against re-infections is likely due to the accumulation of CD8+T_{RM} in the lungs. Furthermore, the natural immune response to influenza A virus infection in a rhesus monkey model demonstrated that a large portion of influenza-specific CD8+T cells generated in the lungs were phenotypically confirmed as CD69+CD103+T_{RM}. Unlike lung parenchymal T_{RM}, airway CD8+T_{RM} are poorly cytolytic and participate in early viral replication control by producing a rapid and robust IFN-γ response. Bystander C D8+T_{RM} may also take part in the early immune response to infection through antigen non-specific, NKG2D-mediated immunity. The generation of functional T_{RM} that protect against heterosubtypic influenza infection appears to be dependent on signals from CD4+T cells. [Muruganandah, V., et al. (2018). Frontiers in Immunology, 9, 1574. doi: 10.3389/fimmu.2018.01574].

According to the paradigm of a typical CD8+ T cell response to acute viruses, CD8+T cells are effectors when an antigen is present and become memory when the antigen is eliminated. However, it has become apparent that in viral infections, a memory T cell population comprises multiple subtypes of cells, distributed in diverse anatomic compartments and possibly recirculating among them. The memory CD8+ T cell response to most viruses is diverse in phenotype and function and undergoes dynamic changes during its development and maintenance in vivo. This heterogeneity is related to the nature of the infecting virus, its cellular tropism, the anatomic location of the infection, and the location of the CD8+ T cells. In resolved acute infections, the presence of memory CD8+T cells at the site of the original virus entry and replication is crucial for a rapid response to a secondary infection. In latent infections, the presence of memory CD8+T cells at sites of virus persistence is important for immune surveillance of virus reactivation. [Racanelli, V. et al., Rev. Med. Virol. (2011) 21 (6): 347-357].

### II. Tumor Immunology

The term “tumor” includes cancer cells and the stroma supporting the cancer cells. Together they are often referred to as a “neoplasm”, meaning an abnormal mass of cells that persists and proliferates after withdrawal of the stimulus that initiated its appearance. Leukemias are cancers caused by neoplastic proliferations of blood cells, but usually do not form tumor masses. There are two types of neoplasms: benign and malignant. The common term for all malignant neoplasms is cancer. There is substantial evidence that malignant neoplasms/cancers are the results of multiple sequential mutations. As a result, certain molecules in cancer cells are mutant, up- or down-regulated, or no longer expressed. Most if not all cancers show epigenetic changes in gene expression. A cancer may require as many as 10 or more mutations to develop full malignancy.

The first stage of cancer development is called tumor initiation, which is generally assumed to be irreversible due to somatic mutations or germline mutations in various oncogene, tumor suppressor gene or DNA repair pathways. Initiated cells do not form tumors. However, initiated cells clonally expand to premalignant lesions evolving over many years.

This second protracted stage is driven by tumor promotion (i.e., exposure to promoting conditions or chemicals). The most advanced stage of these premalignant lesions is referred to as intraepithelial neoplasia or carcinoma in situ. The premalignant process ends with invasion, the appearance of the first cancer cells. Invasion usually precedes metastases and suffices as diagnostic criterion of cancer, although this criterion cannot be used for leukemia and mesenchymal tumors. There is molecular evidence for premalignant cells spreading to distant sites where these cells remain premalignant unless promoted to become malignant. Invasion may or may not be followed by cancer cells entering the lymphatics, bloodstream or fluid of the coelomic cavities to implant at sites discontinuous with the original tumor. Metastasis defines a tumor as malignant; benign tumors do not metastasize. [Paul, William E. Fundamental Immunology 7^{th} Ed. Wolters Kluwer Health/Lippincott Williams & Wilkins, Philadelphia (2013), Chapter 47, pp. 1200-1201; 1220-1222].

The term “tumor progression” describes the third phase of the multistep process. Invasive growth of a lesion usually ends with a highly aggressive, widely metastatic cancer that ultimately kills the host. There is compelling evidence that most cancers are clonal in origin and that in cancer progression, new subpopulations of cells arise continuously due to Darwinian selection of genetic variants that have a growth advantage, escape homeostatic controls, or resist destruction by defense mechanism or treatment. During this evolution, sequential mutations result in changes in rate of growth, morphology, hormone dependence, enzyme and cytokine production and expression of surface antigens. [Paul, William E. Fundamental Immunology 7th Ed. Wolters Kluwer Health/Lippincott Williams & Wilkins, Philadelphia (2013), Chapter 47, pp. 1200-1201; 1220-1222].

### Factors Limiting Cancer Immunity

Various mechanisms allow cancers to escape innate or adaptive host immunity by (1) inducing a protective and supportive stromal microenvironment; (2) increasing resistance to direct immune attack; and (3) inducing T cell anergy. Often, cancer cells seem to use a combination of the three.

Tumor microenvironment. Many escape variants keep their antigens but induce a stroma more effective than the parental cells in supporting growth and protecting against destruction by the host.

Regulatory T cells (Tregs) play a role in tolerance to tumor antigens as well as the resistance of tumors to immune-mediated elimination [Mendelsohn, J., Gray, JW, Howley, P M, Israel, MA, and Thompson, CB. The Molecular Basis of Cancer, Chapter 52, pp., 715-17, citing Curiel, T J et al. Nat. Med. (2004) 10:942-9; Liyanage, U K et al. J. Immunol. (2002) 169:2756-61]. Tregs are characterized by expression of a central master regulatory transcription factor (FoxP3); although CD4+Tregs selectively express a number of cell-membrane molecules, including CD25, neuropilin (a surface marker of Tregs, GITR and LAG-3 [Id., citing Bruder, D. et al. Eur. J. Immunol. (2004) 34:623-3078-80], their overall inhibitory capacity is absolutely dependent on sustained expression of FOXp3 [Id., citing Williams L M et al. Nat. Immunol. (2007) 8:277-84; Zheng, Y. and Rudensky, AY. Nat. Immunol. (2007) 8:457-62]. Mechanisms of immune suppression by Tregs vary and include production of inhibitory cytokines, such as IL-10 and IL35. [Id., citing Vlovw, G C et al. N. Engl. J. Med. (2000) 342:1350-8].

The tumor microenvironment contains multiple inhibitory cells and molecules. Immature myeloid cells (iMCs) [Mendelsohn, J., Gray, JW, Howley, P M, Israel, MA, and Thompson, CB. The Molecular Basis of Cancer, Chapter 52, pp., 715-17, citing Kusmartsec, S. and Gabrilovich, DI. Cancer Immunol. Immunother. (2006) 55:237-45; Yong, M R et al. Hum. Immunol. (2001) 62:332-41, often termed myeloid-derived suppressor cells (MDSCs) (Id., citing Bronte, V. et al. Blood (2000) 96:3838-46; Bronte, V. et al. J. Immunol. (2003) 170:270-8; Mazzoni, A. et al. J. Immunol. (2002) 168:689-95; Zea, A H et al. Cancer Res. (2005) 65:3044-8) represent myeloid cell types, somewhat overlapping with tumor-associated macrophages (TAMs) [see Zhang, Z. et al. Front. Cell & Devel. Biol. (2020) 8:17; Bai, R. et al. Front. Oncology (2020) 10:1290], which share the common feature of inhibiting both the priming and effector function of tumor-reactive T cells. It is not clear whether these myeloid cell types represent distinct lineages or different states of the same general immune inhibitory cell subset. [Pardoll, D. “Cancer Immunotherapy with vaccines and checkpoint blockade.” In The Molecular Basis of Cancer, Chapter 52, Mendelsohn, J., Gray, JW, Howley, P M, Israel, MA, and Thompson, CB. pp., 715-17]. In mice, iMCs and MDSCs are characterized by coexpression of CD11b (considered a macrophage marker) and Grl (considered a granulocyte marker) while expressing low or no MHC class II or costimulatory molecule CD86. In humans, they are defined as CD33+ by lack of markers of mature macrophages, DCs or granulocytes and are DR-. A number of molecular species produced by tumors tend to drive iMC/MSC accumulation. These include IL-6, macrophage colony stimulating factor (CSF-1), IL-10 and ganglosides. IL-6 and IL-10 are potent inducers of STAT3 signaling, which has been shown to be important in iMC/MDSC persistence and activity.

In addition to inhibitory cytokine production, myeloid cells of multiple type in the tumor microenvironment express a number of enzymes whose metabolic activity ultimately results in inhibition of T cell responses within the tumor microenvironment. These include the production of reactive oxygen species (ROS) and/or reactive nitrogen species (RNS). Nitric oxide (NO) production by iMC/MDSC as a result of arginase and iNOS activity has been well-documented, and inhibition of this pathway with a number of drugs can mitigate the inhibitory effects of iMC/MDSC. ROS, including H₂O₂, have been reported to block T-cell function associated with the down-modulation of the & chain of the TCR signaling complex [Id. citing Schmiclau, J. and Finn, OJ. Cancer Res. (2001) 61:4756-60], a phenomenon associated with generalized T-cell unresponsiveness.

Another mediator of T-cell unresponsiveness associated with cancer is the production of indoleamine 2,3-dioxygenase (IDO) [Id., citing Munn, D H et al. Science (2002) 297:18;67-70]. IDO appears to be produced by DCs either within tumors or in tumor-draining lymph nodes. IDO in DCs has been reported to be induced via backward signaling by B7-1/2, homologous costimulatory ligands expressed on the surface of antigen presenting cells (APCs), which bind to the CD28 family of receptors on lymphocytes and regulate immune responses via costimulatory or coinhibitory signals [Collins, M. et al. Genome Biol. 6 (6): 223], on ligation with CTLA-4. [Id, citing Baban, B. et al. Int. Immunol. (2005) 1; 7:909-19; Mellor, A L et al. Int. Immunol. (2004) 16:1391-1401]. While the major IDO-producing DC subset is either a plasmacytoid DC (PDC) or a pDC related cell that is B220+, an isoform of CD45 [Id., citing Munn, D H et al. J. Clin. Invest. (2004) 114:280-90], IDO has been shown to be expressed by multiple cell types in the immune microenvironment, including tumor cells themselves [Id., citing Uyttenhove, C. et al. Nat. Med. (2003) 9:1269-74]. IDO appears to inhibit T-cell responses through catabolismof tryptophan; activated T cells are highly dependent on tryptophan and are therefore sensitive to tryptophan depletion. A bystander mechanism has been proposed, whereby DCs in the local environment deplete tryptophan via IDO upregulation, thereby inducing metabolic apoptosis in locally activated T cells. [Id., citing Munn, D H et al. Science (2002) 297:1867-70]. A second tryptophan-metabollizing enzyme is tryptophan dioxygenase (TDO), which is upregulated commonly in human cancers, and which may inhibit antitumor responses within the microenvironment similar to IDO. [Id., citing Opitz, C A et al. Nature (2011) 478:197-203]. Further, a major product of IDO and TDO metabolism of tryptophan—kynurenine—has potent effects on T cell differentiation. Under some circumstances, kynurenine can promote Treg development [Id., citing Mezrich, J D et al. J. Immunol. (2010) 185:3190-8], and under other circumstances, it can promote development of TH17 cells [Id., citing Favre, D. et al. Sci. Trans. Med. (2010) 2: 32ra36], known for production of IL-17 and for its procarcinogenic properties.

A major inhibitory cytokine produced by iMC/MDSCs and by other cell types implicated in blunting antitumor immune responses is transforming growth factor beta (TGF-β), which has pleiotropic physiological effects. For most normal epithelial cells, TGF-β is a potent inhibitor of cell proliferation, causing cell cycle arrest in the G1 stage [Id., citing Blobe, G C et al. N. Engl. J. Med. (2000) 342:1350-8]. In many cancer cells, mutations in the TGFβ pathway confer resistance to cell cycle inhibition, allowing uncontrolled proliferation.

### Increasing Resistance to Direct Immune Attack

Primary or intrinsic resistance represents a clinical situation in which a malignant tumor does not respond to immune therapy. Hyperprogression (HPD) is a form of primary resistance. For example, blockade of immune checkpoints has the potential to functionally stimulate Tregs, locally creating an immunosuppressive microenvironment; has the potential to lead to polarization of immunosuppressive cells, producing immunosuppressive cytokines, and has the potential to stimulate THI and TH17-mediated inflammatory responses, thus creating conditions for accelerated tumor growth and resistance to immunotherapy. [Bai, R. et al. Front. Oncology (2020) 10:1290, citing Champiat, S. et al. Clin. Cancer Res. (2017) 23:1920-8].

Acquired resistance represents a clinical situation in which a tumor can initially respond effectively to immunotherapy but relapses or progresses after a period of treatment. Adaptive immune resistance is a mechanism by which a tumor can be recognized by the immune system, but can evade immunity by altering itself to adapt to immune attack. [Id., citing Sharma, P.et al. Cell (2017) 168:707-23].

Intrinsic mechanisms of tumor immune resistance include changes in antitumor immune response pathways, alteration of signaling pathways in tumor cells, and other changes in tumor cells that lead to an inhibitory immunosuppressive microenvironment. For example, tumor cells often inhibit T cell activation by reducing or losing antigen expression [Id., citing de Vries, T J et al. Cancer Res. (1997) 57:3223-9] and regulate autoantigenicity by means of endocytic antigens or antigen shedding to mediate immune escape. In addition, the host can selectively eliminate tumor-specific antigen (TSA)-expressing cells and to some extent promote the generation of tumor antigen-losing variants [Id., citing DuPage, M. et al. Nature (2012) 482:405-9]. Tumor cells also can undergo antigenic drift leading to epitope mutations that alter the antigenicity of tumor cells, which subsequently escape T cell-mediated attack [Id., citing Bai, X-F et al. J. Clin. Invest. (2003) 111:487-96].

Disruption of the antigen presentation signal pathway can render T cells unable to activate and lead to immune escape, including mutations that interfere with the proteasome, a multienzyme complex involved in the ubiquitin-proteasome pathway control of cell cycle progression, in the termination of signal transduction cascades, and in the removal of mutant, damaged, and misfolded proteins, transporters involved in antigen processing, and changes in the structural composition of MHC itself. The abnormalities of MHC molecules are broadly divided into structural defects due to gene mutations, such as mutations within the receptor-binding domain of MHC cells [Id., citing Giannakis, M. et al. Cell Rep. (2016) 15:857-65], and defects in regulatory mechanisms due to epigenetic changes, e.g., those that are associated with the downregulation of transporters associated with antigen processing, the downregulation of low molecular mass proteins and methomyl proteins and the inactivation of class I MHC gene transcription [Id., citing Sokol, L. et al. J. Transl. Med. (2015) 13:279]. For example, in certain malignancies, such as advanced multiple myeloma, tumor cells can escape the lysis mediated by cytotoxic T lymphocytes (CTLs) and NK cells by overexpressing non-classical MHC-I molecules (e.g., HLA-G), leading to the development of immune escape [Id., citing Gao, M. et al. Acta Biochim. Biophys. Sinica (2014) 46:597-604].

In patients receiving immunotherapy, tumor cells can downregulate or alter IFN-gamma signaling pathways such as loss-of-function alleles of genes encoding for JAK1/2 and changes in STATI to escape the influence of IFN-gamma, resulting in resistance, and epigenetic alterations associated with the IFN-gamma signaling pathways can affect immune resistance. For example, activating mutations in tyrosine-protein phosphatase non-receptor type 2 (Ptpn2), which negatively regulates JAKI and STATI signaling has also been associated with primary resistance to PD-1 blockade via resistance to IFN-gamma [Id., citing Manguso, R T et al. Nature (2017) 547:413-18].

### Inducing T Cell Anergy

T cell anergy is a tolerance mechanism in which the lymphocyte is intrinsically functionally inactivated following an antigen encounter, but remains alive for an extended period of time in a hyporesponsive state. Models of T cell anergy affecting both CD4 (+) and CD8 (+) cells fall into two broad categories. One, clonal anergy, is principally a growth arrest state, whereas the other, adaptive tolerance or in vivo anergy, represents a more generalized inhibition of proliferation and effector functions.

Clonal anergy arises from incomplete T cell activation, is mostly observed in previously activated T cells, is maintained by a block in the Ras/MAP kinase pathway, can be reversed by IL-2 or anti-OX40 signaling, and usually does not result in the inhibition of effector functions. [Schwartz, RH. Annu. Rev. Immunol. (2003) 21:305-34].

In vivo anergy is most often initiated in naïve T cells in vivo by stimulation in an environment deficient in costimulation or high in coinhibition. The cells proliferate and differentiate to varying degrees and then downregulate both functions in the face of persistent antigen. The state involves an early block in tyrosine kinase activation, which predominantly inhibits calcium mobilization, and an independent mechanism that blocks signaling through the IL-2 receptor. Adaptive tolerance reverses in the absence of antigen. [Schwartz, RH. Annu. Rev. Immunol. (2003) 21:305-34].

For example, anergy is a component of normal B-cell behavior. [Packham, G. et al. Haematologica (2014) 99 (7): 1138-48, citing Cambier, J C et al. Nat. Rev. Immunol. (2007) 7 (8): 633-43]. It is a state of cellular lethargy resulting from binding of antigen by B cells (signal 1) in the absence of significant CD4+T-cell help (signal 2). In effect, the B cells are left suspended in an unresponsive state and are prone to apoptosis.

However, in chronic lymphocytic leukemia (CLL}, the predominant B cell receptor response in vivo appears to be anergy, whereby autoreactive B cells are rendered nonresponsive to activation via their cell surface BCRs. Unlike normal B cells, CLL cells and their progeny are protected from death, with expression of the anti-IgM-inducible survival-promoting MCL1 protein detected in CLL blood cells and, like MYC, correlating with progressive disease. [Id., citing Petlickovski, A. et al. Blood (2005) 105 (12): 4820-7; Pepper, C. et al. Blood (2008) 112 (9): 3807-17]. In CLL, two major subsets arise at distinct points of differentiation and express unmutated or mutated IGHV genes: U-CLL and M-CLL, respectively. The clinical behavior of the two subsets differs substantially, with U-CLL having a poorer prognosis. [Id., citing Hamblin, T J et al. Blood (1999) 94 (6): 1848-54; Damle, R N et al. Blood (1999) 94 (6): 1840-7]. Extended survival also appears to occur in the anergic fraction, mainly M-CLL; this runs counter to the known vulnerability of normal anergic B cells which are short-lived in vivo. [Id., citing Cambier, J C et al. Nat. Rev. Immunol. (2007) 7 (8): 633-43]. Although anergy is observed in CLL cells, especially M-CLL, there is a proliferative fraction in all CLL patients. [Messmer, BT ct al. J. Clin. Invest. (2005) 115 (3): 755-64]. This positive response to antigen engagement occurs in a minority of cells located in specific tissue sites, particularly within the proliferation center of lymph nodes (LN) [Id., citing. Soma, L A et al. Hum. Pathol. (2006) 37 (2): 152-9]

### III. Hematopoiesis, Clonal Hematopoiesis and Acute Myeloid Leukemia

The term “hematopoiesis” as used herein refers to the formation of new blood cells. The classical unidirectional model of hematopoietic differentiation involves differentiation into increasingly lineage-specific progenitors, although there may also be alternate pathways that are used separately or in combination with classical pathways. In the classical pathway, long-term repopulating hematopoietic stem cells (LTR-HSCs) are characterized by their ability to self-renew and differentiate into cells that are multipotent. Multipotent progenitors (MPPs) have reduced self-renewal capacity and differentiate into common lymphoid progenitors (CLPs) or common myeloid progenitors (CMPs). The CMP differentiates into all the blood lineages except for lymphoid. The commitment of hematopoietic cells to increasingly lineage-restricted cells requires cytokine stimulation and regulation by transcription factors. This model has some shortcomings in that it oversimplifies the complexity of hematopoictic stem and progenitor cells (HSPCs), and it is only based on the surface markers and transplantation using bulk cells. Bulk cell analysis assumes that each cell, which has the same phenotype, possesses an identical function.

This classical model has been challenged over the past several years, especially in the elucidation of megakaryopoiesis, Advances in single cell technology and genetic mouse models have broadened our knowledge of hematopoiesis. and new types of HSPCs have been identified and extensively studied due to their lineage biases. FIG. 2 schematically depicts a reconciled model for hematopoietic stem cell differentiation HSCs first differentiate into MPP1/short-term HSCs (ST-HSC), then give rise to MPP2, MPP3 and MPP4 lymphoid primed MPPs (LMPP). MPP2 can generate premegakaryocye/erythroid progenitor (pre MegE), and subsequently, pre MegE gives rise to platelets through megakaryocyte progenitors (MkPs) or produce erythrocytes through Pre CFU-E. MPP3 mostly give rise to granulocyte and monocyte lineages, and MPP4 (LMPP) mainly contribute to lymphocytes. [Cheng, H. et al. New paradigms on hematopoietic stem cell differentiation. Protein Cell (2020) 11 (1): 34-44].

### Clonal Hematopoiesis in Healthy Individuals

With every round of mitotic division, DNA damage and inefficient repair occur, resulting in the accumulation of somatic mutations in aging hematopoietic stem and progenitor cells (HSPCs) over time. The process of acquired mutations leading to clonal expansion of HSPCs is referred to as clonal hematopoiesis (CH). CH is a risk factor for the development of hematologic malignancies, most commonly myeloid neoplasms (MNs) including acute myeloid leukemia (AML), myelodysplastic syndrome (MDS), and myeloproliferative neoplasm (MPN). The mutations driving CH confer a survival advantage over wild-type cells that results in the clonal expansion of the mutated cell. CH driven by single nucleotide variants (SNVs) or insertion-deletion mutants (indels) are most commonly drivers of MNs. This includes epigenetic modifiers (DNMT3A, TET2, ASXL1, IDH1, IDH2), splicing factors (SF3B1, SRSF2, U2AF1), and genes involved in the DNA damage response (TP53, PPMID, CHEK2), and JAK2). Copy number events (amplifications, deletions, or copy neutral loss of heterozygosity) are also known to drive CH. Other classes of mutational events such as medium-sized (>50 bp) copy number events, structural events and noncoding mutations have relevance in hematologic cancer and may also drive CH, yet are not well characterized. [Chan, I C C, et al. Front. Oncol. (2022) 12:2022.

### Acute Myeloid Leukemia (AML)

Acute myeloid leukemia (AML) is a malignant disease of the bone marrow in which hematopoietic precursors are arrested in an early stage of development. Most AML subtypes are distinguished from other related blood disorders by the presence of more than 20% blasts in the bone marrow.

The underlying pathophysiology in AML consists of a maturational arrest of bone marrow cells in the earliest stages of development. The mechanism of this arrest is under study, but in many cases, it involves the activation or inactivation of genes through chromosomal translocations and other genetic and/or epigenetic abnormalities. [Seiter, K., medscape.com/article/197802, updated Dec. 6, 2022, citing Arber, D A et al. Blood (2016) 127 (20): 2391-405; Smith M T et al. IARC Sci. Publ. (2004) 373-92; Ghiaur, G. et al. Semin. Hematol. (2015) 52 (3): 200-6]. This developmental arrest results in two disease processes. First, the production of normal blood cells markedly decreases, which results in varying degrees of anemia, thrombocytopenia, and neutropenia. Second, the rapid proliferation of the abnormal myeloblasts, along with a reduction in their ability to undergo programmed cell death (apoptosis), results in their accumulation in the bone marrow, the blood, and, frequently, the spleen and liver.

### Etiology.

Several factors have been implicated in the causation of AML, including antecedent hematologic disorders, familial syndromes, environmental exposures, and drug exposures. However, most patients who present with de novo AML have no identifiable risk factor.

Antecedent hematologic disorders. The most common risk factor for AML is the presence of an antecedent hematologic disorder, the most common of which is myelodysplastic syndrome (MDS). Other antecedent hematologic disorders that predispose patients to AML include aplastic anemia and myeloproliferative disorders, especially myelofibrosis.

Congenital disorders. Some congenital disorders that predispose patients to AML include Bloom syndrome, Down syndrome, congenital neutropenia, Fanconi anemia, and neurofibromatosis. Usually, these patients develop AML during childhood; rarely, they may present in young adulthood.

More subtle genetic disorders, including polymorphisms of enzymes that metabolize carcinogens, also predispose patients to AML. For example, polymorphisms of NAD (P) H: quinone oxidoreductase (NQO1), an enzyme that metabolizes benzene derivatives, are associated with an increased risk of AML. [Id., citing Larson, R A et al. Blood (1999) 94 (2): 803-7] Particularly increased risk exists for AML that occurs after chemotherapy for another disease or for de novo AML with an abnormality of chromosomes 5, 7, or both. Likewise, polymorphisms in glutathione S-transferase are associated with secondary AML after chemotherapy for other malignancies. [Id., citing Allan, J M et al. Proc Natl Acad. Sci. USA (2001) 98 (20): 11592-7].

Familial syndromes. With the routine use of expanded next-generation genetic panels on bone marrow, and with confirmation on nonhematopoietic tissues, many more patients are being diagnosed with germline mutations that predisposed them to AML. Among these genes are DDX41 (which regulates the expression and alternative splicing of genes involved in tumorigenesis and immune responses), SRP72 (a component of the signal recognition particle responsible for arresting translation of nascent proteins destined for the cell membrane or extracellular secretion and transferring them to the endoplasmic reticulum (ER) for correct trafficking); ANKRD26, which encodes a protein containing N-terminal ankyrin repeats which function in protein-protein interactions, and ETV6, which provides instructions for producing a protein that functions as a transcription factor. [Id., citing Guidugli, L. et al. Leukemia (2017) 31 (5): 1226-9]

Environmental exposures. Several studies demonstrate a relationship between radiation exposure and leukemia. In several studies, the risk of AML was slightly increased in people who smoked compared with those who did not smoke. Persons who smoke tobacco have a small but statistically significant (odds ratio, 1.5) increased risk of developing AML. [Id., citing Brownson, R C et al. Am. J. Epidemiol. (1991) 134 (9): 938-41]. Exposure to benzene is associated with aplastic anemia and pancytopenia; these patients often develop AML. Many of these patients have the erythroleukemia subtype of AML (AML-M6). Exposure to soot, creosote, inks, dyes, and tanning solutions and coal dust have also been associated with AML. [Id., citing Poynter, J N et al. Intl J. Cancer (2016) 140 (1): 23-33.]

Previous exposure to chemotherapeutic agents. As more patients with cancer survive their primary malignancy and more patients receive intensive chemotherapy (including bone marrow transplantation [BMT], the number of patients with AML increases because of exposure to chemotherapeutic agents.

### Prognosis

The prognosis of AML depends on several factors. Increasing age is an adverse factor, because older patients more frequently have a previous antecedent hematologic disorder and/or poor-risk cytogenetic and molecular markers that make the leukemia resistant to chemotherapy. Older patients also frequently have comorbid medical conditions that compromise the ability to tolerate full doses of chemotherapy. A previous antecedent hematologic disorder (most commonly, MDS) is associated with a poor outcome of therapy.

Findings from cytogenetic analysis of the bone marrow constitute one of the most important prognostic factors. Patients with t (8;21), t (15;17), or inversion 16 have the best prognosis, with long-term survival rates of approximately 65%. Patients with normal cytogenetic findings have an intermediate prognosis and have a long-term survival rate of approximately 35%. Patients with poor-risk cytogenetic findings (especially −7, −5, or monosomal karyotype) have a poor prognosis, with a long-term survival rate of less than 10%. [Id., citing Valk, P J et al. N. Engl. J. Med. (2004) 350 (16): 117-28]

Other cytogenetic abnormalities, including +8, 11q23, and miscellaneous, have been reported to confer intermediate risk in some series and poor risk in others.

The presence of an FLT3 mutation is associated with a poorer prognosis. There are two major types of FLT3 mutations: internal tandem duplication mutations in the juxtamembrane domain (FLT3-ITD) and point mutations or deletion in the tyrosine kinase domain (FLT3-TKD). [Kiyoi, H. et al. Cancer Sci. (2020) 111 (2): 312-322]. Both mutant FLT3 molecules are activated through ligand-independent dimerization and trans-phosphorylation.

Biallelic mutations in a gene that provides instructions for making a protein called CCAAT enhancer-binding protein alpha (CEBPA), a transcription factor involved in the maturation of certain blood cells, are associated with a longer remission duration and longer overall survival. [Seiter, K., medscape.com/article/197802, updated Dec. 6, 2022, citing Taskesen, E. et al. Blood (2011) 117 (8): 2469-75].

Mutations in a gene that encodes nucleophosmin (NPM) are associated with an increased response to chemotherapy. Nucleophosmin, also known as B23, No38 or numatrin, is an abundant nucleolar protein found in the nuclei of proliferating cells.

Patients with TP53 mutations have a particularly poor prognosis. [Id., citing Taylor, J. et al. Blood (2017) 130 (4): 410-23]

Using the European LeukemiaNet (ELN) molecular-risk classification of patients with primary cytogenetically normal AML, a study by Metzeler et al. determined that mutations to the TET2 gene, which provides instructions for making a protein of unknown function but that is believed to be involved in regulating transcription, had an adverse prognostic impact in an otherwise favorable-risk patient subset,. [Id., citing Metzeler, K H et al. J. Clin. Oncol. (2011) 29 (1): 1373-81]

In adults, treatment results are generally analyzed separately for younger (18-60 years old) patients with AML and for older patients (>60 years old). With current standard chemotherapy regimens, approximately 40-45% of adults younger than 60 years survive longer than 5 years and are considered cured. Results in older patients are more disappointing, with fewer than 10% surviving over the long term. Overall, cure rates for younger patients have improved over the past few decades, but little progress has been made in improving the survival of older patients. [Id., citing Bower, H. et al. Blood Cancer J. (2016) 6: c390].

The prognosis of therapy-related AML is particularly poor, with 5-year survivals of approximately 10%. The prognosis is better for the subset of patients with therapy-related AML who have favorable cytogenetic abnormalities. [Id., citing McNerney, M E et al. Nat. Rev. Cancer (2017) 17 (9): 513-27; Kayser, S. et al. Blood (2011) 117 (7): 2137-45].

### Cytogenetic Analysis

Guidelines from an international expert panel, on behalf of the European LeukemiaNet (ELN), recommend risk stratification for patients with AML on the basis of genetic abnormalities. The ELN identifies three levels of risk: favorable, intermediate, and adverse. [Id., citing Dohner, H. et al. Blood (2017) 129 (4): 424-47]

Genetic Abnormalities that Convey Favorable Risk are as Follows:
t (8;21) (q22;q22.1); RUNX1-RUNXIT1
inv (16) (p13.1q22) ort (16;16) (p13.1;q22); CBFB-MYH11
Mutated NPM1 without FLT3-ITD or with low allelic ratio (<0.5) FLT3-ITD
Biallelic mutated CEBPA

Genetic abnormalities that convey intermediate risk are as follows:
Mutated NPM1 and high allelic ratio (≥0.5) FLT3-ITD
Wild-type NPM1 without FLT3-ITD or with low allelic ratio FLT3-ITD (without adverse-risk genetic lesions)
t (9;11) (p21.3;q23.3); MLLT3-KMT2A

Genetic abnormalities that convey adverse risk are as follows:
t (6;9) (p23;q34.1); DEK-NUP214
t (v;11q23.3); KMT2A rearranged
t (9;22) (q34.1;q11.2); BCR-ABL1
inv (3) (q21.3q26.2) or t (3;3) (q21.3;q26.2); GATA2, MECOM (EVI1)
−5 or del (5q); −7; −17/abn (17p)
Complex karyotype or monosomal karyotype
Wild-type NPM1 and high allelic ratio FLT3-ITD
Mutated RUNX1
Mutated ASXL1
Mutated TP53

Several molecular abnormalities that are not detected with routine cytogenetics have been shown to have prognostic importance in patients with AML.

Next-generation sequencing identified multiple recurrent somatic mutations in >90% of patients with AML. [Kantarjiian, H. et al. Blood Cancer J. (2021) 11:41, citing Papaemmanuil, E. et al. N. Engl. J. Med. (2016) 374:2209-21; Angenendt, L. et al. J. Clin. Oncol. (2019) J. Clin. Oncol. 3; 7:2632-42]. Frequently mutated genes (frequency >5%) are: FLT3, NPM1, DNMT3A, IDHI, IDH2, TET2, RUNX1, p53; NRAS; CEBPA, and WTI. [Id., citing Papaemmanuil, E. et al. N. Engl. J. Med. (2016) 374:2209-21; Richard-Carpentier, G. and DiNardo, CD. Hematol. Am. Soc. Hematol. Educ. Program (2019) 548-56 Angenendt, L. et al. J. Clin. Oncol. (2019) J. Clin. Oncol. 3; 7:2632-42]. Based on functional analysis and known pathways, these are grouped into biological-functional categories: myeloid transcription factor fusions or mutations; NPM1 mutations; tumor-suppressor gene mutations; epigenome-modifyiing gene mutations; activated signaling pathway gene mutations; cohesion-complex gene mutations; and spliceosome-complex gene mutations. [Kantarjiian, H. et al. Blood Cancer J. (2021) 11:41].

Translating to clinical practice, the important molecular subsets are based on the identification of an FLT3 mutation (30% of AML), NPMI mutation (40-50% of normal karyotype AML); isocitrate dehydrogenase 1 or 2 (IDH1/2) mutations (20% of AML) and TP53 mutations (2 to 20% of AML). [Id].

Patients with NPMI-mutated AML have a more favorable prognosis; those with FLT3-ITD mutations have a poor prognosis, especially among patients with high FLT3 allelic ratios (ARs) and in the absence of NPMI mutation. Patients with diploid karyotype AML (without adverse mutations such as TP53, or ASXL1) and biallelic CEBPA mutations (2% or less of AML) have a favorable prognosis. [Id., citing Dohner, H. et al. N. Engl. J. Med. (2015) 373:1136-52].

FLT3 is the most commonly mutated gene in cases of AML and is constitutively activated in one third of AML cases. [Id., citing Griswold, I J et al. Blood (2004) 104 (9): 2912-8] Internal tandem duplications (ITDs) in the juxtamembrane domain of FLT3 exist in 25% of AML cases. In other cases, mutations exist in the activation loop of FLT3. Most studies demonstrate that patients with AML and FLT3-ITDs have a poor prognosis.

Mutations in NPMI are associated with increased response to chemotherapy in patients with a normal karyotype. [Id., citing Falini, B. et al. N. Engl. J. Med. (2005) 352 (30:254-66]. In a study by Thiede et al. of FLT3 and NPM1 in 1485 patients with AML, analysis of the clinical impact in 4 groups (NPMI and FLT3-ITD single mutants, double mutants, and wild-type [wt] for both) revealed that patients having only an NPMI mutation (without a FLT3-ITD) had a significantly better overall survival and disease-free survival and a lower cumulative incidence of relapse. [Id., citing Thiede, C. et al. Blood (2002) 99 (12): 4326-35].

Mutations in CEBPA are detected in 15% of patients with normal cytogenetics findings. Biallelic mutations are associated with a longer remission duration and longer overall survival. [Id., citing Frohling, S. et al. J. Clin. Oncol. (2004) 22 (4): 624-33]. ERG overexpression is an adverse predictor in cytogenetically normal AML.

A study by the Cancer and Leukemia Group B (CALGB) found that high brain and acute leukemia cytoplasmic protein [BAALC] expression was associated with FLT3-ITD, wild-type NPM1, mutated CEBPA, mixed-lineage leukemia-partial tandem duplication gene (MLL-PTD), absent an FLT3 mutation in the tyrosine kinase domain (FLT3-TKD) and high ERG expression. In a multivariate analysis, high BAALC expression independently predicted lower complete remission rates when ERG expression and age were adjusted for and shorter survival when FLT3-ITD, NPM1, CEBPA and WBC count were adjusted for. [Id., citing Schwind, S. ct al. Blood (2010) 116 (25): 5660-9].

### Staging Acute Myeloid Leukemia:

The term “staging” generally describes or classifies a cancer based on how much cancer there is in the body and where it is when first diagnosed. This is often called the extent of cancer.

Two staging systems are commonly used for AML. The French-American-British (FAB) classification system is based on morphology to define specific immunotypes. The World Health Organization (WHO) classification reviews chromosome translocations and evidence of dysplasia. [Id., citing Arber, D A et al. Blood (2016) 127 (20): 2391-405].

Because acute myeloid leukemia (AML) starts in the bone marrow and is usually not detected until it has spread to other organs, traditional cancer staging is not needed. Rather than using the common T (size of the tumor and any spread of cancer into nearby tissue); N (spread of cancer to nearby lymph nodes) M (metastasis), method for evaluating the cancer, the subtype of AML is classified using a cytologic (cellular) system. The traditional French-American-British (FAB) classification of AML is as follows:

M0-Undifferentiated leukemia, In this stage of acute myelogenous leukemia, the bone marrow cells show no significant signs of differentiation

M1-Myeloblastic without differentiation, Bone marrow cells show some signs of granulocytic differentiation with or without minimal cell maturation.

M2-Myeloblastic with differentiation-maturation of the bone marrow cells is beyond the promyelocyte (early granulocyte) stage. Varying amounts of granulocyte maturation may be observed.

M3-Promyelocytic, Most of the abnormal cells are early granulocytes, between myeloblasts and myelocytes in their stage of development. The cells contain many small particles and have nucleuses of varying size and shape.

M4-Myelomonocytic; M4co-Myelomonocytic with cosinophilia: In this stage of AML, the bone marrow and circulating blood have variable amounts of monocytes and differentiated granulocytes in them. The percentage of monocytes and promonocytes in the bone marrow is greater than 20 percent. There may also be an increased number of cosinophils, a type of granulocyte that often has a two-lobed nucleus.

M5-Monoblastic leukemia; M5a-Monoblastic without differentiation; M5b-Monocytic with differentiation: This subset is further divided into two different categories. The first is characterized by poorly differentiated monoblasts with lacy-appearing genetic material. The second subset is characterized by a large number of monoblasts, promonocytes and monocytes. The proportion of monocytes in the bloodstream may be higher than that in the bone marrow.

M6-Erythroleukemia: This form of leukemia is characterized by abnormal red blood cell-forming cells, which make up over half of the nucleated cells in the bone marrow.

M7-Megakaryoblastic leukemia: The blast cells in this form of leukemia look like immature megakaryocytes (giant cells of the bone marrow) or lymphoblasts (lymphocyte-forming cells). M7 leukemia may be distinguished by extensive fibrous tissue deposits (fibrosis) in the bone marrow.

The newer WHO classification is as follows [Id., citing Arber, D A et al. Blood (2016) 127 (20): 2391-405]:
AML with recurrent genetic abnormalities: AML with t (8;21) (q22;q22), (AMLI/ETO); AML with abnormal bone marrow cosinophils and inv (16) (p13q22) or t (16;16) (p13) (q22), (CBFB/MYH11); APL with PML/RARa; AML with t (9;11) (p21.3;q23.3), (MLLT3-KMT2A); AML with t (6;9) (p23;q34.1), (DEK-NUP214); AML with inv (3) (q21.3q26.2) or t (3;3) (q21.3;q26.2), (GATA2, MECOM); AML (megakaryoblastic) with t (1;22) (p13.3q133), (RBM15-MKLI), AML with BCR-ABLI; AML with mutated NPM1; AML with biallelic mutations of CEBPA. AML with mutated RUNX1;
AML with myelodysplasia-related changes (following myelodysplastic syndrome (MDS) or MDS/myeloproliferative disease (MPD); without antecedent MDS or MDS/MPD but with dysplasia in at least 50% of cells in 2 or more lineages);
Therapy-related myeloid neoplasms. AML and MDS, therapy related-Alkylating agent or radiation-related type; topoisomerase II inhibitor type; others;
AML, not otherwise classified-AML, with minimal differentiation; AML, without maturation; AML, with maturation; acute myelomonocytic leukemia; acute monoblastic or monocytic leukemia; pure erythroid leukemia; acute megakaryoblastic leukemia; acute basophilic leukemia; acute panmyelosis and myelofibrosis; myeloid sarcoma

### Current Barriers in AML Treatment

### Biological Properties of the AML Niche

Tumor escape strategies in AML involve direct adaptation of the AML cells to hide from immune recognition, and tumor-cell-mediated modifications of the immune cell compartment that include effector T cells, natural killer cells (NKs), and dendritic cells (DCs). [Tettamanti, S. et al. Leukemia (2022) 36 (1): 13-22, citing Guo, R. et al. Biomark Res. (2021) 9:1; Bailur, J K, et al. JCI Insight (2020); Bruck, O. et al. Blood Adv. (2020) 4:274-86; Chretien, A-S et al. Proc Ntl Acad. Sci. (2021) 118: c2020459118; Vadakekolathu, J. et al. Sci. Transl. Led. (2020) 12].

### Crosstalk Between AML Blasts and Cellular Components of the TME

FIG. 3 schematically shows immune escape strategies employed by AML blasts. AML blasts can hamper T- and NK-cell effector functions by aberrantly overexpressing inhibitory T-cell ligands (i.e. PD-L1, Gal-9, CD155, CD112, CD86) (1), or by releasing soluble forms of NKG2DL AML blasts promote T-cell exhaustion and apoptosis, drive the expansion of regulatory T cells (Tregs) and myeloid-derived suppressor cells (MDSCs), and induce the switch of macrophages to tumor-associated macrophages (TAMs) (3) by altering the cytokine milieu and through the release within the BM niche of other soluble factors such as reactive oxygen species (ROS), indoleamine 2,3-dioxygenase-1 (IDO1), arginase II (ArgII), and extracellular vesicles (EVs) (4). Moreover, AML blasts reduce their expression of antigen presentation molecules, thus hiding themselves from immune cells such as dendritic cells (DCs) and macrophages (5). Taken from Tettamanti, S. et al. Leukemia (2022) 36 (1): 13-22].

Gene expression profiling of AML blasts from patients relapsed after HSCT has uncovered transcriptional signatures enriched in altered immune-related processes, including the epigenetic downregulation of HLA class II genes, the genomic loss of HLA, the epigenetic upregulation of T cells inhibitory ligands, and the deregulated release of immunosuppressive molecules [Id., citing Toffalori, C. et al. Nat. Med. (2019) 25:603-11; Jan, M. et al. Blood Adv. (2019) 3:2199-204; Christopher, M J et al. N. Engl. J. Med. (2018) 379:2330-41; Stolzel, F. ct al. Transplantation (2012) 93:744-9].

Galectin-9, a ligand of T-cell immunoglobulin and mucin domain 3 (TIM-3), a co-inhibitory receptor that is expressed on IFN-γ-producing T cells, FoxP3+Treg cells and innate immune cells (macrophages and dendritic cells) where it has been shown to suppress their responses upon interaction with their ligand(s) [Das, M. et al. Immunol. Rev. (2017) 276 (1): 97-111]. is highly involved in creating an autocrine loop that seems essential in the maintenance of leukemic stem cells (LSCs) [Id., citing, Kikusige, Y. et al. Stem Cell (2010) 7:708-17; Kikushige, Y. et al. Oncology (2015) 89:28-32]. In AML murine models and in patients, there is a strong association between high frequency of TIM-3+ and PD-1+T cells and poor prognosis [Kong, Y. et al. Blood Cancer J. (2015) 5:330; Darwish, N H et al. Oncotarget (2016) 7:57811; Kamal, A M et al. Oncol. Lett (2021) 21:1-9; Jan, M. et al. Proc. Natl Acad. Sci. USA (2011) 108:5009-14]. TIM-3 is a well-defined Immune checkpoint in both effector T and NKcells. TIM-3 binds to galectin-9, which is highly expressed on AML blasts, and has been found to promote self-renewal via stimulatory β-catenin and NFκB-signaling, and to reduce the release of pro-inflammatory cytokines, ultimately resulting in NK- and T-cell dysfunction. Another inhibitory receptor, TIGIT (T-cell immunoglobulin and ITIM domain), which binds to the same ligands as DNAM-1, CD155, and CD112, also has been shown to be upregulated in AML blasts. Low levels of DNAM-1 expression are observed in AML patients, while its ligands are highly expressed [Id., citing Wang, M. et al. Clin. Immunol. (2018) 190:64-73; Gao, J. et al. Cancer Sci. (2017) 108:1934-8] suggesting that the binding of TIGIT with CD112 and CD155 ligands may represent a mechanism of tumor immune escape promoted by immune cell inhibitory signaling. This notion is further supported by clinical observations showing that CD112 and CD155 expression are associated with poor prognosis in AML [Id., citing Stamm, H. et al. Mamm Genome (2018) 29:694-702]. In another recent study, high mRNA levels of the inhibitory receptors Cytotoxic T-lymphocyte associated protein 4 (CTLA4) and lymphocyte activating-3 (LAG-3) in AML blasts were also shown to be predictive of an unfavorable prognosis [Id., citing Radwan, S. et al. Clin. Lymphoma Myeloma Leuk (2020) 20: S198].

AML blasts alter the formation of T-cell immune synapses. Gene expression profiling revealed an aberrant T-cell activation signature in AML patients. In particular, differentially expressed genes were reported to be involved in actin cytoskeletal formation, and correlative functional data demonstrated an impaired capacity of T cells in forming an immune synapse with AML blasts [Id., citing LeDieu, R. et al. Blood (2009) 114:39099-16]. Previous studies had shown that T cells isolated from AML patients are phenotypically effector cytotoxic T lymphocytes and express activation markers, but are impaired in their cytotoxic potential, meaning the capacity to express cytotoxic granules [Id., citing Lim, S H, et al. Leuk. Res. (1991) 15:641-4].

Functional alterations of T cells in AML are also a consequence of a dysregulated cytokine network directly mediated by AML blasts. Several studies have documented high numbers of Tregs in patients with AML [Id, citing Curti, A. et al. Blood (2007) 109:2871-7; Ersvaer, E. et al. BMC Immunol. (2010) 11:38]. In particular, Shenghui et al. [Shenghui, Z. et al. Intl J. Cancer (2011) 129:1373-81] showed that elevated frequency of CD4+CD25+CD127low/-Tregs in AML is associated with poor prognosis. Tregs enrichment in the AML niche has been associated with the capacity of AML blasts to secret immunoinhibitory factors, such as IL-10, IL-35, transforming growth factor-beta (TGF-β), and indoleamine 2,3-dioxygenase 1 (IDO1) [Id., citing Chen, W. et al. J. Exp Med. (2003) 198:1875-86; Walker, R M, et al. J. Clin. Invest. (2003) 112:1437-43; Cools, N. et al. Cell Mol. Med. (2008) 12:690-700]. These soluble factors push T-cell polarization towards induced Tregs promoting T-cell tolerance and leukemia progression. In particular, IDO1, which catabolizes the degradation of tryptophan to N-formylkynurenine and leads to a reduction in local tryptophan concentration and accumulation of toxic tryptophan metabolites cooperate that arrest T-cell proliferation., has been shown to correlate with poor prognosis [Id., citing Folgiero, V. et al. Oncotartet (2014) 5:2052-64]. Moreover, tryptophan-derived metabolites like L-kynurenine inhibit antigen-specific T-cell proliferation and induce T-cell apoptosis. This cytokine imbalance reduces the production of pro-inflammatory cytokines, such as IL-15 and interferon-gamma (IFN-γ), further propagating the negative effects on T-cell effector functions [Id., citing Binder, S. et al. Cytokine Growth Factor Rev. (2018) 43:8-15].

Other soluble factors related to different metabolic pathways have also been reported to modulate the TME in leukemia. For example, high levels of arginase II in the plasma of AML patients were shown to impair T-cell proliferation and to polarize monocytes toward an immunosuppressive M2-like phenotype. In addition, increased arginine metabolism inhibited the proliferation of hematopoietic progenitors, contributing to a wider suppressive TME [Id., citing Mussai, F. et al. Blood (2013) 122:749-58]. Together with arginine II, upregulation of the inducible nitric oxide synthase (iNOS) by AML blasts was reported to correlate to inhibition of T-cell proliferation, increase in Tregs, and decreased number of NKT cells [Id., citing Jacamo, R. et al. Blood (2017) 130:2443].

Furthermore, AML blasts can metabolize both glucose and fatty acids, released by surrounding stromal adipocytes, to derive acetyl-CoA to drive the Krebs cycle and oxidative phosphorylation (OXPHOS) for ATP production. LSCs in the AML niche express the fatty acid transporter CD36, and induce lipolysis in BM adipocytes to fuel fatty acid oxidation (FAO) in leukemic cells [Ye, H. et al. Cell Stem Cell (2016) 19:23-27].

### AML Blasts Escape NK Cell Recognition

Mechanisms of NK-cell evasion and escape by AML blasts include an altered expression of NK-cell ligands caused by epigenetic changes, such as incorrect hypermethylation of genes encoding ligands for the activating receptor NKG2D (NKG2DL), namely MICA, ULBP1, ULBP2, and ULBP3 genes [Id., citing Baragano, R A, et al. Genes Immun. (2015) 16:71-82]. NKG2DL-negative leukemic cells that escaped the NK-cell immune recognition were shown to have an immature morphology and molecular and functional stemness characteristics [Paczulla, A M et al. Nature (2019) 572:254-9]. Moreover, AML blasts were shown to release a soluble form of NKG2DL (sNKG2DL), through cleavage by metalloproteases or into exosomes, causing the downregulation of NKG2D receptor on NKs and impairing their cytotoxic activity. AML blasts also express high levels of ligands, such as CD112 and CD155, that cause a decrease in their activating receptor DNAM-1 on NKs, ultimately leading to an altered degranulation of NKs and impaired cytotoxic activity [Id., citing Costello, R T et al. Blood (2002) 99:3661-7; Sanchez-Correa, B. et al. Immunol. Cell Biol. (2012) 90:109-15]. AML blasts may also escape NKs by induction of co-inhibitory receptors in NKs that include TIGIT, which inhibits IFN-γ release [Id., citing Kong, Y. et al. Clin. Cancer Res. (2016) 22:3057-66]. High TIGIT expression at engraftment has been associated with a reduced number of NKs in the BM, reduced incidence of acute graft-versus-host disease, and poor survival [Id., citing Hattori N. et al. Biol. Blood Marrow Transpl. (2019) 25:861-7].

### AML Blasts Increase Myeloid-Derived Suppressor Cells and Tumor-Associated Macrophages

Myeloid-derived suppressor cells (MDSCs) cause T-cell tolerance through multiple mechanisms that include expression of V-domain Ig suppressor of T-cell activation (VISTA), PD-L1, IDO1, arginase, and production of reactive oxygen species (ROS), peroxynitrate, and multiple cytokines (TGF-β and IL-10) [Id., citing Yang, Y. et al. Front Immunol. (2020) 11: AML blasts can promote MDSCs expansion by releasing extracellular vesicles (EVs) containing the oncoprotein MUCI, which, in turn, increases c-myc expression in EVs through microRNA miR34a, leading to MDSCs proliferation [Id., citing Pyzer, A R et al. Blood (2017) 129:1791-801]. The Akt/mTOR pathway has been shown to play a critical role in the AML-EV-induced phenotypical and functional transition from monocytes to MDSCs. Monocytes engulfing AML-derived EVs acquire the CD14+HLA-DR^{low} inhibitory phenotype and upregulate expression of genes characteristic for MDSCs, such as S100A8/9 and cEBPB [Id., citing Tohumeken, S. et al. Cancer Res. (2020) 80:3663-76]. In AML patients, it has been reported that MDSCs were more abundant in BM and in peripheral blood (PB) compared with healthy controls [Id., citing Pyzer, A R et al. Blood (2017) 129:1791-801]. There is also an association between Tregs and MDSCs numbers in myelodysplastic syndrome (MDS), which correlates with a higher risk of transformation to AML, indicating a potential role for MDSCs in AML progression [Id., citing Kittang, A O et al. Oncoimmunology (2015) 5: e1062208].

Macrophages are critical cellular components of the immunosuppressive TME. The intrinsic plasticity of macrophages renders this cell subset particularly susceptible to tissue-specific regulation. Within the TME, tumor-associated macrophages (TAMs) are generally defined as M2 macrophages, and are characterized by anti-inflammatory activity by secreting arginase, metalloproteinases, TGF-β, IL-10, and other cytokines that cause immune suppression, angiogenesis and tissue repair [Id., citing Mantovani, A. et al. Trends Immunol. (2002) 23:549-55]. Al-Matary et al. reported that TAMs are elevated in the BM of AML patients compared to healthy donors. Moreover, AML blasts can directly drive TAMs to an M2-like phenotype in the BM and spleen of tumor-bearing mice [Id., citing Al-Matary, Y S, et al. Haematologica (2016) 101:1216-27].

### Stromal and Vascular Niches Promote Resistance to Immunotherapy

The TME in AML causes resistance to conventional chemotherapy and suppresses anti-tumor immune responses. Leukemia-associated remodeling within the AML niche, including changes associated with increased hypoxia and inflammation as well as metabolic reprogramming, facilitate immune evasion and activation of survival pathways favoring AML progression [Id., citing Mendez-Ferrer, S. et al. Nat. Rev. Cancer (2020) 20:285-98].

### The AML Niche Shows Altered Immune Cell Homing

CXCL12 expressed by BM stromal cells and its receptor CXC receptor 4 (CXCR4) play a key role in the migration of LSCs to the BM niche. High expression of CXCR4 on AML blasts has been shown to predict poor prognosis [Id., citing Spoo, A C et al. Blood (2007) 109:786-91]. The CXCL12/CXCR4 axis can also activate pathways that favor the survival, growth, and chemotherapy resistance of AML blasts [Cancilla, D. et al. Front. Oncol. (2020) 10:1672]. CXCL12 expression seemed to be reduced in MSCs in AML, fostering the migration of CXCR4-overexpressing malignant LSCs versus normal hematopoietic stem cells (HSCs) [Hanoun, M. et al. Cell Stem Cell (2014) 15:365-75]. CXCR4 is also involved in the trafficking of adoptively transferred lymphocytes or CAR T cells to the BM niche. Suppression in the ability of stromal cells to produce CXCL12 in the AML TME may dampen their migration and infiltration into the BM, as reported for other hematological malignancies [Id., citing Ponzetta, A et al. Cancer Res. (2015) 75:4766-77].

### Metabolic Alterations of the AML Niche

MSCs from AML patients have a higher propensity to differentiate into adipocytes, and the interaction between AML blasts and adipocytes in the BM niche creates a unique microenvironment that supports the metabolic demands of leukemia [Id., citing Ye, H. et al. Cell Stem Cell (2016) 19:23-37; Azadniv, M. et al. Leukemia (2020) 34:391-403]. AML blasts induce hormone-sensitive lipase in adipocytes and activate lipolysis, which then enable FABP4-dependent transfer of fatty acids to leukemia cells, thus enhancing fatty acid oxidation (FAO) [Id., citing Shafat, M S et al. Blood (2017) 129:1320-32; Tabe, Y. et al. Cancer Res. (2017) 77:1453-64]. Fatty acid abundance can hamper effector T-cell functions and promote Tregs differentiation [Id., citing Michalek, R D et al. J. Immunol. (2011) 186:3299-303]. In fact, FAO can inhibit the activation of effector T cells by increasing PD-1 expression and inhibiting IFN-γ secretion, while promoting Treg cell generation through activation of the MAPK signaling pathway. FAO has also a key role in polarizing M2 macrophages [Id., citing O'Neill, L A J et al. Nat. Rev. Immunol. (2016) 16:553-65].

MSCs can transfer mitochondria to AML cells through endocytic pathways or tunneling nanotubes (TNT), a process that is further boosted by chemotherapy and associated with increased oxidative phosphorylation-derived ATP production in the recipient cells [Id., citing Moschoi, R. et al. Blood (2016) 128:253-64]. AML-derived nicotinamide adenine dinucleotide phosphate oxidase-2 (NOX2) drives the transfer of mitochondria via the generation of superoxide [Id., citing Marlein, C R et al. Blood (2017) 130:1649-60]. Gap-junction interactions between AML cells and MSCs in the leukemic niche have been implicated in the regulation of leukemic cell metabolism [Id., citing Kouzi, F. et al. Oncogene (2020) 39:1198-212]. The constitutive activation of NOX and the mitochondrial production linked to OXPHOS (a fundamental mitochondrial process linking the TCA cycle to the production of ATP) are the primary sources of large amounts of ROS that are particularly abundant in AML of M4 and M5 subtypes [Id., citing Hole, P S et al. Blood (2013) 122:3322-30; Farge, T. et al. Cancer Disco. (2017) 7:716-35]. AML blasts can use ROS to evade anti-leukemic effector lymphocytes since free radicals inactivate T and NK cells by triggering PARP-1 dependent apoptosis [Id., citing Aurelius, J. et al. Blood (2012) 119:5832-7].

AML progression has been shown to cause significant remodeling of vascular endothelium mainly via nitric oxide (NO), with increased vascular permeability and decreased blood flow, which results in the formation of a hypoxic leukemia niche [Id., citing Passaro, D. et al. Cancer Cell (2017) 32:324-41.c6]. The endosteal BM region is the main site of this vessel loss [Id., citing Duarte, D. et al. Cell Stem Cell (2018) 22:64-77.e6]. As a consequence, several BM areas are hypoperfused and both drug biodistribution and immune cell trafficking are compromised [Id., citing Carmeliet, P. and Jain, RK. Nat. Rev. Drug Disco. (2011) 10:417-27; Rytelewski, M. et al. J. Immunother. Cancer (2019) 7:1-13]. Finally, the adhesive properties of the immune cells to the endothelium are also altered due to the increased levels of E-selectin induced by the inflammation generated by AML blasts [Id., citing Barbier, V. et al. Nat. Commun. (2020) 11:2042].

Unravelling the heterogeneity of AML at the clinical, cytogenetic, and molecule levels has allowed improved prognostic and predictive abilities and led to the development of selected therapies for AML subsets. Research efforts in the last decade have expanded the pathophysiologic-molecular subsets of AML, through identification of prognostic, predictive, and targetable molecular abnormalities [Kantarjian, H. et al. Blood Cancer J. (2021) 11:41, citing Patel, J P et al. N. Engl. J. Med. (2012) 366:1079-89; Cancer Genome Atlas Research Network, et al. N. Engl. J. Med. (2013) 368:2059-74; Ding, L. et al. Nature (2012) 481:506-10; Papaemmanuil, E. et al. N. Engl. J. Med. (2016) 3; 74:2209-21; Grimwade, D. et al. Blood (2010) 116:354-65; Pastore, F. et al. J. Clin. Oncol. (2014) 32:1586-94; Richard-Carpentier, G. and Di Nardo, CD Hematol. Am. Soc. Hematol. Educ. Program (2019) 548-56; Short, NJ ct al. Cancer Discov. (2020) 10:506-25]. Ongoing studies and recently approved agents in AML of particular interest include:
(1) Combinations of epigenetic therapy with hypomethylating agents (HMAs; azacitidine, decitabine) and venetoclax in older patients (or patients unfit for intensive chemotherapy); and combinations of intensive chemotherapy and venetoclax in younger/fit patients.
(2) The addition of fms-like tyrosine kinase 3 (FLT3) inhibitors (gilteritinib, midostaurin, sorafenib, quizartinib, crenolanib, others) to intensive chemotherapy, or to HMA/low-intensity therapy in FLT3-mutated AML.
(3) The addition of isocitrate dehydrogenase (IDH) inhibitors (IDH1 inhibitor ivosidenib; IDH2 inhibitor enasidenib) and/or venetoclax in IDH1/2-mutated AML.
(4) Investigations of the roles of APR246 (a TP53 modulator) and of magrolimab (an anti-CD47 monoclonal antibody enhancing macrophage-mediated phagocytosis) in TP53-mutated AML.
(5) Exploring the role of menin inhibitors in mixed-lineage leukemia (MLL1)-rearranged acute leukemia. Menin inhibitors inhibit a protein that is part of the menin KMT2A epigenetic complex. This complex is particularly active and pivotal for leukemia genesis related to NPM1-mutant and KMT2A-rearranged AML.
(6) Investigations of combined small-molecule targeted therapies, with or without standard intensive chemotherapy or hypomethylating agents (HMAs) (+/−venetoclax; at the expense of worsening myelosuppression), in order not only to prolong survival, but also to improve the potential cure rates in previously incurable AML subsets.
(7) Establishing maintenance therapy as an important strategy in AML (as it is in acute lymphoblastic leukemia [ALL]).
(8) Developing oral anti-AML therapy (e.g., oral decitabine, oral azacitidine) to replace and improve upon the effects of parenteral therapies.
(9) Approaches to enhance T-cell immune responses to AML (as done in ALL) with T-cell engagers (BiTEs), checkpoint inhibitors, and chimeric antigen receptor (CAR)-T-cell approaches. [Id].

Measurable residual disease (MRD) in complete remission. Measuring residual disease in AML in complete remission (CR) is now part of the standard of care in AML [Id., citing Jongen-Lavrencic, M. ct al. N. Engl. J. Med. (2018) 378:1189-99; Grimwade, D. and Freeman, SD. Blood (2014) 124:3345-55; Pastore, F. & Levine, R L. JAMA (2015) 314:778-80; Kico, J M et al. JAMA (2015) 314:811-22; Ravandi, F. et al. Cancer (2017) 123:426-35; Short, N J et al. JAMA Oncol. (2020) 6:1890-99; Hourigan, C S et al. J. Clin. Oncol. (2020) 38:1273-83]. The detection of MRD at the time of morphologic CR is associated with a higher relapse rate and with worse survival in AML.

Younger Patients with AML (and/or Older Patients Fit for Intensive Chemotherapy)

The median age of patients with AML is 68 years [Id., citing Sasaki, K. et al. Cancer (2021) 127 (12): 2049-61]. Most of the experience with 3+7 (3 days of daunorubin +7 days of cytarabine) and other intensive chemotherapy regimens was conducted in younger patients, usually with an upper age limit 60-65 years.

The optimal frontline therapy for younger patients with AML is evolving. An increasing body of research suggests that there are better induction-consolidation regimens than 3+7. Modifications of frontline AML therapy include: (1) The use high-dose cytarabine combination during induction. (2) Optimization of the dose of daunorubicin (60 mg/m2 daily ×3, versus 45 mg/m2 or 90 mg/m2 daily ×3) and the use of other anthracyclines (e.g., idarubicin, mitoxantrone). (3) The addition of adenosine nucleoside analogs (fludarabine, clofarabine, cladribine) to cytarabine-anthracyclines. (4) The addition of the CD33-targeted monoclonal antibody gemtuzumab ozogamicin (GO). (5) The addition of targeted therapies such as FLT3 and IDH inhibitors in appropriate patients. (6) The addition of the BCL-2 inhibitor venetoclax to induction therapy on investigational trials. (7) The use of maintenance therapy with oral azacitidine. [Id.]

Older Patients with AML (or Younger Patients not Fit for Intensive Chemotherapy.

Older patients with AML tolerate intensive chemotherapy poorly. In a study by Lowenberg and colleagues [Id., citing Lowenberg, B. et al. N. Engl. J. Med. (2009) 361:1235-48] evaluating 3+7 with daunorubicin 45 mg/m2 versus 90 mg/m2 daily ×3, among 813 selected patients 60 years and older (median age 67 years), the median survival was 7 to 8 months and the estimated 3-year survival rate was 20%. The study reported an acceptable low early mortality rate of 11-12%.

The treatment of older patients with AML remains challenging. Acute myeloid leukemia in older patients carries a distinctly different disease biology associated with high risk and often complex karyotype, a high incidence of cytogenetic abnormalities involving monosomies 5 and 7 and chromosome 17 abnormalities, a high incidence of multiple mutations including TP53 (20+%), and a high incidence of secondary/therapy-related AML (20 to 30%). Older patients have multiple co-morbidities (hypertension; diabetes; organ dysfunctions including cardiac, pulmonary and renal abnormalities) that result in poor tolerance to intensive chemotherapy and high early (4- to 8-week) mortality rates. In community practice (SEER data; 2010-2017) treating unselected older patients, the 4-week mortality was 24% among patients 60-69 years old and the 5-year survival 18%. Among patients 70 years and older (45% of all AML), the 4-week mortality was 44% and the 5-year survival 4%. [Id].

Faced with the poor results with intensive chemotherapy, investigators began in the 1990's evaluating lower-intensity strategies in patients unfit for intensive chemotherapy (expected high-early mortality). These included low-dose cytarabine, HMA therapy, and targeted therapies (monoclonal antibodies; more recently FLT3 inhibitors and IDH 1/2 inhibitors). Over time and over several investigational studies since 2000, lower-dose chemotherapy/HMA therapy combinations have been shown to provide, since 2015, overall response rates as high as with intensive chemotherapy, significantly lower rates of early mortality and myclosuppression-associated complications, and survival equivalent or superior to intensive chemotherapy [Id., citing Quintas-Cardama, A. ct al. Blood (2012) 120:4840-45]; Takahashi, K. ct al. Clin. Lymphoma Myeloma Leuk. (2016) 16:163-8 c1-2].

The search for ligands and receptors involved in the promotion or inhibition of AML cell growth, progression and susceptibility to cytotoxic drugs is still ongoing.

### IV. B Cell Maturation Antigen (BCMA): A Promising Therapy for Multiple Myeloma, Another Hematological Cancer

Multiple myeloma (MM) is a hematological cancer characterized by the accumulation of neoplastic plasma cells in the bone marrow associated with elevated serum and/or urine monoclonal paraprotein levels During the course of the disease, patients with MM usually suffer debilitating clinical manifestations linked, directly or indirectly to the accumulation of tumor plasma cells, including lytic bone lesions, anemia, immunodeficiency and renal function impairment. [Tonon, G. and Anderson, KC. Chapter 30, Multiple Myeloma, in The Molecular Basis of Cancer, Mendelsohn, J., Gray, JW, Howley, P M, Israel, MA, and Thompson, CB. Elsevier Saunders; (2015), pp. 455-66]. MM is almost always preceded by a condition termed monoclonal gammopathy of undetermined significance (MGUS) [Id. citing Landagren, O. et al. Blood (2009) 113:5412-7; Weiss, B M et al. Blood (2009) 113:5418-22], defined by the presence in the serum of a monoclonal paraprotein below 30 g/L, an accumulation of less than 10% plasma cells in the bone marrow, in the absence of clinical manifestations. MGUS is present in 1% of adults over age 25 and evolves toward malignant MM at a rate of 0.5-3% per year [Id., citing Kyle, R A, et al. Leukemia (2010) 24:1121-27; Zingone, A. and Kuchl, WM Semin. Hematol. (2011) 48:4-12].

Unlike other hematological cancers such as leukemias and lymphomas that present a relatively unscathed karyotype, the MM genome is thoroughly reshuffled. The wide array of genetic lesions that have been described in MM include chromosomes gains or losses, Ig-related chromosomal translocations, gains or losses or small chromosomal segments, and genetic and epigenetic modifications affecting single genes. Approximately half of patients present a hyperdiploid (HD) karyotype (number of chromosomes ranging from 48 to 74 compared to a normal human karyotype of 46 chromosomes) with concomitant gains of several odd chromosomes (such as 3, 5, 7, 9, 11, 15, 19, or 21, in various combinations). The remaining patients belong to the nonhyperdiploid (NHD) group, which includes cases with hypodiploid, pseudodiploid, near-diploid or tetraploid karyotypes [Id. citing Steinman, R M and Dhodapkar, M. Int. J. Cancer (2001) 94:459-73]. At the MGUS stage, patients present either an HD or an NHD karyotype [Id., citing Chng, W J et al. Blood (1005) 106:2156-61], which is maintained during the progression of the disease. [Id., citing Chng, W J et al. Leuk Res. (2006) 30:266-71].

MM specific chromosomal translocations, so called primary translocations present from the MGUS phase [Id.,citing, deJong, E C et al. J. Immunol. (2002) 168:1704-9], involve the immunoglobulin H (IgH) locus (at 14q32.3 [Id., citing van Duin, D. et al. Trends Immunol. (2006) 27:49-55] and less frequently the IgL locus (2p12, kappa or 22q11, lambda [Id citing van Duin, D. et al. Trends Immunol. (2006) 27:49-55], and juxtapose strong Ig enhancers to various genes, resulting in their increased expression. These primary translocations are for the most part confined to the NHD group. [Id., citing Fonseca, R. et al. Blood (2003) 102:2562-67]

Over this general framework, additional genetic or epigenetic modifications are present in the MM genome. For example, deletions affecting tumor suppressor genes, such as TP53, UTX, FAM46C, and the NFκB family members BIRC2, BIRC3, and CYLD, or focal amplifications of areas including oncogenes, such as MYC, HGF, MCLI and IL6R, have been reported. In the case of MM, four pathways were significantly enriched in somatic mutations; genes belonging to the NFκB pathway were frequently mutated; frequent mutations affecting histone-modifying genes, such as MLL, MLL2, MLL3, UTX, MMSET, and WHSCILI were reported. Mutations affecting the same nucleotide were also found in the IRF4 transcription factor and its target PRMDI, confirming previous functional data reporting a prominent role of IRF4 for MM survival. [Id., citing Shaffer, A L et al. Nature (2008) 454:226-31].

MM cells utilize the physiological mechanisms underlying healthy plasma cell homing to the bone marrow and the pathways supporting long-lived plasma cells. MM plasma cells establish tight interactions with essentially all the BM components; the BM microenvironment includes an ECM (collagen, laminin, fibrtonectin, and osteopontin) and a rich cellular component. The cellular BM compartment consists of hematopoietic and mesenchymal progenitor and precursor cells, including hematopoietic stem cells (HSCs), bone marrow-derived circulating endothelial precursors (CEPs) and endothelial cells (BMECs); immune cells (dendritic cells; B and T lymphocytes, NKT and NK cells, monocytes and macrophages); erythrocytes; megakaryocytes and platelets; and nonhematopoietic cells, including an ill-defined group of cells labeled fibroblasts/bone marrow stromal cells (BMSCs). Also included are cells involved in bone homeostasis, such as chondroclasts, osteoclasts (OCs) and osteoblasts (OBs). MM cells interact with BMSCs and the ECM either directly, via adhesion molecules that include LFA1, VLA4, NCAM, ICAMI and CD44, or indirectly through chemokines, cytokines and growth factors released by tumor cells and BMSC, such as IL-6, IGF-1, TNF-α, TGFβ1 and VEGF. As a result of these multiple layered interactions, several cancer-relevant pathways become activated in both the tumor and stromal cells, such as NFκB, JAK/STAT, P13K and MAPK that further increase MM growth and survival. The homing of MM cells to the BM also triggers a strong angiogenic response. [Id., Gabrilovich, D I et al. Clin. Cancer Res. (1999) 5:2963-70, Vacca, A. and Ribatti, D. Leukemia (2006) 20:193-9].

Smoldering/indolent multiple myeloma (SMM) is an asymptomatic condition defined by the presence of a serum monoclonal (M) protein of ≥3 g/dL and/or 10% to 60% clonal bone marrow plasma cells (BMPCs) with no evidence of end-organ damage (i.e., CRAB criteria) or other myeloma defining events. [Rajkumar, S V et al. Blood (2015) 125 (20): 3069-757]. It is distinguished from MGUS on the basis of the level of serum M protein and the percentage of clonal BMPCs. The disease definition of SMM was recently updated to exclude patients with BMPCs of ≥60%, serum involved/uninvolved free light chain (FLC) ratio of ≥100, and those with 2 or more focal lesions (typically indicating focal bone marrow abnormalities) on magnetic resonance imaging (MRI). [Id., citing Rajkumar, S V et al. Lancet Oncol. (2014) 15 (12): c538-48] Such patients have an approximately 40% per year risk of progression and are now considered to have MM. [Id., citing Rajkumar, S V et al. N. Engl. J. Med. (2011) 365 (5): 474-5; Kastritis, E. et al. Leukemia (2013) 27 (4): 947-53; Larsen, J T, et al. Leukemia (2013) 27 (4): 941-6; Waxman, A J et al. J. Clin. Oncol. (2014) 32 (5s) abstract 8607; Hillengass, J. et al. J. Clin. Oncol. (2010) 28 (9): 1606-10; Kastritis, E. et al. Leukemia (2014) 28 (12): 2402-3]. Light-chain SMM is a subtype of SMM in which there is monoclonal FLC excess with no expression of immunoglobulin heavy chain [Kyle, R A et al. N. Engl. J. Med. (2006) 354 (13): 1362-9; this entity is characterized by excess secretion of monoclonal FLC in the urine (Bence Jones proteinuria).

During the course of MM, patients relapse or become refractory to proteasome inhibitors (e.g., bortezomib, carfilzomib, ixazomib), immunomodulatory agents (e.g., thalidomide, lenalidomide, pomalidomide [Kleber, M. et al. J. Clincal Med. (2021) 10:4088, citing Siegel, R L et al. C A Cancer J. Clin. (2020) 70:7-30], and monoclonal antibodies, resulting in a very poor prognosis with a medium overall survival (OS) of 5.6 months [Id., citing Attal, M. et al. Lancet (201 (394:2096-2017; Ghandi, U H et al. Leukemia (201 ( ) 33:2266-75], especially in those patients with high-risk cytogenetics or failure to reach negative minimal residual disease [Id., citing Chim, C S et al. Leukemia (2018) 32:252-62; Munshi, N C et al. JAMAA Oncol. (2017) 3:28-35; Kastritis, E. et al. Clin. Lymphoma Myeloma Leuk. (2020) 20:445-52]. Although new generations of these drugs have become available, the management of these patients remains challenging for clinicians [Id., citing Ntassnasis-Stathopoulos, I. et al. Clin. Lymphoma Myeloma Leuk. (2021) 21:379-85].

### B Cell Maturation Antigen (BCMA) in MM

BCMA is a member of the tumor necrosis factor (TNF) receptor (TNFR) superfamily [Yu et al. J. Hematol. & Oncology. (2020) 13: article 126. Doi.org/10.1186/s13045-020-00962-7, citing Madry, C. et al. (1998) 10 (11): 1693-702]. It is encoded by a 2.92-kb TNFRSF17 gene located on the short arm of chromosome 16 (16p13.13) and composed of 3 exons separated by 2 introns. The BCMA gene product is a 184 amino acid and 20.2-kDa type III transmembrane glycoprotein, with the extracellular N terminus containing a conserved motif of 6 cysteines [Id., citing Kozlow, E J et al. Blood (1993) 81 (2): 454-61; Laabi, Y. et al. Nucleic Acids Res. (1994) 22 (7): 1147-54; Laabi, Yl et al. EMO J. (1992) 11 (11): 3897-904; Zhou, L J et al.].Immunol./(1992) 149 (2): 735-42]. There are four natural splice variants of human BCMA that present with different receptor binding affinities, membrane-anchoring ability, and intracellular domain signaling [Id., citing Laabi, Y. et al. Nucleic Acids Res. (1994) 22 (7): 1147-54, 23].

BCMA has binding sites for TNF associated factors (TRAFs) 1, 2 and 3 in its cytoplasmic tail and is capable of activating NF-κB, Elk-1, p38 MAPK, and JNK signaling pathways. [Gardam, S. and Brink, R. Front. Immunol. (2014) 4: article 509]. TRAFI is a signaling adaptor that plays a key role in pro-survival signaling downstream of TNFR superfamily members such as TNFRI, LMP1, 4-1BB and CD40. TRAF-2 typically signals cell survival through NF-κB and JNK activation and has been implicated in activation and negative regulation of the noncanonical NF-κB pathway. [Shi, J-H and Sun, S-C, Front. Immunol. (2018) 9:1849]. The adaptor protein TRAF3 serves as a negative regulator in multiple aspects of B cell biology. [Bishop, G A et al. Front. Immunol. (2018) 9:2161]. TRAF2-deficient B cells appear to have increased levels of TRAF3, indicating that TRAF2 helps target TRAF3 for ubiquitination and degradation.

BCMA, along with the other two functionally related TNFR superfamily members, B cell activating factor receptor (BAFF-R) and transmembrane activator and calcium modulator and cyclophilin ligand interactor (TACI), coordinate to regulate B cell proliferation maturation and survival, as well as differentiation into plasma cells (PCs) [Yu et al. J. Hematol. & Oncology. (2020) 13: article 126. Doi.org/10.1186/s13045-020-00962-7, citing Coquery, C M and Erickson, LD. Crit. Rev. Immunol. (2012) 32 (4): 287-305; Marsters, S A, et al. Curr. Biol. (2000) 10 (13): 785-88; Gross, J A, et al. Nature (2000) 404 (6781): 995-99; Thompson, J S et al. J. Exp. Med. (2000) 192 (1): 129-135; Sasaki, Y. et al. J. Immunol. (2004) 173 (4): 2245-52; Seshasayee, D. et al. Darce, J R et al. J. Immunol. (2007) 178 (9): 5612-22]. Unlike BAFF-R and TACI, BCMA is almost exclusively expressed on plasmablasts [Id., citing Avery, D T et al. J. Clin. Invest. (2003) 112 (2): 286-97] and PCs [O'Connor, B P et al. J. Exp. Med. (2004) 199 (1): 91-98]. It is also weakly detectable on some memory B cells committed to plasma cell differentiation and on plasmacytoid dendritic cells [Id., citing Tai, Y T et al. Blood (2014) 123 (20): 3128-38]. BCMA is undetectable in naïve B cells, hematopoietic stem cells, or in normal non-hematologic tissues except for some organs such as the testis, trachea, and some portions of gastrointestinal duct due to the presence of plasma cells [Id., citing Carpenter, R O et al. Clin. Cancer Res. (2013) 19 (8): 2048-60].

The upregulation of BCMA is induced by B lymphocyte-induced maturation protein 1 (Blimp-1), an essential transcription factor involved in the development and survival of plasma cells [Id., citing Deng, S. et al. Mol. Biol. Rep. (2010) 37 (8): 3747-55]. In BCMA−/−mice, the long-term survival of plasma cells is impaired. Lack of BCMA has no effect in short-lived plasma cells, B cell development, or early humoral immune response, and the splenic architecture and germinal centers appear intact in these BCMA-deficient mice [Id., citing O'Connor, B P et al. J. Exp. Med. (2004) 199 (1): 91-98; Xu, S. and Lam, KP. Mol. Cell Biol. (2001) 21 (12): 4067-74].

BCMA is identified on the surface of nearly all MM cell lines (80-100%) and is more abundantly present in malignant PCs than normal PCs [Id., Lee, L. et al. Br. J. Haematol. (2016) 174 (6): 911-22; Eckhert, E. et al. Immunotherapy (2019) 11 (9): 801-11]. MM patients receiving allogeneic transplant often develop donor-derived anti-BCMA monoclonal antibodies (mAbs) after donor lymphocyte infusion and benefit from a graft-versus-tumor response [Id., citing Bellucci, R. et al. Blood (2005) 105 (10): 3945-50]. In contrast, TACI is expressed at a significantly lower concentration and BAFF-R is even hardly detectable on MM cells [Id., citing Claudio, J O et al. Blood (2002) 100 (6): 2175-86]. BCMA overexpression significantly promotes in vivo growth of xenografted MM cells in murine models [Id., citing Tai, Y T, et al. Blood (2016) 127 (25): 3225-36]. Furthermore, BCMA expression is upregulated during MM pathogenesis and evolution, from normal to MGUS to SMM to active MM [Darce, J R et al. J. Immunol. (2007) 179 (11): 7276-86]. Higher levels of BCMA are associated with poorer outcomes [Id., citing Lee, L. et al. Br. J. Haematol. (2016) 174 (6): 911-22], indicating that BCMA is a useful biomarker of disease activity and prognosis for MM.

BCMA has two agonist ligands: A Proliferation-Inducing Ligand (APRIL) and B-cell Activating Factor (BAFF; also called BLyS), which are mainly secreted by bone marrow (BM) stromal cells, osteoclasts, and macrophages in a paracrine manner in the BM [Id., citing Tai, Y T, et al. Blood (2016) 127 (25): 3225-36; Moreaux, J. et al. Blood (2004) 103 (8): 3148-57; Tai, Y T et al. Cancer Res. (2006) 66 (13): 6675-82; Mulazzani, M. et al. J. Hematol. Oncol. (2019) 12 (1): 102] (FIG. 4). APRIL exhibits a much higher binding affinity to BCMA than does BAFF [Id., citing Day, E S et al. Biochemistry (2005) 44 (6): 1919-31], and it also binds to TACI [Id., citing Moreaux, J. et al. Eur. J. Haematol. (2009) 83 (2): 119-29], while BAFF restricts more selectivity to BAFF-R [Id., citing Day, E S et al., Biochemistry (2005) 44 (6): 1919-31]. Thus, APRIL is more specific to plasma cells and correlates to more downstream pathophysiological activities [Id., citing Bolkun, L. et al. Ann. Hematol. (2014) 93 (4): 635-44].

MM cell lines had significantly reduced growth when xenografted in APRIL−/−mice [Id., citing Matthes, T. et al. Leukemia (2015) 29 (9): 1901-8]. In MM patients, the serum levels of APRIL and BAFF are elevated about 5-fold over those in the healthy controls [Id., citing Moreaux, J. ct al. Blood (2004) 103 (8): 3148-57], and the more advanced the stage of MM is, the higher the concentration of ligands that is detected [Id., citing Pan, J. et al. Oncol. Lett (2017) 14 (3): 2657-62]. Studies have shown that MM cells can stimulate osteoclasts to produce more APRIL which contributes to an immunosuppressive BM microenvironment [Tai, Y T et al. Blood (2016) 127 (25): 3225-36; Yaccoby, S. et al. Leukemia (2008) 22 (2): 406-13].

Upon binding of the ligands to BCMA, multiple growth and survival signaling cascades are activated in MM cells, most frequently nuclear factor κ-light-chain enhancer of activated B cells (NF-κB), but also including rat sarcoma/mitogen-activated protein kinase (RAS/MAPK), and phosphoinositide-3-kinase-protein kinase B/Akt (PI3K-PKB/Akt) signaling pathways [Id., citing Eckhert, E. et al. Immunotherapy (20019) 11 (9): 801-11; Demchenko, Y N, et al. Blood (2010) 115 (17): 3541-52; Hua, H. et al. J. Hematol. Oncol. (2019) 12 (1): 71]. These pathways result in proliferation stimulation by modulating cell cycle checkpoints, increased survival by upregulating anti-apoptotic proteins (e.g., Mcl-1, BCL-2, BCL-XL), and production of cell adhesion molecules (e.g., ICAM-I), angiogenesis factors (e.g., VEGF, IL-8), and immunosuppressive molecules (e.g., IL-10, PD-L1, TGF-β) [Id., citing Eckhert, E. et al. Immunotherapy (2019) 11 (9): 801-11; Tai, Y T, et al. Blood (2016) 127 (25): 32225-3236; Tai, Y T, and Anderson, DC. Expert Opin. Biol. Ther. (2019) 19 (11): 1143-56]. In vitro studies have shown that BCMA overexpression can even trigger the activation of NF-κB and MAPK pathways in MM cells itself without stimulation of APRIL or BAFF [Id., citing Hatzoglou, A. ct al. J. Immunol. (2000) 165 (3): 1322-30]. In addition, there are many cross-talks between APRIL/BCMA signaling and other pathways. For example, APRIL interacts with CD138/syndecan-1 and heparan sulfate proteoglycans (HSPG) to promote proliferation and survival of MM cells [Hendriks, J. et al. Cell Death Differ. (2005) 12 (6): 637-48]. Concomitant blockade of FGF-R3 and JAK2 leads to BCMA downregulation [Cassinelli, G. et al. Biochem. Pharmacol. (2009) 78 (9): 1139-47]. In vitro studies have shown that BCMA co-immunoprecipitates with interferon regulatory factor-4 (IRF-4), a master transcription factor mediating survival of MM cells, further emphasizing its role in the oncogenesis of MM [Id., citing Shaffer, A L et al. Nature (2008) 454 (7201): 226-31].

Soluble BCMA (sBCMA)

BCMA has a soluble form, sBCMA, derived from direct shedding of the membrane BCMA through γ-secretase activity. sBCMA retains the extracellular domain and a part of the transmembrane region of the molecule [Id., citing Laurent, S A et al. Nature Commun. (2015) 6: 7335]. sBCMA represents a potential biomarker for B cell involvement in human autoimmune diseases such as systemic lupus erythematosus, rheumatoid arthritis, and multiple sclerosis [Id., citing Laurent, S A et al. Nat. Commun. (2015) 6:7333; Gutierrez, et al. J. Immunol. Res. (2019) 2019:3658215]. In MM patients, the serum level of sBCMA is found to be significantly elevated compared to healthy individuals [Id., citing Sanchez, E. et al. Br. J. Haematol. (212) 158 96]: 727-38]. A higher serum level of sBCMA independently correlates to a heavier disease burden, a worse clinical and radiological response, and a poorer prognosis [Id., citing Ghermezi, M. et al. Haematologica (2017) 102 (4): 785-95]. In patients with good responses to BCMA-targeted immunotherapy, a remarkable decrease in sBCMA level was observed, suggesting sBCMA as a new biomarker for monitoring response to MM therapy [Id., citing Ali, S A et al. Blood (2016) 128 (13): 1688-700].

Unlike sBCMA, many studies have shown that the level of cell-surface BCMA does not seem to affect the response to BCMA-targeted immunotherapy [Id., citing Dettman, E J et al]. It has been suggested that the varied levels of surface BCMA seen in MM patients are simply the result of the shedding variations of the membrane BCMA, while high levels of sBCMA may interfere with BCMA-targeted immunotherapy by reducing the total amount of cell-surface BCMA and sequestering circulating ligands or anti-BCMA antibodies, thereby inhibiting efficient binding to MM cells [Id., citing Sanchez, E. et al. Clin. Cancer Res. (2016) 22 (13): 3383-97; Chen, H. et al. Leuk. Res. (2019) 81:62-6].

A γ-secretase inhibitor (GSI, LY3039478/JSMD194) was reported to decrease sBCMA concentration, while increasing cell-surface BCMA expression concurrently in MM cell lines, patient tumor cells, in murine models. This inhibitor significantly improved in vitro tumor recognition and in vivo anti-tumor efficacy of BCMA-specific chimeric antigen receptor (CAR)-T cells. Preclinical study has also discovered that short-term GSI administration to MM patients markedly increased the percentage of BCMA+ tumor cells [Id., citing Pont, M J et al. Blood (2019) 134 (19): 1585-97]. According to wwww.clinicaltrials.gov (visited Mar. 16, 2023), a phase 1 clinical trial (NCT03502577) to evaluate the safety and efficacy of combining CAR-T therapy with GSI, as well as cyclophosphamide (CTX), and fludarabine (FAMP) to treat patients with relapsed or persistent MM has been suspended pending additional funding.

### BCMA-Targeted Immunotherapy as a Therapeutic Strategy for MM

During the course of MM, patients relapse or become refractory to proteasome inhibitors (e.g., bortezomib, carfilzomib, ixazomib), immunomodulatory agents (e.g., thalidomide, lenalidomide, pomalidomide [see Kleber, M. et al. J. Clinical Med. (2021) 10:4088, citing Siegel, R L et al. C A Cancer J. Clin. (2020) 70:7-30], and monoclonal antibodies, resulting in a very poor prognosis with a medium overall survival (OS) of 5.6 months [Id., citing Attal, M. et al. Lancet (201 (394:2096-2017; Ghandi, U H et al. Leukemia (201 ( ) 33:2266-75], especially in those patients with high-risk cytogenetics or failure to reach negative minimal residual disease [Id., citing Chim, C S et al. Leukemia (2018) 32:252-62; Munshi, N C et al. JAMAA Oncol. (2017) 3:28-35; Kastritis, E. et al. Clin. Lymphoma Myeloma Leuk. (2020) 20:445-52]. Although new generations of these drugs have become available, the management of these patients remains challenging for clinicians [Id., citing Ntassnasis-Stathopoulos, I. et al. Clin. Lymphoma Myeloma Leuk. (2021) 21:379-85].

BCMA is a biomarker for the diagnosis of MM and for monitoring disease progression and treatment response even in non-secretory MM patients for whom no accurate monitoring for therapeutic efficacy has been available. Id., citing Demel, I. et al. Br. J. Haematol. (2021) 193:705-22].

Because BCMA is restrictively expressed in both normal and malignant plasma cells at high levels, it also makes sense as a target antigen for B cell neoplasms, including MM therapies. [Yu, et al. J. Hematology & Oncology (2020) 13:125, citing Lee, L. et al. Br. J. Haematol. (2016) 174 (6): 911-22; Wei, J. et al. J. Hematol. Oncol. (2019) 12 (1): 62]. Early studies on anti-BCMA antibodies showed robust cytotoxic activity against MM cells in vitro [Id., citing Ryan, M C et al. Mol. Cancer Ther. (2007) 6 (11): 3009-18].

Multiple BCMA-targeted treatment modalities, including antibody-drug conjugates (ADC), CAR-T cells, and bispecific T cell engagers (BiTEs), are under active clinical development for multiple myeloma [Id., citing Wu, C. et al J. Hematol. Oncol. (2019): 12 (1): 120; Liu, Q. et al. Mol. Cancer (2019) 18 (1): 154; Liu, D. et al. J. Hematol. Oncol. (2019) 12 (1): 15; Lonial, S. et al. Lancet Oncol. (2020) 21 (2): 207-21; Raje, N. et al. N. Engl. J. Med. (2019) 380 (18): 1726-37; Topp, M. et al. J. Clin. Oncol. (2019) 37 (no. 15_suppl): 8007-8007; Topp, M. et al. Blood (2018) 132 (Suppl. 1): 1010; Trudel, S. et al. Lancet Oncol. (2018) 19 (12): 1641-53; Zhao, W H et al., Blood (2018) 132 (Suppl. 1): 955; Zhao, W H et al. J. Hematol. Oncol. (2018) 11 (1): 141; Herrera, A F and Molina, A. Clinical Lymphoma, Myeloma and Leukemia (2018) 18 (7): 452-68.c454].

### Antibody-Drug Conjugates (ADC)

ADCs are among the fastest growing agents in plasma cell malignancies [Kleber, M. et al. J. Clincal Med. (2021) 10:4088., citing Braunstein, M. et al. Expert Rev. Hematol. (2021) 14:377-89; Demel, I. et al. Br. J. Haematol. (2021) 193:705-22]. Due to binding of their monoclonal antibodies (mAbs) to specific antigens on tumor cells, a sparing of normal cells and a minimizing of systemic toxicity can be achieved. This modality is limited by corneal toxicity.

### Anti-BCMA/CD3 Bispecific Antibodies

Bispecific antibodies (BsAbs) are molecules with affinities for two different epitopes capable of monovalent or bivalent binding on MM cells and on the CD38 on T cells [Id., citing Suurs, F V; et al Pharmacol. Ther. (2019) 201:103-19]. The strategy of therapy is that BsAbs, integrating a CD3 T cell receptor binding and tumor-binding domain, are linked to tumor cells via T cells and thus create an immunological synapse, leading to a release of granzymes and perforin and inducing lysis of the target cell [Id., citing Offner, S. et al. Mol. Immunol. (2006) 43:763-71]. The activated T cells release interferon-y and additional cytokines such as interleukin-6, -10 and tumor necrosis factor α, potentially inducing a cytokine release syndrome (CRS) with flu-like side effects such as fever, fatigue or headache in most patients [Id., citing Demel, I. et al. Br. J. Haematol. (2021) 193:705-22]. As of 2021, several bispecific T cell engagers (BITEs) targeting BCMA on MM cells and CD3 receptors on T cells were under investigation. This modality is limited by its short lifetime, which requires prolonged iv infusion time via central venous access.

### Anti-BCMA CAR-T Cells in MM

A CAR strategy targeting BCMA combines the characteristics of combining target specificity of mAbs and the cytotoxicity of T cells. The main advantage of CAR-T cells is that they, in contrast to human leukocytes antigen (HLA)-restricted T cell receptors, are not HLA-restricted, leading to an HLA type independent therapy strategy [Id., citing Mikkilineni, L. and Kochenderfer, JN. Blood (2017) 130:2594-2602]. Multiple clinical trials have shown promising therapeutic efficacy of CAR-T cells in patients with relapsed/refractory B cell neoplasms [Id., citing Maud, S L et al. N. Engl. J. Med. (2014) 371:1507-17; Lee, D W et al. Lancet (2015) 385:517-28; Maude, S L et al. N. Eng. J. Med. (2018) 378:439-48; Kochendorfer, J N et al. J. Clin. Oncol. (2015) 33:540-49]. Processes include CAR-T cells being produced after leukapheresis of peripheral white blood cells of the patients or healthy donors to obtain CD3+T cells for autologous vs. allogeneic CAR-T cells. Thereafter, the collected white blood cells, following a stimulation of T cells, express CD3 and CD28 or 4-1-BB by coated beads with mAb [Id., citing Nishida, H. Cancers (2021) 13:2712]. In a subsequent step, these activated T cells are transduced with a gene via a lentiviral vector, which encodes a receptor to the tumor-specific antigen on tumor cells, and are able to express CAR genes [Id., citing Nishida, H. Cancers (2021) 13:2712]. The main characteristic of CAR-T cells is that they use a CAR against antigen of tumor cells such as BCMA, CD19, CD138 (syndecan-1), Orphan G-protein-coupled receptor class C group 5 member D (GPRC5D) and immunoglobulin kappa light chain [Id., citing Garfall, A L et al. N. Engl. J. Med. (2015) 373:1040-7; Atamaniuk, J. et al. Eur. J. Clin. Investig. (2012) 42:953-60; Ramos, C A et al. J. Clin. Investig. (2016) 126:2588-96]. The role of BCMA in the pathogenesis of plasma cell malignancies offers a novel and attractive strategy for CAR-T cells [Id., citing Carpenter, R O et al Clin. Cancer Res. (2013) 19:2048-60].

TABLE 1
taken from Kleber, M. et al. J. Clinical Med. (2021) 10: 4088, summarizes
some of the agents then under clinical development for MM.

| Antibody-Drug | T cell Redirecting | |
|---|---|---|
| Conjugates | Bispecific Antibodies | CAR-T cell Therapy |
| Belantamab mafodotin | AMG-420 (formerly BI- | Idecabtagene Vicleucel (bb2121) |
| (humanized afucosylated | 836909) - BITE targeting | second generation BCMA- |
| anti-BCMA IgG1 | BCMA and CD3ε [Id., citing | targeted CAR-T cell |
| | Hipp, S. et al. Leukemia (2017) | incorporating a ScFv, 3-1BB co- |
| | 31: 1743-51] | stimulating domain and a CD3 |
| | | zeta signaling domain; induces |
| | | cytotoxicity against MM cells |
| | | independently of BCMA |
| | | expression or soluble BCMA [Id., |
| | | nishida, H. Cancers (2021) 13: |
| | | 2712; Raje, N. et al. N. Engl. J. |
| | | Med. (2019) 380: 1726-37] |
| AMG 224: anti-BCMA IgG | AMG-701 - next generation | LCAR-B38M/JNJ-4528 - |
| antibody conjugated with | half life extended form of | compound dual epitope targeting |
| mertansine via a | AMG420 targeting the BCMA, | CAR-T cells that binds two |
| noncleavable linker | which induces specific and | BCMA epitopes |
| | effective T -cell dependent | |
| | cytotoxicity against MM cells | |
| | [Id., citing Goldstein, RL et al. | |
| | Blood Adv. (2020) 4: 4180-94] | |
| MEDI2228: fully human | CC-93269 (formerly EM801) - | Orvacabtagene Autoleucel - |
| anti-BCMA that provides a | bispecific antibody binding | BCMA-directed CAR-T cell with |
| pyrrolobenzodiazepine | with an anti-BCMA to BCMA | a fully human binder with in vitro |
| (PBD) dimer conjugation via | and CD3 T cells with extended | activity, even in patients with low |
| a protease-cleavable linker | half life; asymmetric two arm | BCMA cells [Id., citing |
| | IgG1-based humanized Ab | Mailankody, S. et al. Blood |
| | binding bivalent to BCMA and | (2018) 132: 957] |
| | monovalent to CD3ε | |
| HDP-101 - anti-BCMA- | REGN5458 and 5459: fully | CT103A - second generation |
| targeted amanitin conjugates | humanized antibody binding to | CAR incorporating a fully human |
| | BCMA on MM cells and to the | ScFv sequence |
| | CD3 on immune cells, inducing | |
| | T cell death [Id., citing Cooper, | |
| | D. et al. Blood (2019) 134: | |
| | 3176] | |
| | PF-06863135 - fully | Decartes-08: RNA generated |
| | humanized IgG CD3 bispecific | anti-BCMA CD8 CAR-T cell |
| | monoclonal antibody that | through production of cytokines |
| | utilizes anti-BCMA and anti- | such as IFNμ, TNFα and IL-2 |
| | CD3 targeting arms paired by | [Id., citing Lin, L. et al. |
| | hinge mutation technology | Leukemia (2021) 35: 752-63] |
| | placed in an IgG2A backbone | |
| | [Id., citing Demel, I. et al. Br. J. | |
| | Haematol. (2021) 193: 705-22; | |
| | Panowski, SH et al. Blood | |
| | (2016) 128: 383] | |
| | Teclistamab (JNJ) is a duobody | Allogeneic BCMA CAR-T cells |
| | characterized as a humanized | An alternative therapeutic approach |
| | IgG4 bispecific antibody | offers BCMA 1-R2 CAR-T cells, |
| | binding to both BCMA and | which are anti-BCMA allogeneic |
| | CD3 receptors of T cells | CAR-T cells from healthy donors |
| | | [Id., citing Sommer, C. et al. Mol. |
| | | Ther. (2019) 27: 1126-38]. To |
| | | reduce the risk of graft versus host |
| | | disease (GvHD), TRAC gene was |
| | | replaced by transcription activator- |
| | | like effector nucleases (TALEN). |
| | | Moreover, CD52 was additionally |
| | | knocked out by TALEN to make |
| | | BCMA 1-R2 CAR-T cells resistant |
| | | to anti-CD52 antibodies (e.g., |
| | | alemtuzumab). The purpose of |
| | | manufacturing CD52-deficient T |
| | | cells is to allow the application of |
| | | alemtuzumab concurrently or prior |
| | | to engineered T cells, which |
| | | potentially eliminate CD52 |
| | | expressing host T cells, to mediate |
| | | engraftment and avoid allorejection |
| | | [Id., citing Depil, S. et al. Nat. Rev. |
| | | Drug Discov. (2020) 19: 185-99; |
| | | Poirot, L. et al. Cancer Res. (2015) |
| | | 75: 3853-64]. Moreover, the safety |
| | | of BCMA 1-R2 CAR-T cells is |
| | | enhanced by the incorporation of an |
| | | intra-CAR rituximab binding |
| | | domain as an off switch, enabling |
| | | the elimination of CAR-T with |
| | | rituximab [Id., citing Fang, Y. Hou, |
| | | J. Mil. Med. Res. (2021) 8: 9]. |
| | TNB-8383B: one arm with H | |
| | and L chain with α-CD3 and a | |
| | second arm only with H chain, | |
| | binding dually with high- | |
| | affinity to BCA domains. | |
| | αCD3 binds and activates | |
| | CD4/CD8 T cells | |
| | preferentially; thus release of | |
| | cytokines could be reduced | |
| | [Id., citing Buelow, B. et al. J. | |
| | Clin. Oncol. (2018) 36: 8034] | |

### BCMA as a Therapeutic Strategy for AML

There is little if any data connecting BCMA as a therapeutic strategy to AML or myeloid cells.

No AML reference is included in a systemic review of the scientific literature reporting on patients of any age with hematologic malignances assessing BCMA expression (protein or mRNA) in hematologic malignances, from which animal studies, studies using nonhuman and/or non hematologic cell lines, studies investigating non-hematologic conditions and review articles were excluded. Khattar [Khattar, P. et al. Blood (2017) 130 (Suppl. 1): 2755] reported that BCMA expression in normal lymphoid tissue is restricted to plasma cells and germinal center B cells. BCMA expression was not detected in other tissues (epithelium, T cells, dendritic cells, and histocytes/macrophages) using a clinical grade assay. BCMA shows a high level of expression in B cell lineage malignancies and plasma cell neoplasms, but T cell lymphoma, Hodgkin lymphoma, and myeloid and lymphoblastic lymphoma/leukemia were negative for BCMA immunostain. [Id.]

While BCMA deficiency has been reported to lead to impaired survival of long-lived marrow plasma cells [Bolkun, L. et al. J. Cancer (2016) 7:1979-83, citing O'Connor, B P et al. J. Exp. Med. (2004) 199 (1): 91-8], a soluble form of BCMA was reported to inhibit the proliferative activity of APRIL in vitro and to decrease proliferation of HT29 colon carcinoma cells in nude mice. [Id., citing Rennert, P. et al. J. Exp. Med. (2000) 192 (11): 1677-84]. Additionally, in xenograft models of lung and colon cancer, intratumoral delivery of soluble BCMA significantly reduced tumor growth in mice that had been treated with the soluble receptor. Id., citing Rennert, P. et al. J. Exp. Med. (2000) 192 (11): 1677-84

Although the anti-apoptotic activity of APRIL and BAFF has been demonstrated in B-lymphoma, MM, and chronic lymphocytic leukemia (B-CLL) [Bolkun, L. et al. J. Cancer (2016) 7:1979-83, citing Chiu, A. et al. Blood (2007) 109 (2): 729-39; Kern, C. et al. Blood (2004) 103 (2): 679-88; Moreaux, J. et al. Blood (2004) 103 (8): 3148-57], only limited studies have reported on the role of APRIL and BAFF in regulating apoptotic processes in AML.

To determine whether baseline quantification of peripheral blood CD33+ blast cells expressing BAFF, APRIL and their receptors could improve prognosis of treatment response, a study of 24 patients with newly diagnosed AML, median age 55 years, range 21-65 was performed by Bolkun et al and reported in 2016 [Bolkun, L. et al. J. Cancer (2016) 7:1979-83]. All patients had a normal karyotype and none suffered from mutated CEBPA, NPMI or FLT3-ITD. AML patients were treated with a 7 day induction chemotherapy regimen [Id., citing Vardiman, JW. Et al. Blood (2009) 114 (5): 937-51]; after induction, the morphological response was evaluated: 12 patients achieved complete remission after first induction and 12 were nonresponders.

Among groups of patients with varying clinical responses to applied chemotherapy, significant differences were found in frequencies of CD33+ blasts expressing APRIL and BAFF, but not their mean fluorescence intensity (MFI). Nonresponders (NR) showed elevated baseline frequencies of APRIL and BAFF-expressing CD33+ blasts, compared to patients who achieved complete remission (CR). There was no difference in plasma levels of APRIL and BAFF between CR and NF patients.

With regard to APRIL and BAFF receptors, AML patients who did not respond to induction chemotherapy presented barely detectable frequencies of CD33+BCMA+ cells (below 1%). In CR patients, substantially elevated but still small frequencies of those cells were found. No differences in the frequencies of AML blasts expressing TACI and BAFF-R were found. Patients who further developed CR to induction chemotherapy presented with significantly elevated MFI of BAFF-R and TACI, but not BCMA on AML cells when compared to NR.

PCR analysis of expression levels of APRIL. BAFF, BCMA, BAFF-R and TACI showed no differences in expression level of BAFF, BAFF-R and TACI in CD33+AML cells (p>0.05). A tendency was observed at a threshold of statistical significance toward higher expression of APRIL in NR when compared to CR patients. CR patients showed higher BCMA expression when compared to NR.

Because several studies indicated that aberrant DNA methylation could play a role in the progression of numerous neoplasms, including AML [Id., citing Toyota, M. et al. Blood (2001) 97 (9): 2823-9], expression of membrane BCMA on CD33+AMI blasts was confirmed by immunofluorescence staining. Significant negative correlation between the level of methylation of the BCMA promoter and the frequencies of CD33+BCMA+ cells was observed, suggesting possible contribution of methylation of BCMA to the resistance of AML cells to the therapy. No significant relationship was observed between BCMA methylation status and clinical features that may influence gene methylation (e.g., age, smoking). At baseline, BCMA expression on AML cells could be detected only in CR but not NR patients. Significant negative correlation was observed between the expression of BAFF, but not APRIL, and BCMA in AML cells suggesting a possible regulatory function of BAFF expression.

Next DNA methylation status of promoter sequences of the BCMA gene were analyzed. No differences in methylation levels with regard to the type of clinical response to the therapy was observed; significant negative correlation was found between the level of methylation of the BCMA promoter and the frequencies of CD33+BCMA+ cells.

Finally, AML cells were treated with cytosine arabinoside (araC) either in the presence or absence of recombinant human APRIL and BAFF in order to directly evaluate the pro-survival effect of APRIL and BAFF on CD33+ cells. Ara C as expected significantly decreased frequencies of live CD33+AML blasts. Stimulation with APRIL significantly reduced AraC-related induction of cell death in NR but not in CR patients. A trend (at a threshold of statistical significance) was observed to increase frequencies of viable cells after BAFF stimulation in the presence of Ara C. These results were interpreted to confirm observations suggesting a possible involvement of BCMA in regulating apoptosis of AML cells and the usefulness of BCMA as a potential marker to predict treatment response to induction chemotherapy. However, the exact function of BCMA and regulation of its expression in AML blasts remains unknown.

Experiments by Matthes, et al. concerned plasma cell development and differentiation in myeloid precursor cells from human bone marrow. [Matthes, T. et al. Blood 2011) 118 (7): 1838-44]. Matthes teaches that it was known that after their generation in lymphoid organs, antibody-producing plasma cells (PCs), highly dependent on environmental factors for survival ([Id., citing Sze, D M et al. J. Exp. Med. (2000) 192 (6): 813-21], enter the bloodstream to seek survival niches, such as the niches present in the bone marrow. [Id., citing Slifka, M K et al. J. Virol. (1995) 69 (3): 1895-1902; Manz, R. A. et al. Nature (1997) 388 (6638): 133-343] The chemokine CXCL-12 mediates BM homing of PCs. [Id., citing Tokoyoda, K. et al. Immunity (2004) 20 (6): 707-18] Once in the BM, PCs are held in a VLA-4/VCAM-1 adhesion manner in specific survival niches. [Id., citing Tokoyoda, K. et al. Immunity (2004) 20 (6): 707-18]. These niches are believed to provide all the PC survival factors needed. It is widely accepted that these niches are limited in number, because BM aspiration revealed that PCs constituted no more than 0.5% to 1% of the total BM cellularity.

APRIL has been implicated in PC survival [Id., citing Mackay, F. et al. Annu. Rev. Immunol (2003) 21:231-64] Recombinant APRIL was reported to ameliorate PC survival in vitro [Id., citing O'Connor, B P et al. J. Exp. Med. (2004) 199 (10:91-98; Huard, B. et al. J. Clin. Invest. (2008) 118 (8): 2887-95; Bossen, C. et al. Blood (2008) 111 (3): 1004-12; Belnoue, E. et al. Blood (2008) 111 (5): 2755-64]. This function was confirmed in vivo in APRILnull mice [Id., citing Belnouc, E. et al. Blood (2008) 111 (5): 2755-64; Castigli, E. et al. Proc. Natl Acad. Sci. USA (2004) 101 (11): 3903-8; Benson, M J et al. J. Immunol. (2008) 180 (6): 3655-9]. APRIL binds to two receptors, BCMA and TACI [Id., citing Kalled, S L et al. Curr Dir. Autoimmun. (2005) 8:206-42]. In addition, APRIL uses heparin sulfate proteoglycans (HSPGs as coreceptors [Id., citing Ingold, K. et al. J. Exp. Med. (2005) 201 (9): 1375-83; Hendriks, J. et al. Cell Death Differ. (2005) 12 (6): 637-48]. The coexpression of B-cell maturation antigen and the heparin sulfate proteoglycan (SSPG) CD138 by PCs [Id., citing O'Connor, B P et al. J. Exp Med. (2004) 199 (1): 91-98; Wijdenes, J. et al. Br. J. Hacmatol. (1996) 94 (2): 318-23] is consistent with the induction of a survival signal by APRIL in these terminally differentiated nonproliferative cells.

In situ studies showed that megakaryocytes [Id., citing Winter, O. et al. Blood (2010) 116 (11): 1867-75], cosinophils and a subset of monocytes [Id., citing Chu, V T et al. Nat. Immunol. (2011) 12 (2): 151-9] produce APRIL in mouse BM. In addition, in vitro-differentiated osteoclasts also produce APRIL [Id., citing Bond, D. et al. Blood (2004) 104 (1): 3169-72; Moreaux, J. et al. Blood (2005) 106 (3): 1021-30; Yacoby, S. et al. Leukemia (2008) 22 (2): 406-13]. All these cell types constitute only a minor fraction of total BM cells.

Matthes et al studied human BM and observed production of APRIL mediated by cells from the myeloid lineage, the most abundant BM compartment, both at their immature and mature state using two antibodies: a Stalk-1 antibody to reveal cells producing APRIL and April-2, an antibody detecting the secreted part of APRIL. They elegantly demonstrated that APRIL secretion by myeloid precursor cells in BM plasma serves to sustain PC survival.
(1) Myeloid precursor cells produce high amounts of APRIL before getting fully mature and reaching the circulation, but the surrounding tissue does not retain secreted APRIL. Up to 50% human BM cells were positive for APRIL, but staining with Aprily-2 was negative. APRIL-producing cells readily secrete all the APRIL they produce. The BM microenvironment around these cells does not retain secreted APRIL, probably because of a lack of HSPG expression.
(2) APRIL production by granulocytes starts early in their differentiation, at the promyelocyte stage; the production peaks at the myelocyte stage and then drops with continuing maturation. Immature and mature cells are both equipped to secrete the APRIL that they produce.
(3) there was no close-contact between APRIL-producing myeloid cells and PCs in human BM indicating that APRIL producing cells are not part of the PC survival niche.
(4) Experiments reproducing myelopoiesis in vitro by incubating HSCs with the early hematopoietic factors SCF, TPO, FLT-3L acting on noncommitted cells that can induce multiple lineage pathways, and G-CSF selectively driving differentiation toward the granulocyte lineage [Id., citing Barreda, D R et al. Dev. Comp. Immunol. (2004) 28 (5): 509-54] showed that cytokines acting early in hematopoiesis are involved in APRIL production by myeloid precursor cells.
(5) Experiments to analyze the role of APRIL for BM PC survival in mice using APRIL blockade with mouse antagonist mAb Apry-1-1 showed that a one-shot treatment with the antagonist 1 day before cell transfer was sufficient to reduce the number of recovered PCs by more than 2-fold. In mouse BM, immature myeloid cells are present in a cell population defined by low expression of the myeloid marker Gr-1 [Id., citing Ueda, Y. et al. J. Exp. Med. (2005) 201 (11): 1771-80]. The experiments showed that Gr-1^{lo} BM cells contained early stages of myeloid differentiation (myeloblasts, promyelocyte, and myelocytes) characterized by the presence of large cells with a small nuclear opening when present. Eosinophils, reported to produce APRIL in mouse BM, were not predominant in this population. Gr-1^{hi} cells contain more mature cells (metamyelocytes, band cells, and neutrophils) characterized by the presence of cells harboring a nucleus with a larger opening that progresses toward full segmentation. Q-PCR on FACS-sorted purified cells showed that immature Gr 11º cells expressed more APRIL mRNA than mature Gr-1^{hi} cells. In coculture experiments, immature Gr-1^{lo} cells were more efficient than mature Gr-1^{hi} cells in sustaining PC survival; this enhanced PC survival was APRIL dependent.

The present disclosure provides BCMA as a new target for post-consolidation immunotherapy for eligible patient populations having AML with high-risk disease. The immunotherapy includes CAR-T for binding and then killing through receptor mediated killing; bispecific T cell engagers whose binding gets T cells close to the target cell, BCMA inhibitors which signal through NFκB suppression even without any engagement of immune cells; antibody-drug conjugates, and neutralizing antibodies. Data is presented showing that BCMA is present on the cell surface of AML cell lines and patient samples of young adults aged 26-45 and demonstrating in vitro efficacy of BCMA targeting bispecific T cell engager on AML.

### SUMMARY OF THE INVENTION

The present disclosure provides a method for treating an eligible subject with acute myeloid leukemia (AML) including high risk disease features comprising administering to the subject post-consolidation a targeted immunotherapy comprising an immunotherapeutic agent that specifically targets B cell maturation antigen. (BCMA).

According to some embodiments of the method, the eligible subject with AML is a child; or the eligible subject with AML is a child less than 2; or the eligible subject with AML is a teenage child aged 13-19, inclusive; or the eligible subject with AML is an adult; or the eligible subject with AML is an adult less than 60; or the eligible subject with AML is a young adult, aged 26-50, inclusive, or the eligible subject with AML is an adult aged 60-69, inclusive; or the eligible subject with AML is 70 or older.

According to some embodiments of the method, the high risk disease features include biologic features, clinical features, or both.

According to some embodiments, the high risk biological features include antecedent hematological disorders, presence of ≥20% BM blasts in the bone marrow, 0, 1, 2, 3 or more clonal cytogenetic abnormalities, and molecular abnormalities.

According to some embodiments, the antecedent hematological disorder is myelodysplastic syndrome, refractory AML, AML in remission, or mixed-phenotype acute leukemia.

According to some embodiments, the clinical features include comorbidities, measurable residual disease at time of complete remission, refractory to induction chemotherapy; remission after induction therapy, relapsed subject in remission, AML arising out of an antecedent hematologic disorder; and AML in the elderly.

According to some embodiments, the high risk molecular feature is an FLT3 mutation, an NPM1 mutation; an isocitrate dehydrogenase 1 or 2 (IDH1/2) mutation, a RUNX1 mutation, a DNMT3A mutation, a TET2 mutation, a TP53 mutation, or a combination thereof.

According to some embodiments, the FLT3 mutation is an FLT3-ILD mutation.

According to some embodiments, the high risk cytogenetic features include a chromosomal translocation and a monosomy of a somatic chromosome. According to some embodiments the translocation is t (8;21) (q22;q22.1); RUNX1-RUNXIT1);

According to some embodiments, high risk features in the elderly include one or more of co-morbidities, a high incidence of cytogenetic abnormalities involving monosomies 5 and 7 and chromosome 17 abnormalities, a high incidence of multiple mutations including TP53, and a high incidence of secondary/therapy-related AML. According to some embodiments, wherein the comorbidities include one or more of hypertension; diabetes; organ dysfunctions including cardiac, pulmonary and renal abnormalities).

According to some embodiments of the method, the immunotherapy includes an immune cell engager” (“ICE”) selected from a T cell engager, a natural killer (NK) cell engager and cytotoxic/phagocytic cell engagers; and/or an immune checkpoint therapy comprising an immune checkpoint inhibitor, and/or a gamma-secretase inhibitor.

According to some embodiments, the immune cell engager is a bispecific T cell engager (BiTe) comprising a BCMA-targeting molecule connected through a peptide linker (ranging from 3 to 20 amino acids in length, inclusive, i. c., 3. 4, 5, 6, 7, 8, 9, 10, 11, 12, 13, 14, 15, 16, 17, 18, 19, or 20 amino acids) to a CD3 targeting molecule, wherein the CD3 targeting molecule activates a specific chain of the CD3 complex associated with the T cell receptor (TCR) complex and participates in assembly of an immunological synapse mediating BCMA-specific cytotoxicity; or the immune cell engager is a CD16 or an NKG2D receptor-directed bispecific NK cell engager that activates NK cells targeting BCMA-specific cytotoxicity; or the immune engager is a chemically linked bispecific molecule engaging the non-ligand binding site of the high-affinity receptor for immunoglobulin G (FcγRI, also known as CD64) selectively expressed by a population of cytotoxic/phagocytic immune cells targeting BCMA antibody-dependent cell-mediated cytotoxicity of AML blast cells.

According to some embodiments, the BCMA-targeting molecule is a BMCA targeting scFv fragment and the CD3-targeting molecule is an scFv fragment.

According to some embodiments, the CD64 expressing population of cytotoxic/phagocytic immune cells is a population of monocytes, macrophages, dendritic cells or cytokine-activated neutrophils.

According to some embodiments, the T cell immune engager is a BiTE that targets CD3 on T cells and targets BCMA expressed on relapsed/refractory AML blasts.

According to some embodiments, the BiTE is teclistamab, elranatamab, AMG-701, AMG 420 (formerly BI 836909), REGN5458, or TNB-383B.

According to some embodiments, the checkpoint inhibitor is lambrolizumab/pembrolizumab (KEYTRUDA®), nivolumab (OPDIVO®), atezolizumab, TECENTRIQ®, or ipilimumab (YERVOY®).

According to some embodiments, wherein the gamma-secretase inhibitor is LY3039478/JSMD194, dihydroergocristine (DHEC), RO4929097; LY900009; MK-0752; PF-03084014; BMS-986115; GSI-136; AL-101; or Nirogacestat.

According to some embodiments, the immunotherapy includes a BCMA-targeted antibody drug conjugate or a BCMA-targeted CAR-T cell therapy described in Table 1.

### BRIEF DESCRIPTION OF THE DRAWINGS

FIG. 1 schematically depicts the role of CD4+ T cells in modulating immune responses against cancer. As shown in FIG. 1, CD4+T cells play a central role in regulating the host immune responses through the following mechanisms. (a) CD4+T cells provide crucial help in the priming of CD8+T cells via activation of antigen presenting cells (APCs). (b) CD4+T cells secrete cytokines required for maintaining CD8+T-cell function and proliferation. (c) CD4+T cells can inhibit tumor growth directly or indirectly. (d) CD4+T cells provide help for B-cell activation. Thus, both CD8+ T cells and antibody production require the crucial help of CD4+T cells. Taken from Wang, R-F. Trends in Immunol. (2001) 22 (50): 269-76.

FIG. 2 schematically depicts a reconciled model for hematopoietic stem cell differentiation HSCs first differentiate into MPP1/short-term HSCs (ST-HSC), then give rise to MPP2, MPP3 and MPP4 lymphoid primed MPPs (LMPP). MPP2 can generate premegakaryocye/erythroid progenitor (pre MegE), and subsequently, pre MegE gives rise to platelets through megakaryocyte progenitors (MkPs) or produce erythrocytes through Pre CFU-E. MPP3 mostly give rise to granulocyte and monocyte lineages, and MPP4 (LMPP) mainly contribute to lymphocytes. Taken from Cheng, H. et al. New paradigms on hematopoietic stem cell differentiation. Protein Cell (2020) 11 (1): 34-44].

FIG. 3 schematically shows immune escape strategies employed by AML blasts. AML blasts can hamper T- and NK-cell effector functions by aberrantly overexpressing inhibitory T-cell ligands (i.e. PD-L1, Gal-9, CD155, CD112, CD86) (1), or by releasing soluble forms of NKG2DL AML blasts promote T-cell exhaustion and apoptosis, drive the expansion of regulatory T cells (Tregs) and myeloid-derived suppressor cells (MDSCs), and induce the switch of macrophages to tumor-associated macrophages (TAMs) (3) by altering the cytokine milieu and through the release within the BM niche of other soluble factors such as reactive oxygen species (ROS), indoleamine 2,3-dioxygenase-1 (IDO1), arginase II (ArgII), and extracellular vesicles (EVs) (4). Moreover, AML blasts reduce their expression of antigen presentation molecules, thus hiding themselves from immune cells such as dendritic cells (DCs) and macrophages (5). Taken from Tettamanti, S. et al. Leukemia (2022) 36 (1): 13-22.

FIG. 4 schematically depicts the signaling pathway for B cell maturation antigen (BCMA) in multiple myeloma. BCMA has two agonist ligands: a proliferation-inducing ligand (APRIL) and B cell activating factor (BAFF), which are mainly secreted by the bone marrow (BM) stromal cells, osteoclasts, and macrophages in a paracrine manner in the BM. APRIL exhibits a much higher binding affinity to BCMA than BAFF, and it also binds to TACI, while BAFF endorses more selectivity to BAFF-R. Multiple growth and survival signaling cascades are subsequently activated in multiple myeloma (MM) cells, most frequently through NF-κB, leading to upregulation of anti-apoptotic proteins and production of cell adhesion molecules, angiogenesis factors, and immunosuppressive molecules. These lead to increased survival of MM cells. Membrane BCMA can be cleaved by γ-secretase and released to the plasma as soluble BCMA (sBCMA). sBCMA can bind to APRIL and BAFF, which may interfere with the activation of BCMA signaling pathways. Taken from Yu et al. BCMA-targeted immunotherapy for multiple myeloma. J. Hematol. & Oncology. (2020) 13: article 126. Doi.org/10.1186/s13045-020-00962-7.

FIG. 5 diagrammatically illustrates the pathways to antigen presentation [taken from Yewdell, J W and Dolan, BP. Nature (2011) 471 (7340): 581-82].

FIG. 6 schematically depicts the mechanism of action of immune cells engagers. Immune cell engagers are able to redirect immune effector cells and create an artificial immune synapse between tumor cells (targeting a tumor-associated antigen) and T cells (engaging CD3 or costimulatory molecules, such as CD28 or 4-1BB), NK cells (engaging CD16 or NKG2D) and cytotoxic/phagocytic cells (engaging CD64). [Taken from Fuca, G. et al. ESMO (2021) 6 (1): doi.org/10.1016/j.esmoop.2020.10046].

FIG. 7A, FIG. 7B, FIG. 7C, FIG. 7D, FIG. 7E and FIG. 7F provides mean fluorescence intensity data showing that BCMA is highly expressed in AML cell lines and patient cells. FIG. 7A. Three human MM cell lines (MM IS, U266, NCI-H929 were analyzed for the expression of the MM-associated antigen BCMA and the AML-associated antigens CD33, Flt3, and CD123. Mean and SEM of median fluorescence intensities (MFI) are presented.

FIG. 7B. Nine human AML cell lines (THP-1, OCI-AML-3, MOLM-13, K052, KASUMI-1, SET-2, NKM-1, HL-60, HEL) were analysed for expression of the MM-associated antigen BCMA and the AML-associated antigens CD33, Flt3, and CD123. Mean and SEM of MFI are presented. FIG. 7C. Healthy human peripheral blood mononuclear cells (PBMCs) and bone marrow mononuclear cells (BMMC) were analysed for the expression of BCMA, Flt3, and CD123. Mean and SEM of MFI are presented. FIG. 7D. AML patient cells were analyzed for the expression of BCMA, Flt3, and CD123. FIG. 7E. Healthy hematopoietic cells (CD34+HSPC, PBMC, BMMC, white columns) as well as AML cell lines (gray columns) and AML patient cells (black column) were analyzed for the expression of BCMA. Mean and SEM of MFI are presented. FIG. 7F. Summary of BCMA positivity (frequency of BCMA positive cells) of AML cells from 33 patients. ND: newly diagnosed; R: relapsed.

FIG. 8A, FIG. 8B, and FIG. 8C show that while U266 cells were more sensitive to BCMA BiTE mediated killing than AML cells, BCMA BiTE treatment mediates killing of AML cells in vitro.

FIG. 8A is a plot of percent killing vs. time for U266-luc human multiple myeloma cells (left) and MOLM-13-luc human AML cells (right) cultured in the presence of human peripheral blood mononuclear cells (PBMC) at an effector: target ratio of 20:1. BCMA×CD3 BITE (10 or 50 nM) was added to the media and relative luminescence units (RLU) were measured after 24 and 48 h. Controls included the following conditions: untreated target cells, target cells+PBMC, target cells+BiTE. Normalized values (percentage of untreated target cells) are presented (Mean and SEM).

FIG. 8B is a plot of percent killing vs. time for U266-luc human multiple myeloma cells (left) and OCI-AML3-luc human AML cells (right) cultured in the presence of human PBMC at effector: target ratios of 10:1 and 20:1. BCMA×CD3 BITE (0.4 nM) was added to the media and RLU were measured after 24, 48 and 72 h. Controls included the following conditions: untreated target cells, target cells+PBMC, target cells+BiTE. Normalized values (percentage of untreated target cells) are presented (Mean and SEM).

FIG. 8C is a plot of percent killing of U266-luc cells (left), MOLM-13-luc cells (middle) and OCI-AML3 cells (right) cultured in the presence of human PBMC at effector: target ratios of 10:1 (U266), 20:1 (MOLM-13) and 30:1 (OCI-AML3). BCMA×CD3 BiTE was added to the media at the indicated concentrations and RLU were measured after 24, 48 and 72 h. Controls included the following conditions: untreated target cells and target cells+PBMC. Normalized values (percentage of untreated target cells) are presented (Mean and SEM).

FIG. 9 shows that the BCMA gene (TNFRSF17) is expressed by all AML subsets. Median normalized expression (transcripts per million of BCMA by AML cells cells (transcripts permillion) based on the French-American-British (FAB) classification. The figure was created with the University of Alabama at Birmingham Cancer data analysis Portal (UALCAN) platform using TCGA bulk RNA sequencing gene expression data sets.

FIG. 10 provides a Kaplan Meyer analysis of AML probability of survival comparing patients with high (top 10%) and low (bottom 10%) BCMA gene normalized expression levels (transcripts per million, TPM). The difference between the groups was analyzed by Log-rank test. The figure was created with the OncoInc platform using The Cancer Genome Atlast program (TCGA) survival data. It shows that shows that high BCMA expression levels are associated with a poor prognosis in AML patients.

FIG. 11A, FIG. 11B and FIG. 11C shows that BCMAxCD3 T cell engager (TCE) treatment mediates killing of AML cells in vitro. FIG. 11A: OCI-AML3-luc human AML cells were cultured in the presence of human PBMC at an effector: target ratio of 20:1. BCMA×CD3 TCE (0.4 nM) was added to the media and relative luminescence units (RLU) were measured after 24, 48 and 72 h. Controls included the following conditions: untreated target cells, target cells+PBMC, target cells+TCE. Normalized values (killing percentage based on untreated target cells) are presented (Mean and SEM). FIG. 11B: THP-1-luc human AML cells were cultured in the presence of human PBMC at an effector: target ratio of 10:1. BCMA×CD3 TCE (10 nM) was added to the media and RLU were measured after 24, 48 and 72 h. Controls included the following conditions: untreated target cells, target cells+PBMC, target cells+TCE. Normalized values (killing percentage based on untreated target cells) are presented (Mean and SEM). FIG. 11C: MOLM-13-luc human AML cells were cultured in the presence of human PBMC at an effector: target ratio of 20:1. BCMA×CD3 TCE (10 or 50 nM) was added to the media and relative luminescence units (RLU) were measured after 24 and 48 h. Controls included the following conditions: untreated target cells, target cells+PBMC, target cells+TCE. Normalized values (killing percentage based on untreated target cells) are presented (Mean and SEM).

FIG. 12A and FIG. 12B shows that BCMAxCD3 TCE treatment displays strong antileukemic activity in an AML xenograft model. FIG. 12A. NSG mice received 2.5×106 luciferase-expressing THP-1 cells intravenously. After 10 days engraftment was confirmed, and mice were assigned to the following groups: 1. Placebo (PBS) I.V. on day 0; 2. Human CD8 T cells (1×106) I.V. on day 0; 3. Human CD8 T cells (1×106) I.V. on day 0 followed by BCMAxCD3 bispecific T cell engager (TCE) 20 mg/kg I.V. on days 1, 4, 8, 11, 15, 18, 22; 4. Human CD8 T cells (1×106) I.V. on day 0 followed by TCE 80 mg/kg I.V. on days 1, 4, 8, 11, 15, 18, 22. A: In vivo bioluminescence imaging between days 4 and 29 of treatment. Mean and SEM are presented (n=3-4). FIG. 12B: Kaplan Meier analysis of survival.

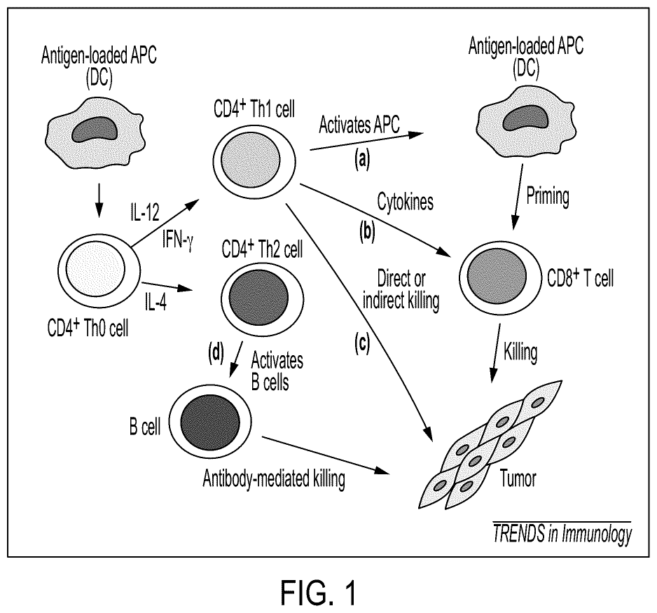

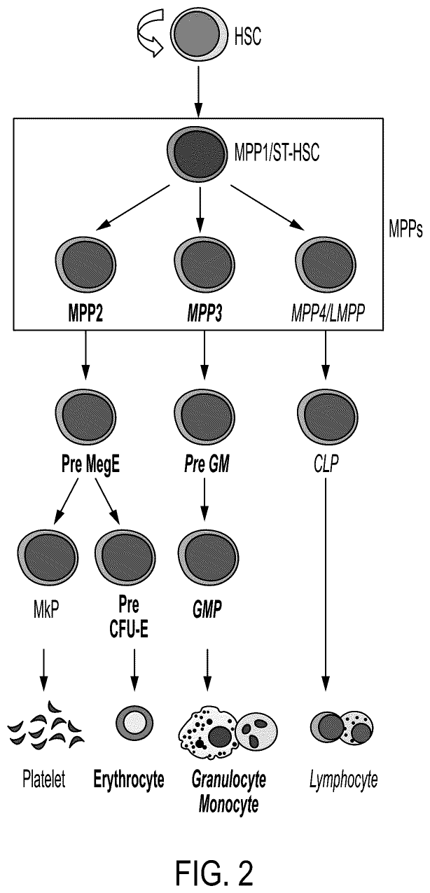

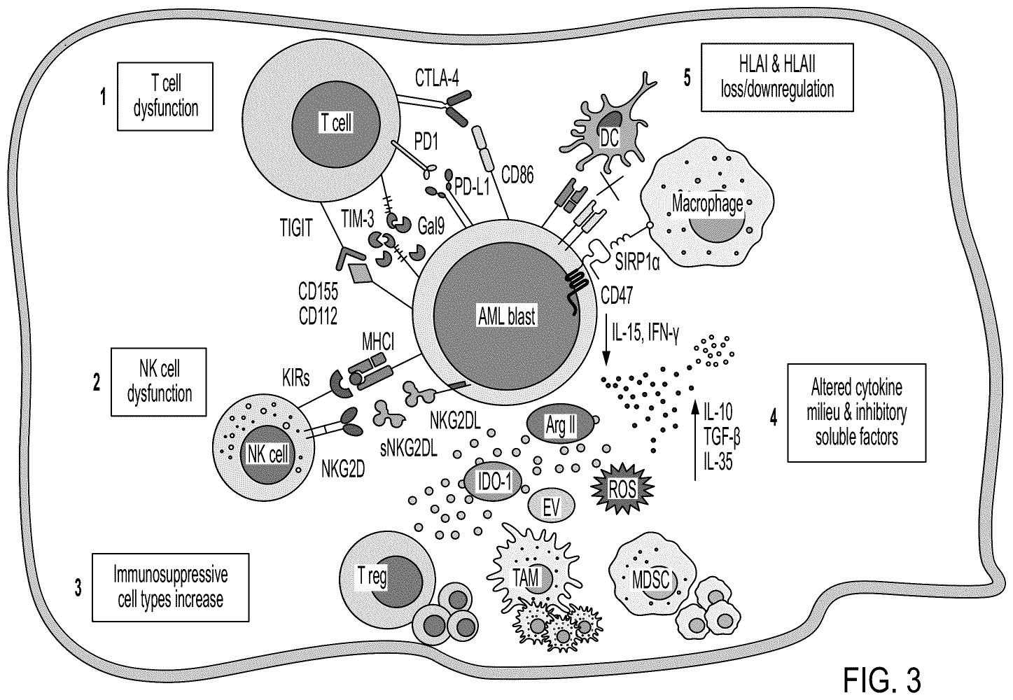

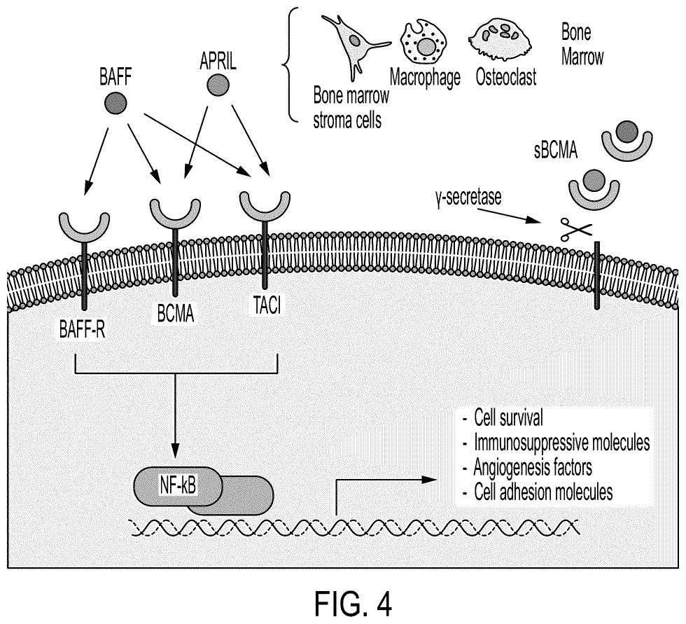

### DETAILED DESCRIPTION OF THE INVENTION

### Glossary

The term “4-1BB (CD137, tumor necrosis factor receptor superfamily 9) is an inducible costimulatory receptor expressed on activated T and natural killer (NK) cells. 4-1BB ligation on T cells triggers a signaling cascade that results in upregulation of antiapoptotic molecules, cytokine secretion, and enhanced effector function. On NK cells, 4-1BB signaling can increase antibody-dependent cell-mediated cytotoxicity.

The terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

The term “administering” and its various grammatical forms as it applies to a mammal, cell, tissue, organ, or biological fluid, as used herein is meant to refer without limitation to contact of an exogenous ligand, reagent, placebo, small molecule, pharmaceutical agent, therapeutic agent, diagnostic agent, or composition to the subject, cell, tissue, organ, or biological fluid, and the like. It includes in vivo administration, as well as administration directly to tissue ex vivo. Generally, compositions may be administered systemically either orally, buccally, parenterally, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally. “Administration” can refer, e.g., to therapeutic, pharmacokinetic, diagnostic, research, placebo, and experimental methods. “Administration” also encompasses in vitro and ex vivo treatments, e.g., of a cell by a reagent, diagnostic, binding composition, or by another cell. It should be understood that “administration” includes co-formulation (meaning formulated together) as well as administration via one or more pharmaceutical compositions administered concurrently (meaning at the same time, including, e.g., co-administration) or sequentially (meaning coming after in time or order). For cell therapy, administration can be intravenously (iv), e.g., by infusion.

The term “adaptor protein” as used herein refers to a protein that contains a series of protein-binding sites that link respective interaction partners to each other and facilitate the generation of larger signaling complexes.

The term “adoptive immunotherapy” also known as “cellular immunotherapy” is a type of immunotherapy in which T cells are given to a patient to help the body fight diseases. Types of adoptive cell therapy include chimeric antigen receptor T-cell (CAR T-cell) therapy and tumor-infiltrating lymphocyte (TIL) therapy.

The term “adverse event” or “AE” as used herein refers to an unfavorable medical event that occurs in a subject who is given a pharmaceutical product, but does not necessarily have a causal relationship with the treatment. It may be any adverse and unwanted signs (including an abnormal laboratory result), symptoms, or temporary illness associated with the use of the product, whether or not it is related to the product. The correlation between adverse events and test medications is: affirmative, likely related, may be relevant, may be irrelevant, and certainly not relevant. The severity of adverse events according to the National Cancer Institute's Common Terminology Criteria for Adverse Events (CTCAE) Scale is shown in Table 2 below.

TABLE 2
Severity of Adverse Events

| CTCAE Scale | Description |
|---|---|
| Mild (Grade 1) | Asymptomatic or mild; or only seen clinically or |
| | diagnostically; or no treatment required |
| Moderate (Grade 2) | Requires minor, local or non-invasive treatment; |
| | age-appropriate instrumental activities of daily |
| | life are limited |
| Severe (Grade 3) | Serious or medically significant but not immediately |
| | life-threatening; resulting in hospitalization or |
| | prolonged hospital stay; disability; limited daily |
| | activities of the individual |
| Life Threatening | Life-threatening; urgent treatment required |
| (Grade 4) | |
| Death (Grade 5) | Death associated with AE |

The term “anergy” as used herein refers to a state of lymphocyte nonresponsiveness to specific antigen induced by an encounter of the lymphocyte with cognate antigen under less than optimal conditions, such as in the absence of costimulation.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins the molecules of which possess small areas of their surface that are complementary to small chemical groupings on their targets. These complementary regions (referred to as the antibody combining sites or antigen binding sites) of which there are at least two per whole antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, K and 2; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (VK and VA) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human V_{I} chains with the heavy chain variable region (V_{H}) domain of a mouse monoclonal antibody reactive with an antigen of interest; (ii) selecting half-human Fabs on that antigen (iii) using the selected V_{I} genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1 antibody molecule in the mouse myeloma.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques.

An antibody may be from any species. The term antibody also includes binding fragments of the antibodies of the invention. Binding fragments of an antibody can be produced by recombinant DNA techniques, or by enzymatic or chemical cleavage of intact antibodies. Exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. Sec, for example, Bowie et al. Science 253:164 (1991), which is incorporated by reference in its entirety. An antibody other than a “bispecific” or “bifunctional” antibody is understood to have each of its binding sites identical.

Bispecific antibody (BsAb) design formats. Generally, bispecific antibodies can be divided into two major classes: those bearing an Fc region and those lacking an Fc region, which are normally smaller than the IgG and IgG-like bispecific molecules comprising an Fc. The presence of an Fc region has consequences for Fc mediated effector functions, such as antibody-dependent cellular cytotoxicity (ADCC), complement fixation (CDC), and the long half-life resulting from their larger size and Fc-Rn-mediated recycling processes. [Kontermann, RE and Brinkmann (U., Drug Discovery Today (2015) 20 (7): doi.org/10.1016/j.drudis . . . 2015.02.008.

BsAb lacking an Fc region rely entirely on their antigen-binding capacity to exert their therapeutic activities. They comprise either the variable VH and VL domains or two antibodies or are based on Fab fragments. For example, one format used in BiTE technology is the genetic fusion of two scFV fragments resulting in tandem scFv molecules so that the two scFv moieties form independent folding units connected in a flexible manner through a peptide linker. [Id., citing Hayden, M S et al. Ther. Immunol. (1994) 1:3-15]. An alternative approach, based on the diabody format, is where the variable domains from two antibodies, A and B, are expressed as two polypeptide chains, VHA-VLB and VHB-VLA, with the domains connected by a short peptide linker, forcing heterodimerization of the two chains. {Id., citing Holliiger, P. ct al. Proc. Natl Acad. Sci. USA (1994) 90:6444-8}. This bivalent diabody format can be further improved by conversion into a single-chain version (scDb) [Id., citing Brusselbach, S. et al. Tumor Target (1999) 4:115-23] and dimeric tetravalent derivatives thereof, so-called ‘tandAb’ molecules with two binding sites for each antigen [Id., citing Kipriyanov, S M et al. J. Mol. Biol. (1999) 293:41-56] as well as disulfide-stabilized variants, such as the dual-affinity retargeting molecules [DART] [Id., citing Johnson, S. et al. J. Mol. Biol. (2010) 399:436-49]. BsAbs also can be generated by fusing different antigen-binding moieties (e.g., scFv or Fab) to other protein domains, which enables further functionalities to be included. For example, two scFv fragments have been fused to albumin, which endows the antibody fragments with the long circulation time of serum albumin [Id., citing Muller, D. et al. J. Biol. Chem. (2007) 282:12650-60; McDonagh, C F et al. Mol. Cancer Ther. (2012) 11:582-593]. Another example is the ‘dock-and-lock’ approach based on heterodimerization of cAMP-dependent protein kinase A and A kinase-anchoring protein [Id., citing Rossi, E A et al. Proc. Natl Acad. Sci. USA (2006) 103:6841-46]. These domains can be linked to Fab fragments and entire antibodies to form multivalent bsAb [Id., citing Rossi, E A et al. Bioconjug. Chem. (2012) 23:309-23].

The term “antibody construct” as used herein refers to a polypeptide comprising one or more the antigen-binding portions of the invention linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art. Antibody portions, such as Fab and F (ab′) 2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

The term “antibody-dependent cellular cytotoxicity” or ADCC, also called antibody-dependent cell-mediated cytotoxicity, is an immune mechanism through which Fc receptor-bearing effector cells can recognize and kill antibody-coated target cells expressing tumor- or pathogen-derived antigens on their surface. It is mediated by the recruitment of cytotoxic effector cells, such as natural killer (NK) cells, macrophages, and polymorphonuclear leukocytes (PMNs), that express Fc gamma receptors (FcγRs) on their surface.

The term “antibody-dependent cellular phagocytosis” or ADCP is a potent mechanism of elimination of antibody-coated foreign particles such microbes or tumor cells. Engagement of FcγRIla and FcγRI expressed on macrophages triggers a signaling cascade leading to the engulfment of the IgG-opsonized particle.

The term “antibody-drug conjugate” or “ADC” as used herein refers to antibodies (e.g., monoclonal antibodies, mAbs) linked to a cytotoxic agent designed to induce target cell death in order to reduce systemic exposure and therefore toxicity of the cytotoxic agent. The linker should be stable in circulation but release the cytotoxic agent once it is delivered to target cells. The unique antigenic target of the antibody component needs to have high expression in the tumor and no or low expression in healthy cells; it should be displayed on the surface of the tumor cell to be available to the circulated antibody; it should possess internalization properties to facilitate the ADS to transport into the cell which will in turn enhance the efficacy of the cytotoxic agent; or exert a “bystander effect.”

The term “antigen” as used herein, is meant to refer to a molecule containing one or more epitopes (either linear, conformational or both) that will stimulate a host's immune-system to make a humoral and/or cellular antigen-specific response. The term is used interchangeably with the terms “immunogen” or “epitope.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

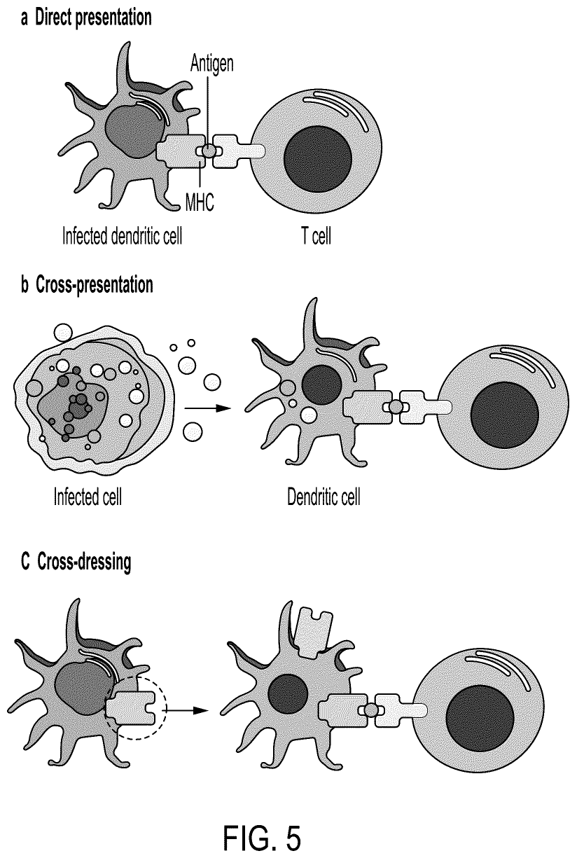

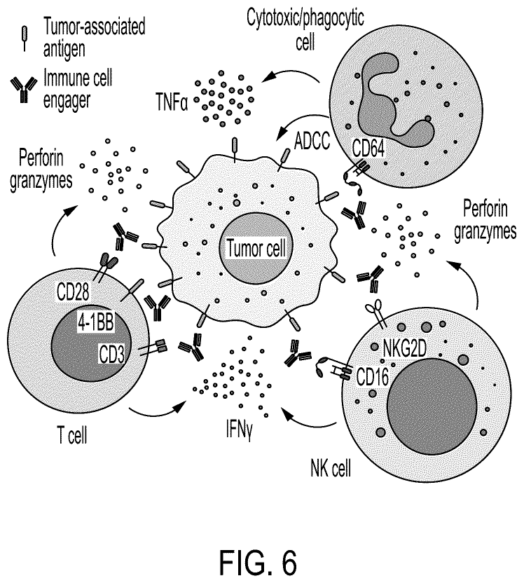

The term “antigen presentation” as used herein, generally refers to the display of antigen on the surface of a cell, e.g., in the form of peptide fragments bound to MHC molecules. FIG. 5 diagrammatically illustrates the pathways to antigen presentation [taken from Yewdell, J W and Dolan, BP. Nature (2011) 471 (7340): 581-82].

As used herein, the term “antigen presenting cell (APC)” refers to a class of cells capable of displaying on its surface (“presenting”) one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bel-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

A Proliferation-Inducing Ligand” or “APRIL” is a TNF-like ligand/cytokine synthesized as a type II transmembrane protein and proteolytically cleaved at a multibasic motif. [Stein, J V et al. April modulates B and T cell immunity. J. Clin. Invest. (2002) 109 (12): 1587-98, citing Schneider, P. et al. J. Exp. Med. (1999) 189:1747-56; Lopez-Fraga, M. et al. EMBO Rep. (2001) 2:945-51; Nardelli, B. et al. Blood (2001) 97:198-204]. It is processed intracellularly by furin convertase prior to its secretion, thus acting solely as a secreted factor. [Id., citing Schneider, P. et al. J. Exp. Med. (1999) 189:1747-56; Lopez-Fraga, M. et al. EMBO Rep. (2001) 2:945-51; Nardelli, B. et al. Blood (2001) 97:198-204] It can bind the receptors TACI and BCMA [Id., citing Gross, J A et al. Nature (2000) 404:995-99; Shu, H B et al. J. Leukoc. Biol. (1999) 65:680-83; Thompson, J S et al. J. Exp. Med. (2000) 192:129-35; Yan, M. et al. Nat. Immunol. (2000) 1:37-41; Xia, X Z et al. J. Exp. Med. (2000) 192:137-43]. Although membrane-bound forms of APRIL are not observed at the cell surface, a fusion protein formed from trans-splicing of TNF-related weak inducer of apoptosis (TWEAK, also known as TNFSF12) and APRIL, known as TWE-PRIL is membrane bound and displays the APRIL receptor binding domain at the cell surface [Gardam, S. and Brink, R. Frontiers Immunol. (2014) 4: article 509, citing Pradet-Balade, B. et al. EMBO J. (2002) 21:2887-95]. TWE-PRIL is biologically active, however its physiological role is yet to be identified. [Id.] APRIL, which is expressed in hematopioctic cells, acts as an in vitro T cell stimulator and signals survival of T cells, which affects the deletion phase of a CD4+ T cell response in vivo. It also is involved in B cell responses.

The term “ARF” (alternative reading frame) as used herein refers to a tumor suppressor protein that accumulates in the nucleolus in response to aberrant oncogenic/hyperproliferative signals and induces cell cycle arrest in G1/S or G2/M transition and apoptosis. Seite, P. (2011) in Schwab, M. (Eds). Encyclopedia of Cancer. Springer, Berlin, Heidelberg. https://doi.org/10.1007/978-3-642-16483-5_384].

The term “B cell receptor” or “BCR” as used herein refers to the antigen-receptor complex of B lineage cells, which is composed of a membrane bound Ig (mlg) monomer plus the Igα/Igβ complex required for intracellular signaling.

B-cell Activation Factor of the TNF Family (BAFF) (also called, BLyS, THANK, TALL-1 and zTNF4) is a TNF-like ligand/cytokine synthesized as a type II transmembrane protein and proteolytically cleaved at a multibasic motif. [Stein, J V et al. April modulates B and T cell immunity. J. Clin. Invest. (2002) 109 (12): 1587-98, citing Schneider, P. et al. J. Exp. Med. (1999) 189:1747-56; Lopez-Fraga, M. et al. EMBO Rep. (2001) 2:945-51; Nardelli, B. et al. Blood (2001) 97:198-204]. It is released from the cell surface by processing of membrane-bound BAFF [Id., citing Schneider, P. et al. J. Exp. Med. (1999) 189:1747-56; Lopez-Fraga, M. et al. EMBO Rep. (2001) 2:945-51; Nardelli, B. et al. Blood (2001) 97:198-204]. It can bind both TACI and BCMA; and a third receptor that is unique to BLysS (BAFF-R or BR-3) [Id., citing Thompson, J S et al. Science (2001) 293:2108-11; Yan, M. et al. Curr. Biol. (2001) 11:1547-52]. BAFF is expressed in hematopoietic cells. Treatment of mice with a soluble decoy form of TACI or BCMA (TACI-Fc or BCMA-Fc) led to reduced B cell numbers and a block in the humoral response [Id., citing Shu, H B et al. J. Leukoc. Biol. (1999) 65:680-3; Yan, M. et al. Nat. Immunol. (2000) 1:37-41; Xia, X Z et al. J. Exp. Med. (2000) 192:137-43; Wang, H. et al. Nat. Immunol. (2001) 2:632-7; Yu, G. et al. Nat. Immunol. (2000) 1:252-6]. These effects have been attributed to BAFF sequestration, as (a) BAFF acts as a costimulator of B cells in the presence of anti-IgM antibodies [Id., citing Moore, P A et al. Science (1999) 285:260-3; Schneider, P. et al. J. Exp. Med. (1999) 189:1747-56; Mukhopadhyay, A. et al. J. Biol. Chem. (1999) 274:15978-81]; (b) in vivo administration of a soluble form of BAFF disrupts spleen architecture due to increased B cell numbers [Id., citing Moore, P A et al. Science (1999) 285:260-3]; (c) mice that express BAFF as a transgene have enlarged spleens and lymph nodes and display autoimmunity due to B cell expansion as a result of increased survival of normally deleted B cells [Id., citing Khare, S D et al. Proc. Natl Acad. Sci. USA (2000) 97:3370-75; Gross, J A et al. Nature (2000) 404:995-99; Mackay, F. et al. J. Exp. Med. (1999) 190:1697-1710]; and (d) BAFF-deficient mice have a phenotype comparable to that of TACI-Fc- or BCMA-Fc-treated mice, i.e., almost complete loss of mature B cells and a severely decreased humoral response [Id., citing Schiemann, B. et al. Science (2001) 293:2111-4; Gross, J A et al. Immunity (2001) 15:289-302]. This last observation suggests that BAFF binding to TACI and/or BCMA is essential for B cell survival and function. Paradoxically, TACI-deficient mice show B cell expansion rather than death, whereas BCMA knockout mice have no overt phenotype [Id., citing Schiemann, B. et al. Science (2001) 293:2111-4; von Bulow, G U et al. Immunity (2001) 14:573-82; Yan, M. et al. Nat. Immunol. (2001) 2:638-43; Xu, S. and Lam, KP. Mol. Cell Biol. (2001) 21:4067-74].

Given the restriction of the expression of BAFF receptors to the lymphoid compartment, it is unsurprising that most identified roles for BAFF are in lymphocytes. The largest body of evidence for the role of BAFF-mediated NFκB2 signaling is in relation to peripheral B cell survival and maturation, the generation of antibody response, and the maintenance of plasma cells. [Gardam, S. and Brink R. Front. Immunol. (2014) 4: article 509]. In addition to providing survival signals to peripheral B cells, BAFF/BAFF-R signaling also is essential for the complete maturation of B cells into a marginal zone phenotype in the spleen. In line with this finding, mouse models with hyperactive NF-κB signaling display an expansion of the MZ B cell population. [Id., citing Mackay, F. et al. J. Exp. Med. (1999) 190:1697-710].

One outcome of NF-κB signaling thought to promote B cell survival is the upregulation of anti-apoptotic molecules, such as Bcl-2 [Gardam, S. and Brink R. Front. Immunol. (2014) 4: article 509, citing Do, R K, et 1. J. Exp. Med. (2000) 192:953-64; Batten, M. et al. J. Exp. Med. (2000) 192:1453-66]. The loss of either BAFF [Gardam, S. and Brink R. Front. Immunol. (2014) 4: article 509, citing Gardam, S. et al. Immunity (2008) 28:391-401], or BAFF-R [Id., citing Gardam, S. et al. Blood (2011) 117:4041-51] can be completely compensated for in terms of B cell survival and maturation by disruption of the TRAF/cIAP ubiquitin ligase complex and thus constitutive hyperactivation of NF-κB2. While other evidence shows that BAFF, BAFF-R and NF-κB2 signaling are all able to individually contribute to B cell survival and maturation, these experiments definitively demonstrate that activation of NF-κB2 sufficiently compensates for loss of BAFF or BAFF-R, i.e., the primary, perhaps even the exclusive purpose of BAFF/BAFF-R signaling in B cells is the activation of NF-κB2 signaling. It is this pathway which facilitates the transcriptional effects required in order for B cells to survive and mature in the periphery.

The term “Bcl-2 family” as used herein refer to a family of intracellular proteins that includes members that promote apoptosis (Bax, Bak, and Bok) and member that inhibit apoptosis (Bcl-2, Bcl-W, and Bcl-XL).

The term “binding” and its various grammatical forms means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “biocompatible” as used herein refers to causing no clinically relevant tissue irritation, injury, toxic reaction, or immunological reaction to living tissue.

The term “biodegradable” as used herein refers to material that will break down actively or passively over time by simple chemical processes, by action of body enzymes or by other similar biological activity mechanisms.

As used herein, the term “biomarker” (or “biosignature”) refers to a peptide, protein, nucleic acid, antibody, gene, metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bone marrow” as used herein refers to the tissue where all cellular elements of the blood-red blood cells, white blood cells, and platelets- are initially generated from hematopoictic stem cells, the site of further B-cell development in mammals, and the source of stem cells that give rise to T cells on migration to the thymus.

The term “CD33” as used herein refers to a sialoadhesin molecule and a member of the Ig supergene family. It is expressed by myeloid stem cells (CFU-GEMM, CFU-GM, CFU-G, and E-BFU), myeloblasts and monoblasts, monocytes/macrophages, granulocyte precursors (with decreasing expression with maturation), and mast cells. Mature granulocytes may show a very low level of CD33 expression. CD33 can be aberrantly expressed on some cases of plasma-cell myeloma. This molecule is not expressed in erythrocytes, platelets, B cells, T cells, or NK cells. CD33 is a myeloid marker and is commonly used for the diagnosis of AML. However, approximately 10-20% of B-lymphoblastic or T-lymphoblastic leukemia/lymphomas may aberrantly express CD33. Faramarz, N. et al. Atlas of Hematopathology, 2d Ed., Chapter 2 Principles of Immunophenotyping (2018) Elsevier, Inc. pp. 29-56.

The term “CD123” as used herein refers to the alpha chain of the interleukin 3 receptor (IL-3R). CD123 is a marker for the identification and targeting of LSCs for refractory or relapsed leukemia. [Shi, M. et al. Cardiovasc. Hematol. Disord. Drug Targets (2019) 19 (30:195-204).

The term “CD135” as used herein refers to the receptor for the cytokine Flt3 ligand (FLT3L). Flt3 ligand is a transmembrane protein that binds to Flt3, a fms-like tyrosine kinase three receptor, and triggers the intracellular signaling cascade that leads to the proliferation and development of hematopoetic stem cells and progenitor cells as well as the development of the immune system. Capitano, M L et al., Reference Module in Neuroscience and Biobehavioral Psychology (2017) doi.org/10.1016/B978-0-12-809324-5.03249-1].

The term “cancer stem cells” as used herein refers to a small number of cells in a tumor with the ability to self-renew and drive tumorigenesis. The stem cell theory of cancer fostering the idea that cancer is primarily driven by a smaller population of stem cells has important implications. For instance, if a therapy does not kill the cancer stem cells within a tumor, chances are that the cancer stem cells will drive the tumor to grow back, often with resistance to the previously used therapy.

The terms “carrier”, excipient, or vehicle” as used herein refer to materials suitable for formulation and administration of pharmaceutically acceptable compositions. The term “carrier” as used herein describes a material that does not cause significant irritation to a subject and does not abrogate the biological activity and properties of the immunotherapeutic agent of the composition of the present disclosure. Carriers must be of sufficiently high purity and of sufficiently low toxicity to render them suitable for administration to the mammal being treated. The carrier can be inert, or it can possess pharmaceutical benefits.

The term “CEBPA” as used herein refers to a gene that provides instructions for making a protein called CCAAT enhancer-binding protein alpha. This protein is a transcription factor involved in the maturation of certain blood cells. It is also believed to act as a tumor suppressor.

The terms “cell line” and “cultured cell line” are used interchangeably to refer to cells of a single type that have been adapted to grow continuously in the laboratory.

As used herein, the term “cell growth” is the process by which cells accumulate mass and increase in physical size. There are many different examples in nature of how cells can grow. In some cases, cell size is proportional to DNA content. For instance, continued DNA replication in the absence of cell division (called endoreplication) results in increased cell size. Megakaryoblasts, which mature into granular megakaryocytes, the platelet-producing cells of bone marrow, typically grow this way. By a different strategy, adipocytes can grow to approximately 85 to 120 μm by accumulating intracellular lipids. In contrast to endoreplication or lipid accumulation, some terminally differentiated cells, such as neurons and cardiac muscle cells, cease dividing and grow without increasing their DNA content. These cells proportionately increase their macromolecule content (largely protein) to a point necessary to perform their specialized functions. This involves coordination between extracellular cues from nutrients and growth factors and intracellular signaling networks responsible for controlling cellular energy availability and macromolecular synthesis. Perhaps the most tightly regulated cell growth occurs in dividing cells, where cell growth and cell division are clearly separable processes. Dividing cells generally must increase in size with each passage through the cell division cycle to ensure that a consistent average cell size is maintained. For a typical dividing mammalian cell, growth occurs in the G1 phase of the cell cycle and is tightly coordinated with S phase (DNA synthesis) and M phase (mitosis). The combined influence of growth factors, hormones, and nutrient availability provides the external cues for cells to grow. [Guertin, D.A., Sabatini, D. M., “Cell Growth,” in The Molecular Basis of Cancer (4th Edn) Mendelsohn, J. et al Eds, Saunders (2015), 179-190].

As used herein, the term “cell proliferation” is meant to refer to the process that results in an increase of the number of cells, and is defined by the balance between cell divisions and cell loss through cell death or differentiation.

As used herein, the term “chemokine” is meant to refer to a class of chemotactic cytokines that orchestrate migration and positioning of immune cells within the tissues. Chemokines bind to seven transmembrane G protein-coupled receptors that trigger intracellular signaling that drives cell polarization, adhesion, and migration [Vilgelm, A E and Richmond, A. Front. Immunol. (2019) doi.org/10.3389/fimmu.2019.00333, citing Griffith, J W et al. Annu. Rev. Immunol. (2014) 32:659-702; Nagarsheth, N. et al. Nat. Rev. Immunol. (2017) 17:559-72]. They are divided into four families based upon structure: CXC, CC, CX3C, and C chemokines. The receptors follow a similar nomenclature system, based upon the family of chemokines to which they bind. In addition there is a family of atypical chemokine receptors that do not directly couple to G proteins, but are reported to have a variety of roles in development, homeostasis, inflammatory disease, infection, and cancer [Id., citing Nibbs, RJ, Graham, GJ. Nat. Rev. Immunol. (2013) 13:815-29].

The term “class switching”, “isotype switching” or “class switch recombination” as used herein refers to a somatic gene recombination process in activated B cells that replaces one heavy chain constant region with one of a different isotype, switching the isotype of antibodies from IgM to IgG, IgA or IgE. This affects the antibody effector functions but not their antigen specificity.

As used herein, the term “cognate help” is meant to refer to a process that occurs most efficiently in the context of an intimate interaction with a helper T cell.

The term “compatible” as used herein refers to the components of a composition are capable of being combined with each other in a manner such that there is no interaction that would substantially reduce the efficacy of the composition under ordinary use conditions.

The terms “complete response” or “complete remission” or “CR” as used herein refer to the disappearance of all signs of cancer in response to treatment. This does not always mean the cancer has been cured.

The term “component” as used herein, is meant to refer to a constituent part, element or ingredient.

The term “composition” as used herein, is meant to refer to a material formed by a mixture of two or more substances.

As used herein, the term “condition” as used herein, is meant to refer to a variety of health states and is meant to include disorders or diseases caused by any underlying mechanism or disorder.

The term “consolidation therapy”, also called “intensification therapy” and “post-remission therapy” as used herein refer to treatment that is given after cancer has disappeared following the initial therapy. Consolidation therapy is used to kill any cancer cells that may be left in the body.

As used herein, the term “contact” and its various grammatical forms is meant to refer to a state or condition of touching or of immediate or local proximity. Contacting a composition to a target destination may occur by any means of administration known to the skilled artisan.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface that have a role in enhancing the activation of a T cell that is already being stimulated through its TCR. For example, HLA proteins, which present foreign antigen to the T cell receptor, require costimulatory proteins which bind to complementary receptors on the T cell's surface to result in enhanced activation of the T cell. The term “co-stimulatory molecules” as used herein refers to highly active immunomodulatory proteins that play a critical role in the development and maintenance of an adaptive immune response (Kaufman and Wolchok eds., General Principles of Tumor Immunotherapy, Chpt 5, 67-121 (2007)). The two signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell unresponsiveness (Id.). Many of the co-stimulatory molecules involved in the two-signal model can be blocked by co-inhibitory molecules that are expressed by normal tissue (Id.). In fact, many types of interacting immunomodulatory molecules expressed on a wide variety of tissues may exert both stimulatory and inhibitory functions depending on the immunologic context (Id.). As used herein the term “co-stimulatory receptor” is meant to refer to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

The term “cross-dressing” as used herein refers to a third pathway for cross-presentation. In cross-dressing, dendritic cells acquire preformed MHC class I molecules in complex with antigens from other cells by the process of trogocytotis (meaning the transfer of cell membrane patches or individual proteins between cells [Yewdell, J W and Dolan, BP, “Cross-dressers turn on T cells”. Nature (2011) 471 (7340): 581-82, citing Joly,. E. and Hudrisier, D. Nature Immunol. (2003) 4:815; Herrera O B et al. J. Imunol. (2004) 173:4828-37] or through gap junctions. This allows antigen presentation by acceptor dendritic cells to occur immediately, without any processing. Cross-dressing is used to activate memory T cells, but not naïve T cells, in response to vial infection [Id., citing Wakins, L M and Bevan, MJ. Nature (2011) 471:629-32].

The term “cross-presentation” as used herein refers to the process by which proteins taken up by dendritic cells from the extracellular milieu can give rise to peptides presented by MNH class I molecules. It enables antigens from extracellular sources to be presented by MHC class I molecules and to activate CD8 T cells.

The term “cross-priming” as used herein refers to activation of CD8 T cells by dendritic cells in which the antigenic peptide presented by MHC class I molecules is derived from an exogenous protein (i.e., by cross-presentation), rather than produced within the dendritic cells directly (cf direct presentation).

The term “culture” and its other grammatical forms as used herein, is meant to refer to a process whereby a population of cells is grown and proliferated on a substrate in an artificial medium.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groα, MCP-1 and TNF-α.

The term “cytotoxic T lymphocytes” (CTLs) as used herein, is meant to refer to effector CD8+T cells. Cytotoxic T cells kill by inducing their targets to undergo apoptosis. They induce target cells to undergo programmed cell death via extrinsic and intrinsic pathways.

The term “CXCR-4” as used herein refers to a G-protein-linked chemokine receptor.

The term “decoy receptor” as used herein refers to receptors that recognize certain inflammatory cytokines with high affinity and specificity, but are structurally incapable of signaling or presenting the agonist to signaling receptor complexes. They act as a molecular trap for the agonist and for signaling receptor components. The interleukin-1 type II receptor (IL-IRII) was the first pure decoy to be identified. Decoy receptors have subsequently been identified for members of the tumor necrosis factor receptor and IL-IR families. [Mantovani, A. et al. Trends in Immunology (92001) 22 (6): 328-36].

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “derived from” as used herein, is meant to encompasses any method for receiving, obtaining, or modifying something from a source of origin.

The term “differentiate” and its various grammatical forms as used herein, are meant to refer to the process of development with an increase in the level of organization or complexity of a cell or tissue, accompanied with a more specialized function.

The term “direct presentation” as used herein refers to the process by which proteins produced within a given cell give rise to peptides presented by MHC class I molecules. This may refer to APCs (such as dendritic cells), or to nonimmune cells that will become the targets of CTLs.

The terms “disease” or “disorder” as used herein refer to an impairment of health or a condition of abnormal functioning.

The terms “disease progression” or “progressive disease” as used herein refers to a cancer that continues to grow or spread.

The term “DNAM-1” (also known as PTA1 or CD226) refers to a 65 000 molecular weight immunoglobulin-like transmembrane glycoprotein that is expressed on the surface of NK cells, T lymphocytes, platelets, monocytes and a subset of B cells. [Xiong, P. et al. Immunology (2015) 146 (3): 369-78, citing Shibuya, A. et al. Immunity (1996) 4:573-81] It is a co-activation receptor involved in the regulation of NK cell adhesion, lymphocyte signaling, lymphokine secretion and cytotoxicity. DNAM-1 contains three domains: an extracellular domain of 230 amino acids, comprising two immunoglobuiln-like domains and eight N-linked glycosylation sites; a transmembrane domain of 28 amino acids; and a cytosolic domain of 60 amino acids, with four putative tyrosine residues and one serine residue for phosphorylation, which recruits signal proteins. CD112 and CD155 are DNAM-1 ligands (DYNAM-1Ls) that belong to the nectin and nectin-like (Necl) protein families, comprising nectin 1-4 and Necl 1-5, respectively. [Id., citing Bottino, C. et al. J. Exp. Med. (2003) 198:557-67; Wang, L. et al. Vet Immunol. Immunopathol. (2009) 132:257-63] DNAM-1 receptor-ligand interactions mediate the cross-talk between NK cells and other immune cells, to maintain homeostasis. [Id., citing Pende, D. et al. Blood (2006) 107:2030-6] DNAM-1 has now been shown to be involved in NK cell education and differentiation, immune synapse formation, cytokine production and cross-talk with DCs and T cells. DNAM-1 also acts synergistically with CD96, TIGIT and CRTAM to regulate NK cell functions. [Id., citing Hou, S. et al. J. Biol. Chem. (2014) 289:6969-77; Nabekura, T. et al. Immunity (2014) 40:225-34; Chan, C J et al. Nat. Immunol (2014) 15:431-8; Martinet, L. et al. Cell Rep. (2015) 11:85-97; Martinet, L. and Smyth, MJ. Nat. Rev. Immunol. (2015) 15:243-54; Pende, D. et al. Blood (2006) 107:2030-6; Lozano, E. et al. J. Immunol. (2013) 191:3673-80].

The term “DNMT3A” as used herein refers to a gene provides instructions for making an enzyme called DNA methyltransferase 3 alpha. This enzyme is involved in DNA methylation, an epigenetic modification. DNMT3A mutations are early events during leukemogenesis and seem to confer poor prognosis to acute myeloid leukemia (AML) patients. Brunetti, L. et al. ColdSpring Harbor Perspect. Med. (2017) 7 (2): a030320].

The term “dose” as used herein, is meant to refer to the quantity of a therapeutic substance prescribed to be taken at one time. The term “maximum tolerated dose” as used herein is meant to refer to the highest dose of a drug or treatment that does not cause unacceptable side effects.

The term “dose escalation study” as used herein refers to a type of study where enrolled patients receive different doses of the drug or investigational agent to determine the recommended phase 2 dose.

The term “dose limiting toxicities” as used herein refers to side effects of a treatment that are serious enough to prevent an increase in dose of that treatment.

The term “ECOG performance status scale” as used herein refers to a scale used to assess how a patient's disease is progressing, assess how the disease affects the daily living abilities of the patient, and determine appropriate treatment and prognosis. The scale, which is shown below in Table 3, was developed by the Eastern Cooperative Oncology Group (ECOG), now part of the ECOG-ACRIN Cancer Research Group, Oken M, Creech R, Tormey D, et al. Toxicity and response criteria of the Eastern Cooperative Oncology Group. Am J Clin Oncol. 1982; 5:649-655.

TABLE 3
ECOG performance status scale.

| Grade | ECOG Performance Status |
|---|---|
| 0 | Fully active, able to carry on all pre-disease performance |
| | without restriction |
| 1 | Restricted in physically strenuous activity but ambulatory and |
| | able to carry out work of a light or sedentary nature, e.g., |
| | light house work, office work |
| 2 | Ambulatory and capable of all selfcare but unable to carry out |
| | any work activities; up and about more than 50% of waking |
| | hours |
| 3 | Capable of only limited selfcare; confined to bed or chair more |
| | than 50% of waking hours |
| 4 | Completely disabled; cannot carry on any selfcare; totally |
| | confined to bed or chair |
| 5 | Dead |

The term “effective dose” as used herein, generally refers to that amount of therapeutic agent, sufficient to induce a therapeutic effect. An effective dose may refer to the amount of the therapeutic agent sufficient to delay or minimize the onset of symptoms. An effective dose may also refer to the amount of the therapeutic agent that provides a therapeutic benefit in the treatment or management of a disease, disorder or condition. Further, an effective dose is the amount with respect to a therapeutic agent alone, or in combination with other therapies, that provides a therapeutic benefit in the treatment or management of a disease. An effective dose may also be the amount sufficient to enhance the subject's (e.g., a human's) own immune response. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

For any therapeutic agent described herein the effective dose may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “eligible subject” as used herein refers to a subject that satisfies the requirements to be treated with the immunotherapy under the professional judgment of the patient's physician. Eligibility criteria may include the subject's age, type and stage of cancer, current health status, medical history, and previous treatments.

The term “exclusion criteria” as used herein refers to specify characteristics that disqualify subjects from clinical studies and often include factors such as comorbidities or concomitant treatment or factors that could mask the effect of the study treatment.

The term “expand” or “amplify” as used herein with respect to cells refers to increasing in cell number.

As used herein, the term “expression” and its other grammatical forms refers to production of an observable phenotype by a gene, usually by directing the synthesis of a protein. It includes the biosynthesis of mRNA, polypeptide biosynthesis, polypeptide activation, e.g., by post-translational modification, or an activation of expression by changing the subcellular location or by recruitment to chromatin.

As used herein the term “Fas” refers to a type 2 membrane protein found on lymphocytes that belongs to the TNF superfamily. In cells that express Fas, engagement of the cell death receptor Fas by Fas ligand (FasL) results in apoptotic cell death, mediated by caspase activation.

As used herein the term “Fc-gamma receptors (FcγRs)” refers to receptors that recognize IgG-coated targets, such as opsonized pathogens or immune complexes (ICs). Cross-linking leads to internalization of the cargo with associated activation of down-stream signaling cascades. FcγRs vary in their affinity for IgG and intracellular trafficking, and therefore have an opportunity to regulate antigen presentation by controlling the shuttling and processing of their cargos. FcγRs bind to the IgG molecule through its Fc (fragment, crystallizable) portion [Junker, F. et al. Front. Immunol. (2020) doi.org/10.3389/fimmu.2020.01393, citing Ravetch, JV, Bolland, S. Annu. Rev. Immunol. (2001) 19:275-90]. In humans, three groups of FcγRs have been described across a variety of cell types: FcγRI, FcγRIIA/B, FcγRIIIA/B [Id., citing Nimmerjahn, F., Ravetch, JV. Nat. Rev. Immunol. (20008) 8:34-47]. These are expressed in differing combinations at the surface membrane of the various immune cells [Id., citing Bruhns, P. Blood (2010) 119:5640-9]. In the case of FcγRI, these include macrophages, neutrophils, eosinophils and DCs. For FcγRIIA, cell types include macrophages, neutrophils, eosinophils, platelets, and Langerhans cells as well as conventional, but not plasmacytoid, DCs [Id., citing Boruchov, A M et al. J. Clin. Invest. (2005) 115:2914-23]. FcγRIIIA is found on natural killer (NK) cells and macrophages, as reviewed elsewhere [Id., citing Hayes, J M et al. J. Inflamm. Res. (2016) 9:2009-19]. The inhibitory Fc gamma receptor FcγRIIB is found on B cells, mast cells as well as macrophages, neutrophils, and cosinophils. Importantly, it is also expressed on cDCs [Id., citing Boruchov, A M et al. J. Clin. Invest. (2005) 115:2914-23]. Flow cytometry experiments suggest that it is unlikely that human pDCs, in contrast to mouse pDCs where expression of the inhibitory receptor FcγRIIB was claimed [Id., citing Flores, M. et al. J. Immunol. (2009) 183:129-39], express any FcγRs [Id., citing Boruchov, A M et al. J. Clin. Invest. (2005) 115:2914-23; Patel, KR Front. Immunol. (2019) 10:223]. Moreover, some studies on pDCs may have included contaminating cDC [Id., citing Balan, S. Int. Rev. Cell Mol. Biol. (2019) 348:1-68]. FcγRIIIB, which can be considered a decoy receptor since it lacks association with downstream signaling molecules (as discussed at later stages of this article), is mainly expressed on neutrophils but may under certain conditions also be expressed on other immune cells like basophils [Id., citing Ravetch, JV, Bolland, S. Annu. Rev. Immunol. (2001) 19:275-90; Bruhns, P. Blood (2012) 119:5640-9].

The term “flow cytometry” as used herein, is meant to refer to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles) (0.5-10° from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The term “FLT-3” or FMS-like tyrosine kinase 3″ as used herein refers to a type III receptor tyrosine kinase that plays an important role in hematopoietic cell survival, proliferation and differentiation. Mutation of the FLT 3 gene is the most frequent genetic alteration and a poor prognostic factor in AML patients.

The term “FLT-3-ITD” as used herein refers to a FLT3 internal tandem duplication. The FLT3-ITD allelic ratio is defined as the ratio of the area under the curve of FLT3-ITD divided by the area under the curve of FLT3-wildtype” using a semi-quantitative DNA fragment analysis. A higher FLT2-ITD AR (generally defined as ≥0.5) is associated with worse survival than lower ratios. [Kantarjiian, H. et al. Blood Cancer J. (2021) 11:41]

The term “germinal center” (“GC”) as used herein refers to sites of intense B-cell proliferation and differentiation that develop in lymphoid follicles of secondary lymphoid organs, such as the spleen and lymh nodes, during an adaptive immune response. Somatic hypermutation and class switching occur in germinal centers. Regulation of GC reactions is critical to ensure high affinity antibody production and to enforce self-tolerance by avoiding emergence of autoreactive B cell clones. This regulation is not a simple outcome of follicular helper T cells/fT follicular regulatory cell balance, but also involves the contribution of other cell types to modulate the GC microenvironment and to avoid autoimmunity. [Stebegg, M. et al. Front. Immunol. (2018) 9:2469].

The term “healthy subject” as used herein refers to a subject having no signs or symptoms of a hematopoietic cancer.

The term “helper T cells” or “TH” cells as used herein refers to effector CD4 T cells that stimulate or “help” B cells to make antibody in response to antigenic challenge. TH2, TH1 and the THF subsets of effector CD4 T cells can perform this function.

The term “IDH-1” or isocitrate dehydrogenase-1″ as used herein refers to the primary source of NADPH reducing equivalents in the cytosol and peroxisomes. Both IDH 1 and IDH2 are important in mitigating cellular oxidative damage induced by intrinsic metabolism and extrinsic factors, like radiation.

The term “intercellular adhesion molecules” or “ICAMs” as used herein refers to cell-adhesion molecules of the immunoglobulin superfamily that bind to the leukocyte integrin CD11a: CD18 (LFA-1). They are crucial in the binding of lymphocytes and other leukocytes to antigen-presenting cells and endothelial cells.

The term “IL-4Ra” as used herein refers to the cytokine-binding receptor chain for IL-4.

As used herein, the term “immune checkpoints” refers to the array of inhibitory pathways necessary for maintaining self-tolerance and that modulate the duration and extent of immune responses to minimize damage to normal tissue. Immune checkpoint molecules such as PD-1, PD-L1, CTLA-4 are cell surface signaling receptors that play a role in modulating the T-cell response in the tumor microenvironment. Tumor cells have been shown to utilize these checkpoints to their benefit by up-regulating their expression and activity. With the tumor cell's ability to commandeer some immune checkpoint pathways as a mechanism of immune resistance, it has been hypothesized that checkpoint inhibitors that bind to molecules of immune cells to activate or inactivate them may relieve the inhibition of an immune response. Immune checkpoint inhibitors have been reported to block discrete checkpoints in an active host immune response allowing an endogenous anti-cancer immune response to be sustained. Recent discoveries have identified immune checkpoints or targets, like PD-1, PD-L1, PD-L2, CTLA4, TIGIT, TIM-3, LAG-3, CCR4, OX40, OX40L, IDO, and A2AR, as proteins responsible for immune evasion.

The terms “immune escape” or “immune evasion” as used herein refers to a strategy to evade a host's immune response. It is characterized by the inability of the immune system to eliminate transformed cells prior to and after tumor development. The host's contribution is manifested by the its inability to recognize antigens expressed by tumor cells, a phenomenon known as “host ignorance.” It happens because of defects in both the innate and adaptive arms of the immune system. The tumor's contribution is manifested by the adaptation of tumor cells to evade the immune systems or by developing a microenvironment that suppresses the immune system. Qian J. et al. (2011) Immune Escape. In: Schwab M. (eds) Encyclopedia of Cancer. Springer, Berlin, Heidelberg. https://doi.org/10.1007/978-3-642-16483-5_2975.

The term “immune homeostasis” refers to the delicate and finely regulated balance of appropriate immune activation and suppression in tissues and organs, driven by a myriad of cellular players and chemical factors. [da Gama Duarte, J. et al. Immunology and Cell Biology (2018) 96:497-506]

The terms “immune response” and “immune-mediated” are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immune phenotype” or “immunotype” as used herein refers to the collective frequency of various immune cell populations and their functional responses to stimuli (cell signaling and antibody responses). [See Kaczorowski, K J et al. Proc. Nat. Acad. Sci. USA (2017) doi/10.1073/pnas. 1705065114]

The terms “immune surveillance” or “immunological surveillance” are used interchangeably to refer to a monitoring process by the immune system to detect and destroy virally infected and neoplastically transformed cells in the body.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunocompromised” as used herein refers to having a weakened immune system and a reduced ability to fight infections and other diseases. The term “immunocompromised” as used herein refers to having a weakened immune system and a reduced ability to fight infections and other diseases. Immunocompromised subjects include patients receiving long-term (>3 months) or high-dose (>0.5 mg/kg/day) steroids or other immunosuppressant drugs, organ or bone marrow transplant recipients, patients with a solid tumor requiring chemotherapy in the last 5 years or with a hematological malignancy whatever the time since diagnosis and who received treatments, patients with leukemia or lymphoma, patients with primary immune deficiency; patients with HIV or AIDS; patients with autoimmune conditions, patients with asthma, which causes the immune system to overreact to harmless substances); patients of advanced age; and smokers.

The term “immunodeficiency” and its other grammatical forms as used herein refers to an inability to produce an adequate immune response because of an insufficiency or absence of antibodies, immune cells, or both. Immunodeficiency disorders can be inherited, such as severe combined immunodeficiency; they can be acquired through infection, such as with HIV; or they can result from chemotherapy.

The term “immunological repertoire” refers to the collection of transmembrane antigen-receptor proteins located on the surface of T and B cells. [Benichou, J. et al. Immunology (2011) 135:183-191)] The combinatorial mechanism that is responsible for encoding the receptors does so by reshuffling the genetic code, with a potential to generate more than 10\hat{ }18 different T cell receptors (TCRs) in humans [Id., citing Venturi, Y. et al. Nat. Rev. Immunol. (2008) 8:231-8] and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. For example, diversity of the TCR gene is generated by rearrangement of the V and J gene segments during T cell development in the thymus. (Makino, Y., et al (1993) J. Exptl Med. 177:1399-1408). The TCR V and J gene segments, like Ig genes, possess recombination signals in which heptamer and nonamer sequences, separated by a 12/23 bp spacer, are flanked by germline V and J gene segments. Id.

The term “immunogen” and its various grammatical forms as used herein is used interchangeably with the term “antigen”.

The terms “immunomodulatory”, “immune modulator”, “immunomodulatory,” and “immune modulatory” are used interchangeably herein to refer to a substance, agent, or cell that is capable of augmenting or diminishing immune responses directly or indirectly, e.g., by expressing chemokines, cytokines and other mediators of immune responses.

The term “immunological synapse” as used herein refers to a highly structured body that functions to concentrate TCR signaling in a defined area. It is associated with the selective recruitment of signaling molecules and exclusion of negative regulators. The synapse is stabilized by a ring of adhesion molecules, including, for example, LFA1, which binds to ICAMI on the APC. For a T cell to become fully active, co-stimulation through a second signaling pathway is required. Many co-stimulators have been identified that share the common characteristic of being transmembrane receptors, often of the TNFR superfamily, that bind transmembrane ligands on the APC. The most important co-stimulator, CD28, binds the ligands CD80 (B7.1) and CD86 (B7.2), both of which are expressed on activated APC. Co-stimulation results in the clonal expansion of CTL with the selected antigen specificity. The expression of CD80/86 is tightly regulated. High-level expression occurs only after an APC receives activation signals, such as inflammatory cytokines, or components of the pathogens such as lipopolysaccharide. Co-stimulation via CD28 is most critical during the initiation of the immune response, as it promotes IL-2 production that, in turn, supports the development of effector T cells. Naïve T cells that receive TCR stimulation in the absence of co-stimulatory signals can become nonresponsive to antigen, a state termed “anergy.” Nutt, S L et al. Clinical Immunology (4th Ed.) chapter 17, Cytotoxic T lymphocytes and natural killer cells. (2013) 215-27.

As used herein, the term “immunostimulatory” and its other grammatical forms refers to augmenting an immune response either directly or indirectly.

As used herein, the term “immunosuppressive” and its other grammatical forms refers to suppressing or diminishing an immune response either directly or indirectly.

The term “immunotherapy” as used herein refers to the measures taken using immunological methods and principles to target the hyper or hyo-immune state of an organism, intervene or adjust the organism's immune function artificially, and strengthen or attenuate the immune response so as to treat disease. It enhances the immune system's ability to recognize, target and eliminate cancer cells in the body. [Zhang, Z. et al. Front. Immunol. (2021) 12: Barbari, C. et al. Intl J. Mol. Sci. (2020) 21:5009]. Some types of immunotherapy only target certain cells of the immune system. Others affect the immune system in a general way. For example, monoclonal antibodies can attach to specific proteins on the surface of cancer cells or immune cells in order to mark the cancer as a target for the immune system, or boost the ability of immune cells to fight the cancer. Cytokine therapy, another example, relies on proteins called interferons and interleukins to trigger an immune response. Interleukin-2 (IL-2) is used to treat kidney cancers and melanomas that have spread to other regions of the body. Interferon alpha (IFN-alpha) is currently being used to treat melanoma, kidney cancer and certain leukemias and lymphomas. These cytokine treatments are also being combined with other types of immunotherapies to increase their effectiveness.

The term “immunotherapeutic agent” as used herein refers to a drug, molecule, nucleic acid, protein, composition or cell that provides a therapeutic effect. The term “active” as used herein refers to the ingredient, component or constituent of the compositions of the present invention responsible for the intended therapeutic effect. The terms “immunotherapeutic agent” and “active agent” are used interchangeably herein.

The term “immunotherapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

The term “induction therapy”, also called first line therapy, primary therapy and primary treatment, refers to the first treatment given for a disease.

The term inhibitor receptor lymphocyte activation gene-3 or “LAG-3” as used herein refers to a member of the immunoglobulin superfamily (IgSF) and binds to major histocompatibility complex (MHC) class II. LAG-3 expression on TILs is associated with tumor-mediated immune suppression.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Lymphocytes are much more common in the lymphatic system, and include B cells, T cells, killer T-cells, and natural killer (NK) cells. There are two broad categories of lymphocytes, namely T cells and B cells. T-cells are responsible for cell-mediated immunity whereas B-cells are responsible for humoral immunity (relating to antibodies). T-cells are so-named such because these lymphocytes mature in the thymus; B-cells mature in bone marrow. B cells make antibodies that bind to pathogens to enable their destruction. CD4+ (helper) T cells coordinate the immune response. CD8+ (cytotoxic) T cells and Natural Killer (NK) cells are able to kill cells of the body that are, e.g., infected by a virus or display an antigenic sequence.

The term “lymphocyte activation” refers to stimulation of lymphocytes by specific antigens, nonspecific mitogens, or allogeneic cells resulting in synthesis of RNA, protein and DNA and production of lymphokines, the soluble product of lymphocytes; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Full responsiveness of a T cell requires, in addition to receptor engagement, an accessory cell-delivered costimulatory activity, e.g., engagement of CD28 on the T cell by CD80 and/or CD86 on the antigen presenting cell (APC).

The term “macrophage” as used herein refers to a mononuclear, actively phagocytic cell arising from monocystic stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules.

The term “maintenance therapy” or “continuous therapy” as used herein refers to the ongoing treatment cancer after it has responded to induction therapy to prevent relapse.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups-class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP1: TAP2 peptide transporter, the PSMB (or LMP) genes that encode proteasome subunits, the genes encoding the DMα and BMβ chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeway's Immunobiology. 9th ed., GS, Garland Science, Taylor & Francis Group, 2017. pps. 232-233]. In humans, there are three MHC class II isotypes: HLA-DR, HLA-DP, and HLA-DQ, encoded by a and B chain genes within the Human Leukocyte Antigen (HLA) locus on chromosome 6 [Wosen, J E et al. Front. Immunol. (2018) doi.10.3389/fimmu.2018.02144].

The term “MHC restriction” as used herein refers to the requirement that APCs or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

As used herein, the terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “mediated” and its various grammatical forms as used herein refers to depending on, acting by or connected through some intervening agency.

The term “memory cells” as used herein refers to B and T lymphocytes generated during a primary immune response that remain in a quiescent state until fully activated by a subsequent exposure to specific antigen (secondary immune response). Memory cells generally are more sensitive than naïve lymphocytes to antigen and respond rapidly on reexposure to the antigen that originally induced them. During an immune response, naïve T cells (TN) are primed by antigen-presenting cells (APCs). Depending on the strength and quality of stimulatory signals, proliferating T cells progress along a differentiation pathway that culminates in the generation of terminally differentiated short-lived effector T (TEFF) cells. When antigenic and inflammatory stimuli cease, primed T cells become quiescent and enter into the memory stem cell (TSCM), central memory (TCM) cell or effector memory (TEM) cell pools, depending on the signal strength received. TSCM cells possess stem cell-like attributes to a greater extent than any other memory lymphocyte population. Although both TCM and TEM cells can also undergo self-renewal, the capacity to form diverse progeny is progressively restricted, so that only TSCM cells are capable of generating all three memory subsets and TEFF cells; TCM cells can give rise to TCM, TEM and TEFF cells, and TEM cells can only produce themselves and TEFF cells. [Gattinoni, L. et al. Nature Revs. Cancer 12 (2012) 671-84].

As used herein, the terms “mixed-phenotype acute leukemia” or “MPAL” are used herein to refer to a heterogeneous group of leukemias in which assigning a single lineage of origin is not possible. It is currently defined by a limited set of lineage-specific markers proposed in the 2008 WHO monograph on classification of tumors of hematopoietic and lymphoid tissues. [Wolach, O. and Stone, RM. Blood (2015) 125 (16): 2477-85].

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “myeloblast” or “blasts” as used herein refers to a type of immature white blood cell. Myeloblasts have a diameter of 10-20 μm, are derived from HSCs and are normally found in the bone marrow. Myeloblasts become mature granulocytes (neutrophils, basophils, and cosinophils).

The term “myeloid” as used herein refers to the lineage of blood cells that develop during myelopoiesis and include granulocytes, monocytes, megakaryocytes and dendritic cells. Circulating erythrocytes and platelets also develop from myeloid progenitor cells.

The term “myelopoiesis” as used herein refer to the development of non-lymphoid leukocytes. Myelopoiesis begins with the differentiation of a small population of pluripotent stem cells to colony forming unit that generates myeloid cells (CFU-GEMM) and then to the committed primitive myeloid precursors, granulocyte/macrophage colony-forming units (CFU-GM). This process requires myelopoietic cytokines (GM-CSF, SCF, IL-3, and IL-6). CFU-GM give rise to more mature colony-forming units CFU-G, CFU-M, CFU-Eo, and CFU-Baso which in turn differentiate into neutrophils, macrophages, cosinophils, and basophils, respectively.

The term “myeloproliferative neoplasms” or “MPNs”, previously termed the myeloproliferative disorders, are characterized by the clonal proliferation of one or more hematopoietic cell lineages, predominantly in the bone marrow, but sometimes in the liver and spleen. In contrast to myelodysplastic syndromes (MDS), MPNs demonstrate terminal myeloid cell expansion into the peripheral blood. MPNs include chronic myelogenous leukemia (CML), chronic neutrophilic leukemia, polycythemia vera (PV), primary myelofibrosis (PMF), essential thrombocythemia (ET), chronic cosinophilic leukemia, mastocytosis, and unclassifiable MPNs.

The term “naïve T cell” as used herein refers to a T cell that has not previously been exposed to an antigen. Naïve T cells are conventionally defined by coexpression of the RA isoform of the transmembrane phosphatase CD45, the lymph node homing molecules L-selectin (CD62L) and CCR7, and the costimulatory receptors CD27 and CD28. [De Rosa, S C et al. Nature Med. (2001) 7:245-48].

Nectins and nectin-like molecules (Necls) have recently emerged as cell adhesion molecules that have a variety of cellular functions, including cell movement, proliferation, differentiation, polarization and survival, as well as cell-cell adhesion. [Takai, Y. et al. Nature Rev. Molec. Cell Biol. (2008) 9:603-15].

The term “natural killer (NK) cells” as used herein is meant to refer to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

The abbreviation “NFκB” as used herein refers to a proinflammatory transcription factor that switches on multiple inflammatory genes, including cytokines, chemokines, proteases, and inhibitors of apoptosis, resulting in amplification of the inflammatory response [Barnes, PJ, (2016) Pharmacol. Rev. 68:788-815]. The molecular pathways involved in NF-κB activation include several kinases. The classic (canonical) pathway for inflammatory stimuli and infections to activate NF-κB signaling involve the IKK (inhibitor of KB kinase) complex, which is composed of two catalytic subunits, IKK-α and IKK-β, and a regulatory subunit IKK-γ (or NFκB essential modulator [Id., citing Hayden, MS and Ghosh, S (2012) Genes Dev. 26:203-234]. The IKK complex phosphorylates Nf-κB-bound IκBs, targeting them for degradation by the protcasome and thereby releasing NF-κB dimers that are composed of p65 and p50 subunits, which translocate to the nucleus where they bind to KB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-α homodimers and releases RelB and processes p100 to p52 in response to certain members of the TNF family, such as lymphotoxin-β [Id., citing Sun, SC. (2012) Immunol. Rev. 246:125-140]. This pathway switches on different gene sets and may mediate immune functions different from the canonical pathway. Dominant-negative IKK-β inhibits most of the proinflammatory functions of NF-κB, whereas inhibiting IKK-α has a role only in response to limited stimuli and in certain cells, such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151:179-89]. Activation of non-canonical NF-κB signaling in response to B cell activating factor of the TNF family (BAFF) contributes to key events throughout the lifespan of a B cell. These include facilitating the survival of immature B cells in the periphery and activating transcriptional programs which allow them to mature into follicular and marginal zone (MZ) B cells; contributing to some T-independent immune responses; extending the duration of germanal center (GC) reactions, and maintaining long-lived plasma cells in the BM. [Gardam, S. and Brink, R. Front. Immunol. (2014) 4: article 509].

The term “NKG2D” as used herein refers to an activating receptor expressed by all NK cells and subsets of T cells (γδ T cells, CD8+ T cells and CD4+T cells) in humans. It is encoded by the KLRK1 gene (killer cell lectin-like receptor subfamily K, member 1) . . . . NKG2D receptor functions as an activating receptor by virtue of its interactions with the signaling adaptor dimer DAP10 in humans and with DAP10 and DAP12 in mice (Raulet, D H et al. Annu. Rev. Immunol. (2013) 31:4123-41, citing Champsaur, M. and Lanier, LL. Immunol. REcv. (2010) 235:267-85; Wu, J. et al. Science (1999) 285:730-32). When the receptor is ligated, DAP10 provides signals that recruit the p85 subunit of phosphatidylinositol 3-kinase (PI3K) and a complex of GRB2 and VAV1. Engagement of NKG2D on NK cells induces degranulation and cytokine production.

NK cell activation as a result of NKG2D engagement can modify, or be modified by, engagement of other NK receptors. For naïve human NK cells, synergistic activation occurs when NKG2D is coengaged with 2B4, a SLAM family receptor whose ligand is broadly expressed by hematopoietic cells, or with NKp46, another activating receptor (Id., citing Bryceson, Y T et al. Blood (2006) 107:159-6611) 11). Conversely, NKG2D-induced NK activation can be inhibited (albeit not necessarily completely) if the target cell expresses MHC class I molecules that engage inhibitory receptors on NK cells, such as KIRs (killer cell immunoglobulin-like receptors) in humans [Id., citing Jamieson, A M et al. Immunity (2002) 17:19-291 Rgunathan, J. et al., Blood (2005) 105:2133-40].

NKG2D binds to several different ligands, all of which are homologous to MHC class I molecules but have no known role in antigen presentation [Id., citing Raulet, DH. Nat. Rev. Immunol. (2003) 3:781-90; Champsaur, M. and Lanier, LL. Immunol. Rev. (2010) 235:267-85; Eagle, R A and Trowsdale, J. Nat. Rev. Immunol. (2007) 7:737-44; Machuldova, A. et al. Front. Immunol. (2021) 12:651751, citing Stephens, HA. Trends Immunol. (2001) 22 (7): 378-85]. Like MHC proteins, they exhibit considerable allelic variation. In humans, the NKG2D ligands include MHC class I chain-related protein A (MICA) and MHC class I chain-related protein B (MICB), both encoded by genes in the MHC, and up to six different proteins called Unique long (UL) 16-binding proteins (ULBPs), also known as retinoic acid early transcript 1 (RAET1) proteins. Like MHC proteins, the NKG2D ligands exhibit considerable allelic variation.

All NKG2D ligands are encoded by distinct genes in the host's own genome, i.e., the ligands are self-proteins. NKG2D ligands are expressed poorly or not at all by most normal cells but are upregulated in cancer cells and virus-infected cells. This type of recognition process, in which self-coded ligands for activating receptors are induced on unhealthy cells, has been termed “induced self recognition [Id. citing Diefenbach, A. and Raulet, DH. Immunol. Rev. (2001) 181:170-84], which is distinct from “missing self recognition”, a phenomenon in which loss of MHC ligands for NK inhibitory receptors sensitizes cells for elimination by NK cells. Various cellular pathways activated as a result of cellular stress, infection, or tumorigenesis regulate expression of the NKG2D ligands.

The structures of NKG2D-ligand complexes indicate that NKG2D binds diagonally over the α1 and α2 helices of the ligands, much as T-cell receptors bind over MHC molecules. Despite the poor homology of different ligands, some of the key residues that interact with NKG2D are conserved, and the NKG2D residues involved in binding are similar in the different structures. NKG2D ligands are generally poorly expressed by normal cells, but are upregulated in transformed, infected and, in some cases, stressed cells.

The engagement of NKG2D is a sufficient stimulus to activate cytolysis and cytokine production by NK cells. However, it provides an enhancing or co-stimulatory signal for the activation of CD8+ T cells and probably other T cells.

The term “neoantigen” as used herein refers to tumor-specific antigens generated by mutations in tumor cells, which are only expressed in tumor cells.

The term “neoepitope” as used herein refers to tumor-specific MHCI restricted epitopes.

The term “neutrophils” or “polymorphonuclear neutrophils (PMNs)” as used herein refers to the most abundant type of white blood cells in mammals, which form an essential part of the innate immune system. They form part of the polymorphonuclear cell family (PMNs) together with basophils and cosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis, meaning the directed motion of a motile cell or part along a chemical concentration gradient toward environmental conditions it deems attractive and/or away from surroundings it finds repellent.

The term “non-expanded” as used herein, is meant to refer to a cell population that has not been grown in culture (in vitro) to increase the number of cells in the cell population.

The abbreviation “NPM1” as used herein refers to a mutation of nucleophosmin-1. NPM1 provides instructions for making nucleophosmin found in the nucleolus where it attaches to ARF, which prevents cells from growing and dividing in an uncontrolled way.

The term “objective response rate” or “ORR” as used herein refers to the percentage of people in a study or treatment group who have a partial response or complete response to the treatment within a certain period of time.

The term “overall survival” as used herein refers to the length of time from either the date of diagnosis or the start of treatment for a disease, such as cancer, that patients diagnosed with the disease are still alive.

The term “paraprotein” as used herein refers to monoclonal immune globulin fragments or intact immune globulins produced by usually a malignant cone of plasma cells or B cells. These proteins are associated with a spectrum of kidney disorders caused by either direct effects on the kidney cells or deposition in various kidney cells. These disorders are now classified as monoclonal gammopathy of renal significance (MGRS).

The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), or infusion techniques.

The term “peptide” is used herein to designate a series of amino acid residues, connected one to the other typically by peptide bonds between the alpha-amino and carbonyl groups of the adjacent amino acids. Peptides are typically 9 amino acids in length, but can be as short as 8 amino acids in length, and as long as 14 amino acids in length. A series of amino acids are considered an “oligopeptide” when the amino acid length is greater than about 14 amino acids in length, typically up to about 30 to 40 residues in length. When the amino acid residue length exceeds 40 amino acid residues, the series of amino acid residues is termed a “polypeptide”.

As used herein, the term “perforin” is meant to refer to a molecule that can insert into the membrane of target cells and promote lysis of those target cells. Perforin-mediated lysis is enhanced by enzymes called granzymes.

The terms “peripheral blood mononuclear cells” or “PBMCs” are used interchangeably herein to refer to blood cells having a single round nucleus such as, for example, a lymphocyte or a monocyte. PBMCs are a critical component in the immune system's responses to infections.

The term “plasma cell” as used herein refers to terminally differentiated B cells that secrete antibody. They may be short-lived, with no isotype switching or somatic hypermutation, or long lived, meaning they undergo isotype switching and somatic hypermutation.

Plasma cell differentiation. In the bone marrow and fetal liver, precursor B lineage cells differentiate into immature B cells as they pass through a series of developmental steps that are marked by the sequential rearrangement of the immunoglobulin (Ig) heavy (H) and light (L) chain gene loci, the expression of H chain and L chain proteins, their association to create complete Ig molecule, and tests of the function of membrane-bound surface IgM (mlgM), which together with Igα and Igβ form the B-cell receptor (BCR) for antigen [Vale, A M et al. Development and function of B cell subsets. Chapter 7 in Molecular Biology of B cells, 2d Ed. Frederick W. Alt, Tasuku Honjo, Andreas Radbruch, Michael Reth, Eds., Elsevier/Academic Press, New York London (2015) pp. 99, citing Hardy, R R and Hayakawa, K. Annu. Rev. Immunol. (2001) 19:595-621; Hardy, R. R. in. Chapter 8, B. Lymphocyte Development and Biology, Fundamental Immunology, Paul, W. Ed. 7th Ed. Wolters Kluwer (2013) Philadelphia 215-45]. As these newly formed immature B cells leave the tissues of their birth, they are subject to further selection steps as they enter into the pool of mature B lymphocytes. [Id., citing Rolink, A G et al. Immunol. Rev. (2004) 197:41-50] These steps involve a series of developmental programs that include checkpoints to evaluate the composition, specificity and reactivity of the BCR carried by each individual lymphocyte [Id., citing Nemazee, D. and Weigert, M. J. Exp. Med. (2000) 191:1813-17; Nemazee, D. Nat. Rev. Immunol. (2006) 6:728-40]. These programs and checkpoints eventually result in the creation of an array of mature B cells that display a diverse BCR repertoire that can react to a broad range of both ancient and novel antigens [Id., citing Goodnow, CC. Ann. N.Y. Acad. Sci. (1997) 815:55-66; Rolink, A. and Melchers, F. Immunol. Lett. (1996) 54:157-61; Osmond, D C et al. Immunology Today (1998) 19:65-8]. Cells within this B cell pool can be segregated into subsets of mature IgM-bearing B lymphocytes on the basis of characteristic phenotypic differences in surface molecule expression, differences in anatomic location, and differences in responses to immunologic stimuli.

B cells activated by antigen and receiving appropriate additional stimuli through antigen, T cell contact, and/or cytokines will initiate proliferation, in which the cells enlarge and appear as blast cells. Among antibody-secreting cells, plasmablasts represent a clear stage of proliferating B cells secreting antibody [Tatlinton, D. Chapter 14b. “Plasma Cell Biology” in Molecular Biology of B cells, 2d Ed. Frederick W. Alt, Tasuku Honjo, Andreas Radbruch, Michael Reth, Eds., Elsevier/Academic Press, New York London (2015) pp. 232-35.

Plasmablasts (PB) are B cells in a lymph node that already shows some features of a plasma cell. They express many markers of the B cell lineage, including the BCR, co-stimulatory molecules such as B220 (a B-cell restricted isoform of CD45) and CD80/86, and the mechanism for antigen presentation through expression of major histocompatibility complex II, PB secreting each of the Ig isotypes are detectable [Id., citing Oracki, S A et al. Immunol. Rev. (2010) 237:140-59. PBs are inherently short lived irrespective of the nature of the antigen.

Plasma cells are found in the medulla of the lymph nodes, in splenic red pulp, in bone marrow and in mucosal tissues and are post-mitotic antibody secreting cells. The transformation from B cell to plasma cell requires a dramatic change in the transcription program of the cell [Id., citing Nutt, S L, et al. Semin. Immunol. (2011) 23:341-9] whereby the B cell has to silence genes that define B cell identity and function and express the corresponding genes for plasma cell identity and function. Each state is relatively stable and maintained by master regulators, such as Pax5 and Bcl6 for the B cell state and interferon regulatory factor 4 (IRF4) and Blimp1 for the plasma cell state. Although several models have been proposed, no one model has been identified that explains key feature in the molecular regulation of plasma cell differentiation

Blimp-1 acts primarily as a transcriptional repressor; among its targets are Pax5 and Bc16, genes that sustain B cell identity in general and in germinal centers, respectively.

Plasma cells are intrinsically short lived—for example, if removed from their in vivo location and placed in culture in vitro, most die within a day. Factors that can sustain plasma cells include soluble and membrane-bound factors. IL6, TNF-alpha, CXCL12, APRIL and BAFF all have the ability to support plasma cell viability, as do VEGF-1 and ICM-1 binding, respectively, to very late antigen-4 (VLA-4) (an a4β1 integrin that is a leukocyte ligand for VCAM-1, fibronectin and osteopontin involved in the acquisition of antigen by B cells and their subsequent activation, lowering the activation threshold) and lymphocyte function-associated antigen-1 [LFA-1] (a member of the heterodimeric B2 integrin family), and hyaluronic acid and fibronectin binding to CD44. In addition, there are survival factors that are considered to operate in inflammatory situations, specifically CXCL9, 10 and 11 binding to CXCR3 [Id., citing Winter, O. et al. J. Immunol. (2012) 189:5105-11]. The T cell costimulatory molecule CD28 has been identified as providing survival signals to plasma cells by binding its ligands CD80/86 [Id., citing Rozanski, C H et al. J. Exp. Med. (2011) 208:1435-46]. CD28 is repressed in B cells by Pax5, so its early re-expression in plasma cells is fully consistent with their loss of Pax5 activity [Id., citing Delogu, A. et al. Immunity (2006) 24:269-81].

The term “plasmablasts” as used herein refer to proliferating progeny of an activated B cell. Plasmablasts become plasma cells. Antigen binding to the BCR triggers activation of Src family kinases such as Lyn and Fyn leading to phosphorylation of Igα (CD79a) and Igβ (CD79b), recruitment of Syk kinase and subsequent recruitment and phosphorylation of BLNK, Btk and PLCγ [Luo, W. et al. J. Immunol. (2014) 193 (2): 909-20, citing Packard, TA & Cambier, JC. F1000 prime reports (2013) 5:40]. These events activate the Ras pathway, PKC pathway and calcium flux, eventually triggering the activation of NF-κB, Erk and JNK. These positive signals are normally counterbalanced by negative signals that limit B cell activation and prevent spontaneous B cell proliferation and differentiation to plasma cells [Id., citing Nitschke, L. Curr. Opin. Immunol. (2005) 17:2990-97]. Negative signals are generated by a series of membrane receptors (CD22, CD72, FcγRIIb, PIR-B, Siglec-G, etc.) that are phosphorylated by Lyn. This allows them to recruit phosphatases such as SHP1 and SHIP1 that reverse phosphorylation of signaling molecules in the BCR pathway and dampen BCR signaling [Id., citing Poe, JC & Tedder, TF, Trends Immunol. (2012) 33:413-20; Tsubata, T. Infectious disorders drug targets (2012) 12:181-90; Vang, T. et al. Annu. Rev. Immunol. (2008) 26:29-55].

The term “priming” as used herein refers to the first encounter with a given antigen, which generates a primary adaptive immune response. The term “unprimed cells” (also referred to as virgin, naïve, or inexperienced cells) as used herein refers to T cells and B cells that have generated an antigen receptor (TCR for T cells, BCR for B cells) of a particular specificity, but have never encountered the antigen. For example, before helper T cells and B cells can interact to produce specific antibody, the antigen-specific T cell precursors must be primed.

Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. [Janeway, CA, Jr., “The priming of helper T cells, Semin. Immunol. (1989) 1 (1): 13-20]. Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

The term “progression” as used herein refers to the course of disease as it becomes worse or spreads in the body.

The term “progression-free survival” or PFS″ as used herein refers to the length of time during and after the treatment of the disease that a patient lives with the disease but it does not get worse.

The term “proliferate” and its various grammatical forms as used herein is meant to refer to the process that results in an increase of the number of cells, and is defined by the balance between cell division and cell loss through cell death or differentiation.

The term “proteasome” as used herein refers to a multicatalytic proteinase complex, critical for regulated degradation of cellular proteins to peptides by the ubiquitin proteasome system (UPS).

The term “recurrent cancer” or “recurrence” means a cancer that has come back, usually after a period of time during which the cancer could not be detected. The cancer may come back to the same place as the primary tumor or to another place in the body.

The term “refractory cancer” or “resistant cancer” means a cancer that does not respond to treatment. The cancer may be resistant at the beginning of treatment or it may become resistant during treatment.

The term “relapse” refers to the return of a disease or the signs and symptoms of a disease after a period of improvement.

The term “relapse-free survival” (RFS) or “disease-free survival” (DFS) means the length of time after primary treatment for a cancer ends that the patient survives without any signs or symptoms of that cancer.

The term “RUNX1” as used herein refers to a gene that provides instructions for making runt-related transcription factor 1 (RUNX1). The RUNX1 transcription factor binds to specific regions of DNA and helps control the activity of particular genes by interacting with core binding factor beta (CBFB), a protein produced from the CBFB gene, which helps RUNX1 bind to DNA and prevents it from being degraded. The RUNX1 transcription factor activates genes that help control hematopoitic stem cell emergence and regulation. [Swiers, G. et al. Int. J. Deve. PBiol. (2010) 54 (6-7): 1151-63]. The transcription factor RUNX1 is the fusion partner of RUNXIT1 (ETO) in the recurring t (8;21) (q22;q22) translocation present in 8-13% of adult patients with de novo acute myeloid leukemia (AML). [Greif, P A et al. Haematologica (2012) 97 (120): 1909-15]

The term “sign” as used herein refers to a healthcare provider's evidence of disease.

The term “SLAM (signaling lymphocyte activation molecule” as used herein refers to a family of related cell-surface receptors that mediate adhesion between lymphocytes, that includes SLAM, 2B4, CD84, LLy 106, Ly9 and CRACC.

The term “standard of care” as used herein refers to treatment for a disease that is accepted and widely used by doctors.

As used herein, the term “stimulate” in any of its grammatical forms as used herein is meant to refer to inducing activation or increasing activity.

The term “stimulate an immune cell” or “stimulating an immune cell” as used herein is meant to refer to a process (e.g., involving a signaling event or stimulus) causing or resulting in a cellular response, such as activation and/or expansion, of an immune cell, e.g. a CD8+T cell.

The terms “subject” or “individual” or “patient” are used interchangeably to refer to a member of an animal species of mammalian origin, including but not limited to, mouse, rat, cat, goat, sheep, horse, hamster, ferret, pig, dog, guinea pig, rabbit and a primate, such as, for example, a monkey, ape, or human.

The phrase “a subject with acute myeloid leukemia” as used herein refers to a subject who presents with diagnostic markers and/or symptoms associated with acute myeloid leukemia or who has been diagnosed with acute myeloid leukemia.

The phrase “subject in need thereof” as used herein refers to an eligible patient that (i) will be administered an immunotherapy according to the present disclosure, (ii) is receiving at least one immunotherapy according to the present disclosure; or (iii) has received at least one immunotherapy according to the present disclosure, unless the context and usage of the phrase indicates otherwise.

The term “symptom” as used herein refers to a patient's subjective evidence of disease.

The terms “T cell” or “T lymphocyte” or are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC).

Although the lineage relationship between T cell subsets remains controversial, T cells cluster in populations that can be arranged as a progressive continuum on the basis of phenotypic, functional and transcriptional attributes. T lymphocytes transition through progressive stages of differentiation that are characterized by a stepwise loss of functional and therapeutic potential in the order from naïve T (TN) cells to T memory stem cells (TSCM) (the most immature antigen experienced T cells), to T central memory (TCM) cells, which patrol central lymphoid organs, to Teffector memory (TEM) cells, which patrol peripheral tissues. In contrast to TN cells, memory T cells are capable of rapidly releasing cytokines on restimulation. TCM cells more efficiently secrete IL-2 and TEM have an increased capacity for IFNγ release and cytotoxicity. All antigen-experienced T cells upregulate the common IL-2 and IL-15B receptor (IL-2RB) conferring the ability to undergo homeostatic proliferation in response to IL-15, and also display high amounts of CD95 (also known as FAS), a receptor that provides either costimulatory or pro-apoptotic signals depending on the efficiency of CD95 signaling complex formation and on which particular intracellular signaling proteins are part of the complex. [Gattinoni, L. et al. Natur Revs. Cancer 12:671-684].

The term “T cell antigen” as used herein is meant to refer to a protein or fragment thereof which can be processed into a peptide that can bind to either Class I MHC, Class II MHC, non-classical MHC, or CDI family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell exhaustion” as used herein refers to a state of T cell dysfunction that arises during many chronic infections and cancer. It is defined by poor effector function, sustained expression of inhibitory receptors and a transcriptional state distinct from that of functional effector or memory T cells. Modulating pathways overexpressed in exhaustion—for example, by targeting programmed cell death protein 1 (PD1) and cytotoxic T lymphocyte antigen 4 (CTLA4)—can reverse this dysfunctional state and reinvigorate immune responses [Wherry E J and Kurachi, M. Nature (2015) 15:486-99, citing Wherry E J. Nat. Immunol. (2011) 131:492-499; Schietinger A, Greenberg P D. Trends Immunol. (2014) 35:51-60; Barber D L, et al. Restoring function in exhausted CD8 T cells during chronic viral infection. Nature. (2006) 439:682-687; Nguyen L T, Ohashi P S. Nat. Rev. Immunol. (2014) 15:45-56]. The level and duration of chronic antigen stimulation and infection seem to be key factors that lead to T cell exhaustion and correlate with the severity of dysfunction during chronic infection. Examples of inhibitory receptors include the inhibitory pathways mediated by PDI in response to binding of PD1 ligand 1 (PDL1) and/or PDL2. [Id., citing Okazaki T, et al., Nature Immunol. (2013) 14:1212-1218, Odorizzi P M, Wherry E J. J. Immunol. (2012) 188:2957-2965, Araki K, et al. Cold Spring Harb. Symp. Quant. Biol. (2013) 78:239-247]. Exhausted T cells can co-express PDI together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D, et al. Nat. Immunol. (2009) 10:29-37]. Typically, the higher the number of inhibitory receptors co-expressed by exhausted T cells, the more severe the exhaustion. It has been suggested that inhibitory receptors such as PDI might regulate T cell function in several ways [Id., citing Schietinger A, Greenberg P D. Trends Immunol. (2014) 35:51-60; Odorizzi P M, Wherry E J. J. Immunol. (2012) 188:2957-2965], e.g., by ectodomain competition, which refers to inhibitory receptors sequestering target receptors or ligands and/or preventing the optimal formation of microclusters and lipid rafts (for example, CTLA4); second, through modulation of intracellular mediators, which can cause local and transient intracellular attenuation of positive signals from activating receptors such as the TCR and co-stimulatory receptors [Id., citing Parry R V, et al. Molec. Cell. Biol. (2005) 25:9543-9553; Yokosuka T, et al. J. Exp. Med. (2012) 209:1201-1217; Clayton K L, et al. J. Immunol. (2014) 192:782-791]; and third, through the induction of inhibitory genes [Id., citing Quigley M, et al. Nat. Med. (2010) 16:1147-1151]. Co-stimulatory receptors also are involved in T cell exhaustion [Id., citing Odorizzi P M, Wherry E J. J. Immunol. (2012) 188:2957-2965] . . . . It has also been possible to exploit the potential beneficial role of co-stimulation to reverse exhaustion by combining agonistic antibodies to positive co-stimulatory pathways with blockade of inhibitory pathways. 4-1BB (also known as CD137 and TNFRSF9) is a TNFR family member and positive co-stimulatory molecule that is expressed on activated T cells. Combining PDI blockade and treatment with an agonistic antibody to 4-1BB dramatically improved exhausted T cell function and viral control [Id, citing Vezys V, et al. J. Immunol. (2011) 187:1634-1642]. Soluble molecules are a second class of signals that regulate T cell exhaustion; these include immunosuppressive cytokines such as IL-10 and transforming growth factor-β (TGFβ) and inflammatory cytokines, such as type I interferons (IFNs) and IL-6. [Id.]

The term “T cell mediated immune response” as used herein is meant to refer to a response that occurs as a result of recognition of a T cell antigen bound to an antigen presenting molecule on the cell surface of an APC, coupled with other interactions between costimulatory molecules on the T cell and APC. This response serves to induce T cell proliferation, migration, and production of effector molecules, including cytokines and other factors that can injure cells.

The term “T cell receptor” (TCR) as used herein, is meant to refer to a complex of integral membrane proteins that participate in the activation of T cells in response to an antigen. The TCR expressed by the majority of T cells consisting of a heterodimer of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. The cytokines can be directly toxic to target cells and can mobilize potent inflammatory mechanisms. CD8+T cells, can develop into cytotoxic T-lymphocytes (CTLs) capable of efficiently lysing target cells that express antigens recognized by the CTLs.

Naïve conventional CD4 T cells can differentiate into four distinct T cell populations, a process that is determined by the pattern of signals they receive during their initial interaction with antigen. These 4 T cell populations are TH1, TH2, TH17, and induced regulatory T (iTreg) cells. THI cells, which are effective inducers of cellular immune responses, mediate immune responses against intracellular pathogens, and are responsible for the induction of some autoimmune diseases. Their principal cytokine products are IFNγ (which enhances several mechanisms important in activating macrophages to increase their microbiocidal activity), lymphotoxin a (LTα), and IL-2, which is important for CD4 T cell memory. TH2 cells, which are effective in helping B cells develop into antibody producing cells, mediate host defense against extracellular parasites, are important in the induction and persistence of asthma and other allergic disease, and produce IL-4, IL-5, IL-9, IL-10 (which suppresses THI cell proliferation and can suppress dendritic cell function), IL-13, IL-25 (signaling through IL-17RB, enhances the production of IL-4, IL-5, and IL-13 by a c-kit-FcERI-nonlymphocyte population, serves as an initiation factor as well as an amplification factor for TH2 responses) and amphiregulin. IL-4 and IL-10 produced by TH2 cells block IFNγ production by THI cells. TH17 cells produce IL-17a, IL-17f, IL-21, and IL-22. IL-17a can induce many inflammatory cytokines, IL6 as well as chemokines such as IL-8 and plays an important role in inducing inflammatory responses. Treg cells play a critical role in maintaining self-tolerance and in regulating immune responses. They exert their suppressive function through several mechanisms, some of which require cell-cell contact. The molecular basis of suppression in some cases is through their production of cytokines, including TGFβ, IL-10, and IL-35. TGFβ produced by T reg cells may also result in the induction if iTreg cells from naïve CD4 T cells. CD4+T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. Zhu, J. and Paul, WE, Blood (2008) 112:1557-69). Resting naïve CD8+ T cells, when primed by antigen presenting cells that have acquired antigens from the infected macrophages through direct infection or cross-presentation in secondary lymphoid organs, such as lymph nodes and spleen, react to pathogens by massive expansion and differentiation into cytotoxic T lymphocyte effector cells that migrate to all corners of the body to clear the infection. In the majority of viral infections, however, CD8 T cell activation requires CD4 effector T cell help to activate dendritic cells for them to become able to stimulate a complete CD8 T cell response. CD4 T cells that recognize related antigens presented by the APC can amplify the activation of naïve CD8 T cells by further activating the APC. B7 expressed by the dendritic cell first activates the CD4 T cells to express IL-2 and CD40 ligand. CD40 ligand binds CD40 on the dendritic cell, delivering an additional signal that increases the expression of B7 and 4-1BBL by the dendritic cell, which in turn provides additional co-stimulation to the naïve CD8 T cell. The IL-2 produced by activated CD4 T cells also acts to promote effector CD T cell differentiation.

The term “TALEN (Transcription Activator-like Effector Nuclease”) refers to a technology that leverages artificial restriction enzymes generated by fusing a TAL effector DNA-binding domain to a DNA cleavage domain for gene editing.

The term “TCR/CD3 complex” as used herein refers to a protein complex composed of four distinct chains. In mammals, the complex contains a CD3γ chain, a CD38 chain, and two CD3& chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

The term “THI cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. THI cells secrete IFN-gamma, IL-2, IL-10, and TNF-alpha/beta. IL-12 and IFN-γ make naïve CD4+T cells highly express T-bet and STAT4 and differentiate to THI cells. (Zhang, Y. et al. Adv. Exp. Med. Bio. (2014) 841:15-44)/

The term “TH2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naïve CD4+T cells highly express STAT6 and GATA3 and differentiate to TH2 cells. (Zhang, Y. et al. Adv. Exp. Med. Bio. (2014) 841:15-44)/

The term “TH17 cells” as used herein refers to a CD4+T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, L A, et al., Immunol. Rev. (2008) 223:87-113]. The key transcription factor in TH17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated Tconv activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/fimmu.2019.01098].

The term “TACI” or “Transmembrane Activator Calcium modulator and cyclophilin ligand Interactor” as used herein refers to a receptor for BAFF expressed on B cells TACI is able to recruit TNF receptor associated factor (TRAF) 2, 5 and 6 to its cytoplasmic domain [Gardam, S. and Brink, R. Frontiers Immunol. 4: article 509 (2014), and has been shown to activate NF-κB1, AP-1 and NFAT signaling pathways. [Id., citing von Bulow, G U and Bram, RJ. Science (1997) 278:138-41]. TRAF2 is an adapter molecule that regulates activation of NF-kappa-B and JNK and plays a central role in the regulation of cell survival and apoptosis; it is required for normal antibody isotype switching from IgM to IgG. TRAF5 is an adaptor protein and signal transducer that links member of the TNFR family to different signaling pathways by association with the receptor cytoplasmic domain and kinases; it mediates activation of NF-κB and probably JNK. TRAF6 is an E3 ligase that produces a K63 polyubiquitin signaling scaffold in TLR-4 signaling to activate the NF-κB pathway. TACI signals activate B cell proliferation, isotype switching and antibody production.

The term “targeted therapy” as used herein refers to a type of cancer treatment that targets proteins that control how cancer cells grow, divide and spread.

The term “TET2” refers to a gene, the protein encoded by which is a methylcytosine dioxygenase that catalyzes the conversion of methylcytosine to 5-hydroxymethylcytosine. It is an epigenetic regulator of cell differentiation and the inflammatory response. [Ferrone, C K et al. Intl J. Mol. Sci. (2020) 21 (2): 626].

The term “therapeutic effect” as used herein is meant to refer to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

The term “therapeutic window” as used herein is meant to refer to a concentration range that provides therapeutic efficacy without unacceptable toxicity. Following administration of a dose of a therapeutic agent/drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

The term “thymus dependent” or T-dependent antigen as used herein refers to antigens that require the cooperation of TH cells for B cell activation and the synthesis of specific antibodies. Presentation of thymus-dependent antigen to T cells must be in the context of MHC class II molecules.

The term “thymus-independent” or “T independent antigen” as used herein refers to antigens that can stimulate B cells without the help of T cells. They tend to be of high molecular weight and are characterized by a repeating epitope structure. The complex polysaccharides present on bacterial surfaces are representative of and perhaps the most studied of the natural T-independent antigens

The term “TIGIT”, refers to a member of the Ig super family and an immune inhibitory receptor.

The term “TIM-3” as used herein refers to a transmembrane protein and immune checkpoint receptor. It is associated with tumor-mediated immune suppression.

The term “tissue-resident memory T cell” or “TRM” as used herein refers to memory lymphocytes that do not migrate after taking up residence in barrier tissues, where they are retained long term. They appear to be specialized for rapid effector function after restimulation with antigen or cytokines at sites of pathogen entry.

The term “tolerance” as used herein refers to the failure to respond to a particular antigen. Tolerance mechanisms that operate in the thymus before the maturation and circulation of T cells are referred to as “central tolerance.” Not all antigens of which T cells need to be tolerant are expressed in the thymus, and therefore central tolerance mechanisms alone are insufficient. Additional tolerance mechanisms exist to restrain the numbers and or function of T cells that are reactive to developmental or food antigens, which are not typically expressed. Tolerance acquired by mature circulating T cells in the peripheral tissues is called “peripheral tolerance.”.

The term “toxicity” as used herein refers to the degree to which a substance can harm humans or animals. Acute toxicity involves harmful effects in an organism through a single or short-term exposure.

The term “TP53” as used herein refers to a gene that provides instructions for making the protein tumor protein p53 (or p53), which acts as a tumor suppressor. In the cell, p53 protein binds DNA, which in turn stimulates another gene to produce a protein called p21 that interacts with a cell division-stimulating protein (cdk2). When p21 is complexed with cdk2 the cell cannot pass through to the next stage of cell division. Mutant p53 can no longer bind DNA in an effective way, and as a consequence the p21 protein is not made available to act as the ‘stop signal’ for cell division.

The term “TRAC” (T cell Receptor Alpha Constant) refers to a gene encoding the constant region of the T cell receptor alpha chain.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (c) limiting recurrence of symptoms in patients that were previously asymptomatic for the disorder(s).

The term “tumor associated antigen” or “TAA” refers to a protein or other molecule that has elevated levels on tumor cells but that is also expressed at lower levels on healthy cells. Tumor specific antigens (“TSA”) are found on cancer cells only.

The term “tumor associated macrophages” or “TAMs” as used herein refers to an immunosuppressive macrophage subtype found in the tumor microenvironment that is involved in the progression and metastasis of cancer. TAMs are broadly considered M2-like, which can be further classified into the M2a phenotype (induced by IL-4 or IL-13), M2b phenotype (IL-10 high, IL-12 low) and M2c phenotype (TNF-α low) according to distinct signal stimuli. They produce abundant growth factors, extracellular matrix (ECM) remodeling molecules and cytokines for the regulation of cancer proliferation via noncoding RNAs, exosomes and epigenetics [Yan, S. and Wan, G. The FEBS Journal (2021) 288 (21): 6174-86, citing Qian, B Z and Pollard, JW. (Cell (2010) 141:39-51]. Activated M2 macrophages distinctively express arginase 1 (ARG1). TAMs can demonstrate direct inhibition on the cytotoxicity of T-lymphocyte through multiple mechanisms and characteristics of tumor evolution, including immune checkpoint engagement via expression, production of inhibitory cytokines [such as IL-10 and transforming growth factor (TGF)-β] and metabolic activities consisting of depletion of 1-arginine (or other metabolites) and the production of reactive oxygen species (ROS). The suppressive immune response renders cancer cells capable of escaping from immune surveillance.

The term “tumor infiltrating lymphocytes” as used herein refers to a heterogeneous lymphocyte population mainly composed of T lymphocytes that may consist of numerous antitumor effector and/or regulatory T cells (Tregs) and are key players in the host's immune response to a tumor. [Wang, J. et al. BMC Cancer (2020) 20:731].

The term “tumor microenvironment” or “TME” refers to the cellular environment in which tumors or cancer stem cells exist.

The term “TME macrophages” as used herein refers to macrophages that arise primarily from bone marrow-derived monocytes that are recruited by tumor or stroma-derived chemokines such as colony-stimulating factor 1 (CSF1; also known as M-CSF) and CCL2. M1 and M2 phenotypes are differentiated in response to different signal stimuli and are polarized according to the TME, exhibiting strong plasticity, such that macrophages adopt context-dependent phenotypes when stimulated [Yan, S. and Wan, G. The FEBS Journal (2021) 288 (21): 6174-86, citing Murry, P J and Wynn, TA. Nat. Rev. Immunol. (2011) 11:723-37]. Antitumorigenic M I macrophages express high levels of tumor necrosis factor (TNF), inducible nitric oxide synthase (INOS; also known as NOS2) and major histocompatibility complex (MHC) class II molecules, whereas pro-tumorigenic M2 macrophages are marked with high levels of arginase 1 (ARG1), interleukin (IL)-10, CD163, CD204 or CD206 expression. The activation of primary macrophages into MI or M2 phenotype is mainly induced by interferon-regulatory factor/signal transducer and activator of transcription signaling pathways [[Id., citing Waqas, S F H et al. in Nuclear Receptors: Methods and Experimental Protocols, MZ Badr. Ed., Springer, New York, NY, pp. 211-24].

The term “whole blood” as used herein refers to generally unprocessed or unmodified blood collected from a subject containing all of its components, including, but are not limited to, plasma, cellular components (e.g., red blood cells, white blood cells (including lymphocytes, monocytes, cosinophils, basophils, and neutrophils), and platelets), proteins (e.g., fibrinogen, albumin, immunoglobulins), hormones, coagulation factors, and fibrinolytic factors. The term “whole blood” is inclusive of any anticoagulant that may be combined with the blood upon collection.

### EMBODIMENTS

The present disclosure provides B cell maturation antigen (BCMA) as a specific target for post-consolidation immunotherapy for eligible patient populations having AML with high-risk disease.

According to one aspect, the present disclosure provides a method for treating an eligible subject with acute myeloid leukemia including high-risk disease features comprising administering to the subject post-consolidation a targeted immunotherapy comprising an immunotherapeutic agent specifically targeting B cell maturation antigen.

According to some embodiments, the eligible subject with AML is a child. According to some embodiments the child is less than 2 years of age. According to some embodiments, the child is teenage, e.g., 13-19 years old, inclusive. According to some embodiments, the eligible subject with AML is an adult. According to some embodiments, the eligible subject is a young adult, aged 26-46, inclusive. According to some embodiments, the eligible subject is 60-69 years of age, inclusive. According to some embodiments, the eligible subject is 70 or older.

According to some embodiments, high risk features include biologic features, clinical features, or both.

According to some embodiments, high risk biological features include an antecedent hematological disorder; presence of ≥20% BM blasts in the bone marrow, a clonal cytogenetic abnormality, and a molecular abnormality.

According to some embodiments, the antecedent hematological disorder is myelodysplastic syndrome. According to some embodiments, the antecedent hematological disorder is refractory AML. According to some embodiments, the antecedent hematological disorder is AML in remission. According to some embodiments, the antecedent hematological disorder is mixed phenotype acute leukemia (previously referred to as bilineage leukemia and biphenotypic acute leukemia).

Clinical variables include comorbidities, measurable residual disease at time of complete remission, AML refractory to induction chemotherapy; remission after induction therapy, a relapsed subject in remission, AML arising out of an antecedent hematologic disorder; and AML in the elderly.

According to some embodiments, the high risk molecular feature is an FLT3 mutation, an NPMI mutation; an isocitrate dehydrogenase 1 or 2 (IDH1/2) mutation, a RUNX1 mutation, a DNMT3A mutation, a TET2 mutation, a TP53 mutation, or a combination thereof. According to some embodiments, the FLT3 mutation is an FLT3-ILD mutation.

According to some embodiments, high risk cytogenetic features include chromosomal translocations (e.g., t (8;21) (q22;q22.1); RUNX1-RUNXIT1); and monosomies of any chromosome (e.g., monosomy chromosome 5, monosomy chromosome 7, or both).

According to some embodiments, high risk features in the elderly include one or more of a high incidence of cytogenetic abnormalities involving monosomies 5 and 7 and chromosome 17 abnormalities, a high incidence of multiple mutations including TP53, and a high incidence of secondary/therapy-related AML; and co-morbidities (e.g., hypertension; diabetes; organ dysfunctions including cardiac, pulmonary and renal abnormalities).

According to some embodiments, the immunotherapy includes at least one of the following.
(1) According to some embodiments, AML blasts can be targeted with a BCMA targeted antibody drug conjugate (ADC). According to some embodiments, the ADC is described in Table 1.
(2) Immune checkpoint therapy, which helps cancer-fighting T cells mount a longer-lasting response. Immune checkpoints engage when proteins on the surface of T cells recognize and bind to partner immune checkpoint proteins on other cells, such as some tumor cells. When the checkpoint and partner proteins bind together, they send an “off” signal to the T cells, which can prevent the immune system from destroying the cancer. Immunotherapy drugs called immune checkpoint inhibitors block checkpoint proteins from binding with their partner proteins. This prevents the “off” signal from being sent, allowing the T cells to kill cancer cells.

Examples of checkpoint inhibitors include anti-PDI (e.g., lambrolizumab/pembrolizumab (KEYTRUDA®) and nivolumab (OPDIVO®), anti-PD-L1 (Atezolizumab, TECENTRIQ®, MPDL3280A), and anti-CTLA4 (ipilimumab, YERVOY®).

Since upregulation of inhibitory immune checkpoints by tumor and TME cells represents a mechanism of resistance to ICEs, according to some embodiments, any of the following immunotherapies can be combined with a checkpoint inhibitor to overcome immune escape by BCMA-expressing AML blasts.

(3) Immune cell engagers” (or “ICEs”) are molecules able to redirect immune effector cells (regardless of their antigen specificity) against cancer cells for major histocompatibility complex (MHC)-independent cancer cell elimination and generation of immune responses against poorly immunogenic tumors with the aim of triggering an efficient tumor cell killing. [Fuca, G. et al. Immune cell engagers in solid tumors: promises and challenges of the next generation immunotherapy. ESMO Open (2021) 6 (1): 100046, citing Brischwein, K. et al. MT110: a novel bispecific single-chain antibody construct with high efficacy in eradicating established tumors. Mol. Immunol. (2006) 43 (8): 1129-43]. Most ICEs are trans-binding bispecific antibodies (bsAbs) usually consisting of two linked single-chain fragment variables (scFvs) that originate from different monoclonal antibodies: one scFv recognizes a surface TAA, whereas the other is specific for a certain membrane molecule expressed on effector immune cells. [Id., citing Choi, B D et al. Bispecific antibodies engage T cells for antitumor immunotherapy. Expert Opin. Biol. Ther. (2011) 11 (7): 843-53] The scFv with specificity for the effector immune cell must be also able to trigger an appropriate signal transduction cascade to activate the killing machinery. The compact structure resulting from the link of these two different scFvs allows the formation of the immune synapsis between tumor and immune cells and eventually leads to tumor cell elimination. In order to avoid the risk of an uncontrolled triggering and subsequent toxicity, the activation of effector cells takes place only when both bsAbs ‘arms’ are engaged with their respective target antigens. [Id., citing Bargou, R. et al. Tumor regression in cancer patients by very low doses of a T cell-engaging antibody. Science (2008) 321 (5891): 974-77].

The killing of nearby target-negative cells via the release of molecules that diffuse locally upon ICEs-mediated effector cell activation in the presence of target-positive cells (“bystander killing” effect) contributes to the antitumor activity of ICEs. Recent insights suggest that the diffusion of released cytokines leads to the upregulation of cell surface molecules (e.g. ICAM-1 and FAS) on bystander cells. The expression of these molecules makes bystander cells susceptible to effector cell-mediated killing even in the absence of a regular cytolytic synapse. Id., citing Ross, S L et al. Bispecific T cell engager (BiTE® antibody constructs can mediate bystander tumor cell killing. PLOS One (2017) 12 (8): e0183390). This phenomenon is of particular interest in solid tumors that are characterized by a marked heterogeneity of TAA expression and by an immune suppressive tumor microenvironment (TME): a bystander killing effect can mitigate antigen escape and contribute to eliminating the pro-tumoral cellular compartment of TME.

ICEs developed to date can be divided into three main categories: T cell, natural killer (NK) cell and cytotoxic/phagocytic cell engagers.

### (a) T Cell Engagers

Bispecific T cell engagers (BiTes), the most common class of ICEs, consist of a TAA-targeting scFv linked with an scFv usually activating a specific chain of the CD3 complex (mainly the CD3& chain) that is associated with the T cell receptor (TCR) complex and participate in TCR-mediated signaling. [Id., citing Zhu, M. et al. Blinatumomab, a bispecific T-cell engager (BITE®) for CD-19 targeted cancer immunotherapy: clinical pharmacology and its implications. Clin. Pharmacokinet. (2016) 55 (10): 1271-88]. By this approach, T cells are physically redirected against tumor cells and at the same time activated. The formation of this ‘artificial’ immunological synapse is accompanied by the redistribution of signaling and secretory granule proteins in T cells, leading to the release of perforin and granzyme. [Id., citing Offner, S. et al. Induction of regular cytolytic T cell synapses by bispecific single-chain antibody constructs on MHC class I-negative tumor cells. Molec. Immunol. (2006) 43 (6): 763-71]. Such contact-dependent cytotoxicity is likely the main mechanism for BiTes-induced direct killing of tumor cells, as EDTA chelation of Ca2+ (required for perforin multimerization and pore formation) leads to the complete inhibition of target cell apoptosis. [Id., citing Haas, C. et al. Mode of cytotoxic action of T cell engaging BiTE antibody MT110. Immunobiol. (2009) 214 (6): 441-53]. Activation of T cells also results in the secretion of cytokines and T-cell proliferation, which may be required to sustain a durable antitumor immune response. [Id., citing Nguyen, H H et al. Naïve CD8 (+) T cell derived tumor-specific cytotoxic effectors as a potential remedy for overcoming TGF-β immunosuppression in the tumor microenvironment. Sci Rep. (2016) 6:28208]. Together with canonical cytotoxic T cells (CD8+T cells), CD4+T cells, γδ T cells and NK T cells (NKT cells) also can be activated by and contribute to the antitumor activity of BiTEcs specific for the CD3 complex. [Id., citing Kischel, R. et al. Abstract #3252: effector memory T cells make a major contribution to redirected target cell lysis by T cell-engaging BiTE antibody MT110; Quintarelli, C. et al. Choice of costimulatory domains and of cytokines determined CAR T-cell activity in neuroblastoma. Oncoimmunology (2018) 7 (6): e1433528; Perez-Ruiz, E. et al. Anti-CD137 and PD-1/PD-L1 antibodies en route toward clinical synergy. Clin. Cancer Res. (2017) 23 (18): 5326-8]. A co-stimulation molecule (e.g. CD28 or 4-1BB) can also be exploited as a target to engage activated T cells, as showed using a trispecific antibody engaging CD3 and CD28 on T cells, [Id., citing Wu. L. et al. Trispecific antibodies enhance the therapeutic efficacy of tumor-directed T cells through T cell receptor co-stimulation. Nat. Cancer 1 (1) (2020): 86-98] or 4-1BB engaging molecules. [Id., citing Hinner, M J et al. Tumor-localized costimulatory T-cell engagement by the 4-1BB/HER2 bispecific antibody-anticalin fusion PRS-343. Clin. Cancer Res. (2019) 25 (19): 5878-89; Claus, C. et al. Tumor-targeted 4-1BB agonists for combination with T cell bispecific antibodies as off-the-shelf therapy. Sci. Transl. Med. (2019) 11 (496): caav5989].

According to some embodiments, T cells can be activated and physically redirected against BCMA-expressing AML blasts by a BiTE that targets CD3 on T cells and BCMA expressed on the surface of relapsed/refractory myeloma cells with manageable toxicities, e.g., teclistamab (formerly JNJ-7957, Janssen Biotech, Inc.), [Granger, K. et al. Oncol. Pharm. Pract. (2023) doi: 10.1177/10781552231154809], elranatamab (Pfizer), AMG-701, AMG 420 (formerly BI 836909, Amgen) [Topp, M S et al. J. Clinical Oncol. (2020) 38 (8): 775-83, citing Hipp, S. et al. Leukemia (2017) 31:1743-51; Cohen, AD. Hematology Am. Soc. Hematol. Educ. Program (2019) 2019 (1): 266-72]; REGN5458 (Regeneron)., TNB-383B [Acquired by Abbvie from TencoOne, Inc.; Rodriguez, C. et al. Blood (2020) 136 (Suppl. 1): 43-44) . . .

According to some embodiments, the bispecific antibody directed against CD3 on T cells and BCMA expressed on the surface of AML blasts can be of any design format, e.g., tandem scFv molecules; a bivalent diabody format; a single chain scDb format; dimeric tetravalent derivatives thereof, ‘tandAb’ molecules with two binding sites for each antigen, disulfide-stabilized variants, such as a dual affinity retargeting molecules (DART), fusion of antigen binding moieties to other protein domains (e.g., albumin) or a molecule created by a dock-and-lock approach whereby domains created by heterodimerization of a protein can be linked to Fab fragments to form multivalent bsAb.

According to some embodiments, the toxicity profile of BCMA targeted therapies in MM patients may include depletion of some B cell subsets and plasma cells and hypogammaglobulinemia, which is associated with an increased risk for infections. According to some embodiments, cytokine release syndrome, the most common adverse event of BCMA targeted T-cell engager therapy can be adequately managed, e.g., by administration of tocilizumab or steroids.

According to some embodiments, the T cell-directing bispecific antibody may be combined with a checkpoint inhibitor. Examples of checkpoint inhibitors include anti-PDI (e.g., lambrolizumab/pembrolizumab (KEYTRUDA®) and nivolumab (OPDIVO®), anti-PD-L1 (Atezolizumab, TECENTRIQ®, MPDL3280A), and anti-CTLA4 (ipilimumab, YERVOY®).

According to some embodiments, the T cell-directing bispecific antibody may be combined with a gamma-secretase inhibitor (GSI) in order to overcome shedding of membrane-bound BCMA. Examples include, without limitation, LY3039478/JSMD194, dihydroergocristine (DHEC, Lei, X. et al. Sci. Repts. (2015 5:16541, RO4929097 (NCT01070927); LY900009 (NCT01158404); MK-0752 (NCT00756717); PF-03084014 (NCT01981551); BMS-986115 (NCT01986218); GSI-136 (NCT00719394); AL-101 (NCT04461600); and Nirogacestat (NCT05041036).

According to some embodiments, the T cell-directing bispecific antibody may be combined with both a checkpoint inhibitor and a gamma secretase inhibitor.

### (b) NK Cell Engagers

NK cells are characterized by the lack of TCR and CD3 molecules and by the expression of CD56 (also known as neural cell adhesion molecule) and CD16 (also known as FcγRIII). [Id., citing Shimasaki, N. et al. nat. Rev. Drug Discov. (2020) 19 (3): 200-18]. NK cells activity is balanced by specific membrane receptors with activating (e.g. natural cytotoxicity receptors, like CD16) or inhibitory (e.g. inhibitory killer immunoglobulin-like receptors) functions. [Id., citing Lanier, LL. Up on the tightrope: natural killer cell activation and inhibition. Nat. Immunol. (2008) 9 (5): 495-502]. CD16 currently is the most implemented NK cell target for the development of ICEs in the format of bsAbs. Data from preclinical studies has shown that CD16-directed ICEs are able to activate NK cells and induce TAA-specific cytotoxicity with cytokine and chemokine production. Id., citing Gleason, M K et al. Bispecific and trispecific killer cell engagers directly activate human NK cells through CD16 signaling and induce cytotoxicity and cytokine production. Mol. Cancer Ther. (2012) 11 (12): 2674-84]. CD16-directed ICEs have shown antitumor activity in hematological malignancies and AFM13 (a CD30×CD 16 bispecific compound) is in phase II clinical development for the treatment of Hodgkin's lymphoma [Id., citing Rothe, A. et al. A phase I study of the bispecific anti-CD30/CD16A antibody construct FM13 in patients with relapsed or refractory Hodgkin lymphoma. Blood (2015) 125 (26): 4024-31]. In solid tumors, CD16-directed ICEs have preclinically been shown to induce effective responses in several solid tumor models. [Id., citing Godbersen, C. et al. NKG2D ligand-targeted bispecific T-cell engagers lead to robust antitumor activity against diverse human tumors. Mol. Cancer Ther. (2017) 16 (7): 1335-46; Rothe, A. et al. The bispecific immunolig and ULBP2-aCEA redirects natural killer cells tumor cells and reveals potent anti-tumor activity against colon carcinoma. Int. J. Cancer (2014) 134 (12): 2829-40]. Another approach to exploit NK cytotoxicity using ICEs involves the engagement of the activating NKG2D receptor. In preclinical models, NKG2D-directed bsAbs demonstrated activity both in vitro and in vivo against carcinoembryonic antigen (CEA)- and human epidermal growth factor receptor 2 (HER2)-positive tumors, [Id., citing Godbersen, C. et al. NKG2D ligand-targeted bispecific T-cell engagers lead to robust antitumor activity against diverse human tumors. Mol. Cancer Ther. (2017) 16 (7): 1335-46] and a CD24×NKG2D bsAb demonstrated in vivo activity in a model of hepatocellular carcinoma in combination with sorafenib. [Id., citing Han, Y. et al. CD24 targeting bi-specific antibody that simultaneously stimulants NKG2D enhances the efficacy of cancer immunotherapy. J. Cancer Res. Clin. Oncol. (2019) 145 (5): 1179-90].

According to some embodiments, NK cells can be activated and physically redirected against BCMA-expressing AML blasts by a bispecific NK cell engager targeting CD16 or an NKG2D receptor.

According to some embodiments, the NK cell-directing bispecific antibody may be combined with a checkpoint inhibitor. Examples of checkpoint inhibitors include anti-PD1 (e.g., lambrolizumab/pembrolizumab (KEYTRUDA®) and nivolumab (OPDIVO®), anti-PD-L1 (Atezolizumab, TECENTRIQ®, MPDL3280A), and anti-CTLA4 (ipilimumab, YERVOY®).

According to some embodiments, the T cell-directing bispecific antibody may be combined with a gamma-secretase inhibitor (GSI) in order to overcome shedding of membrane-bound BCMA. Examples include, without limitation, LY3039478/JSMD194, dihydroergocristine (DHEC, Lei, X. et al. Sci. Repts. (2015 5:16541, RO4929097 (NCT01070927); LY900009 (NCT01158404); MK-0752 (NCT00756717); PF-03084014 (NCT01981551); BMS-986115 (NCT01986218); GSI-136 (NCT00719394); AL-101 (NCT04461600); and Nirogacestat (NCT05041036).

According to some embodiments, the NK cell-directing bispecific antibody may be combined with both a checkpoint inhibitor and a gamma secretase inhibitor.

### (c) Cytotoxic/Phagocytic Cell Engagers

Cytotoxic/phagocytic immune cells (i.e. monocytes, macrophages, dendritic cells and cytokine-activated neutrophils) can be engaged via the non-ligand binding site of the high-affinity receptor for immunoglobulin G (FcγRI, also known as CD64) which is selectively expressed by these immune cells. [Id., citing Schwaab, T. et al. Phase I pilot trial of the bispecific antibody MDXH210 (anti-Fc gamma R1 X anti-Her-2/neu) in patients whose prostate cancer overexpresses HER-2/neu. J. Immunother. (2001) 24 (1): 79-87]. Chemically linked bispecific molecules engaging CD64 and targeting a TAA are able to trigger antibody-dependent cell-mediated cytotoxicity and cytotoxic lysis of tumor cells as shown in preclinical models of solid tumors targeting HER2 and epithelial cell adhesion molecule (EpCAM). [Id., citing Schweizer, C. et al. Efficient carcinoma cell killing by activated polymorphonuclear neutropils targeted with an Ep-CAMxCD64 (HEA125×197) bispecific antibody. Cancer Immunol. Immunother. (2002) 51 (11-12): 621-9].

According to some embodiments, cytotoxic/phagocytic immune cells can be activated and physically redirected against BCMA-expressing AML blasts by a bispecific cytotoxic/phagocytic cell engager targeting CD64.

According to some embodiments, the cytotoxic/phagocytic immune cell-directing bispecific antibody may be combined with a checkpoint inhibitor. Examples of checkpoint inhibitors include anti-PD1 (e.g., lambrolizumab/pembrolizumab (KEYTRUDA®) and nivolumab (OPDIVO®), anti-PD-L1 (Atezolizumab, TECENTRIQ®, MPDL3280A), and anti-CTLA4 (ipilimumab, YERVOY®).

According to some embodiments, the T cell-directing bispecific antibody may be combined with a gamma-secretase inhibitor (GSI) in order to overcome shedding of membrane-bound BCMA. Examples include, without limitation, LY3039478/JSMD194, dihydroergocristine (DHEC, Lei, X. et al. Sci. Repts. (2015) 5:16541), RO4929097 (NCT01070927); LY900009 (NCT01158404); MK-0752 (NCT00756717); PF-03084014 (NCT01981551); BMS-986115 (NCT01986218); GSI-136 (NCT00719394); AL-101 (NCT04461600); and Nirogacestat (NCT05041036).

According to some embodiments, the cytotoxic/phagocytic immune cell-directing bispecific antibody may be combined with both a checkpoint inhibitor and a gamma secretase inhibitor.

(4) The term “adoptive cellular therapy” refers to an immunotherapy that modifies a patient's own immune cells to increases the anti-tumor effectiveness of these cells, expands them in vitro and reinfuses them to the patient, which improves the power of the immune response against the cancer. There are four main types of adoptive of adoptive cellular therapy:

(i) a Chimeric Antigen Receptor (CAR) T cell therapy-T cells are genetically engineered to express a molecule known as a chimeric antigen receptor, or CAR and expanded in vitro and then reinfused to the patient to find and fight the cancer.

The U.S. FDA has approved six CAR-T cell products for the treatment of relapsed/refractory B cell malignancies including tisagenlecleucel (KYMERiah®, Novartis), axicabtaghene ciloleucel (YESCARTA®, Kite Pharma, Inc.; brexucabtagene autoleucel (TECARTUS®; Gilead); lisocabtagene maralcucel (BREYANZI®, Bristol Myers Squibb), idecabtagene vicleucel (ABECMA®, Bristol Myers Squibb and Bluebird Bio) and ciltacabtagenc autoleucel (CARVYKTI®; Legend and Janssen).

Each of KYMRIAH, YESCARTA, BREYANZI and TECARTUS are CD19-directed CAR-T therapies. KYMRIAH is a CD19-directed genetically modified autologous T cell immunotherapy indicated for the treatment of adult patients with relapsed or refractory follicular lymphoma after two or more lines of therapy. YESCARTA is an autologous anti-CD19 chimeric antigen receptor T cell therapy indicated for the treatment of adult patients with large B cell lymphoma that is refractory to first-line chemoimmunotherapy or that relapses within 12 months of first line chemoimmnotherapy. BREYANZI is a CD19-directed genetically modified autologous T cell immunotherapy indicated for adult patients with large B cell lymphoma who have refractory disease to first-line chemoimmunotherapy or relapse within 12 months of first-line chemoimmunotherapy; or refractory disease to first line chemoimmunotherapy or relapse after firs-line chemoimmunotherapy and are not eligible for HSCT due to comorbidities or age. TECARTUS is a CD19-directed genetically modified autologous T cell immunotherapy indicated for adult patients with relapsed or refractory mantle cell lympho9ma and for adult patients with relapsed or refractory B-cell precursor acute lymphoblastic leukemia (ALL).

Each of ABECMA and CARVYKYI is a BCMA-directed genetically modified autologous CAR-T cell therapy indicated for the treatment of adult patients with relapsed or refractory multiple myeloma after four or more prior lines of therapy, including an immunomodulatory agent, a proteasome inhibitor, and an anti-CD38 monoclonal antibody.

According to some embodiments, AML blasts can be targeted with a BCMA-targeted CAR-T therapy. According to some embodiments, the CAR-T therapy is described in Table 1.

According to some embodiments, the CAR structure consists of an extracellular antigen-recognition domain, a transmembrane domain and an intracellular signaling domain. The extracellular domain, a single-chain variable fragment (scFv) is able to specifically recognize tumor surface antigens, e.g., a tumor-associated antigen (TAAs) or tumor specific antigen (TSA). Once the tumor antigen is identified by scFV, CAR-Ts are activated and transmit activation signals to the intracellular domain.

According to some embodiments, first generation CAR-constructs contain an scFv and an intracellular CD33 activation domain. According to some embodiments, second generation CAR constructs add a costimulatory domain, such as CD28, 4-1BB, OX40 or ICOS, which increases their proliferative capacity and ability to release cytokines. According to some embodiments, third generation cAR constructs encompass two co-stimulatory molecules, such as CD28 and 4-1BB. According to some embodiments, fourth generation CAR constructs, also referred to as armored CARs, is additionally modified to secrete cytokines such as IL-7, IL-12, IL-15, IL-21 or to express safety switch suicide genes to regulate the persistence or function of CAR-T cells, such as iCaspase-9.

An APRIL-based CAR-T cell therapy has been described for dual targeting of BCMA and TACI in multiple myeloma. [Lee, L. et al. Blood (2018) 131 (7): 746-58]. According to some embodiments, the AML may be treated with an APRIL-based CAR-T cell therapy. [Lec, L. et al. Blood (2018) 131:746-58]. According to some embodiments, a trimeric APRIL-based CAR comprising a trimer composed of 3 April monomers connected by linkers so each CAR has three binding domains with only one co-stimulatory molecule 4-1BB that can eradicate MM cells that do not express BCMA in vivo, which relapses due to targeted escape., may be used to treat the AML (Schmidts, A. et al. Blood Advances (2019) 3:3248-60) may treat AML.

(ii) a Chimeric Antigen Receptor (CAR) natural killer (NK) cell therapy. NK cells are immune system cells that identify and then kill abnormal cells, including some cancer cells. Many cancers are good at avoiding detection, which limits the ability of NK cells to fight the disease naturally. In CAR NK cell therapy, NK cells are engineered to express a molecule known as a chimeric antigen receptor, or CAR to better recognize cancer, boosting their ability to find and kill cancer cells.

(iii) a tumor infiltrating lymphocyte (TIL) therapy, which uses a patient's T cells that are collected from a piece of surgically-removed tumor. While these cells may recognize the cancer, there are too few of them to succeed. The number of these cells is increased substantially in the lab and then given back to the patient.

(iv) an endogenous T cell (ETC) therapy, which uses T cells from a patient's blood. From this diverse pool of T cells, doctors select only those that may recognize signatures specific to the cancer. The number of these specific T cells is increased substantially and then given back to the patient.

According to some embodiments, the effective dose of the immunotherapy agent destroys tumor cells through direct lysis or by effecting destruction of the tumor cells indirectly, e.g., by mobilizing attracting cell cytotoxicity agents through secretion of cytokines.

According to some embodiments, the effective dose of the immunotherapy agent mobilizes the patient's immune response to the tumor cells, where the term “mobilizes” as used herein means to put into motion or use, become ready or capable of being moved quickly and with relative ease. stimulates activation of the patient's lymphocyte populations.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Mean Fluorescence Intensity Experiments In Vitro

Cell lines and patient cells. The human MM cell lines MM.1S, U266 and NCI-H929 and the human AML cell lines THP-1, OCI-AML3, MOLM-13, KO52, KASUMI-1, SET-2, NKM-1, HL-60 and HEL were maintained and propagated in RPMI 1640 medium supplemented with 10% heat-inactivated fetal bovine serum (GIBCO BRL, Gaithersburg, MD), 1% L-glutamine, and penicillin-streptomycin in a humid environment of 5% CO₂ at 37° C.

Peripheral blood or bone marrow mononuclear cell samples from AML patient cells were obtained from the Fred Hutchinson/University of Washington Hematopoietic Diseases Repository.

U266, MOLM-13 and OCI-AML3 cells were transduced using 3^{rd} generation lentiviral vector with bi-cistronic expression of dtomato and firefly luciferase (pUltra-Chili-Luc) (Addgene #48688). Transduced cells were expanded and individual clones/populations with high dTomato expression were sorted using a BD FACSMelody™cell sorter. Bioluminescence signal intensity of single clones was determined, and the brightest clones were used for killing assays.

Antibodies and flow cytometry. Cells were stained with anti-human CD269 (BCMA) monoclonal antibody (clone 19F2, BioLegend, San Diego, CA), anti-human CD33 (clone hP67.6, Absolute Antibody, Boston MA), anti-human CD123 (clone SSDCLY107D2, Beckman Coulter) and antihuman CD135 (Flt3) (clone BV10A4H2, Biolegend) for 15 minutes at 4° C. in phosphate buffered saline (PBS) with 0.5% bovine serum albumin (BSA) (PBS/BSA), fixed in 2% paraformaldehyde for 15 minutes, washed, and resuspended in PBS/BSA prior to analysis on BD LSRFortessa™ Flow Cytometer (Becton Dickinson (BD), Franklin Lakes, NJ). FCS files were and analyzed with FlowJo (version 10.5.3) software (BD) by gating on viable AML blasts and generating quantitative fluorescence intensity measurements (median fluorescence intensity (MFI)) resulting from antibody binding to surface molecules on AML cells. The mean and standard error of the mean (SEM) of BCMA, CD33, CD123 and Flt3 MFI are presented.

Killing Assays. BCMA-expressing firefly luciferase-transduced AML target cells were cocultured with healthy volunteer-derived PBMCs in the presence or absence of an anti-BCMA-anti-CD3 bispecific antibody (BPS Bioscience, San Diego, CA). Target cell numbers after 24 to 72 hours of co-culture were quantified indirectly by luciferase assay using a multimodal microplate reader. The mean and SEM of normalized relative luminescence units (RLU) are presented. A luminometer detects the photon emission produced from the luciferase reaction and the unit of measurement comes out as relative light units (RLU).

### Results

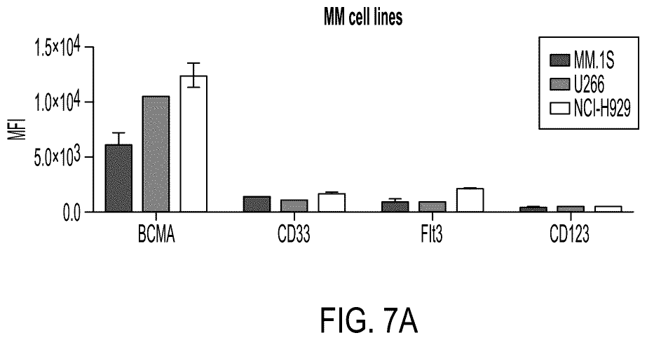

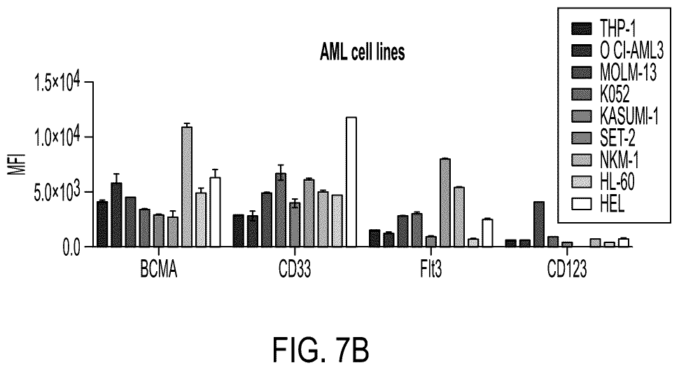

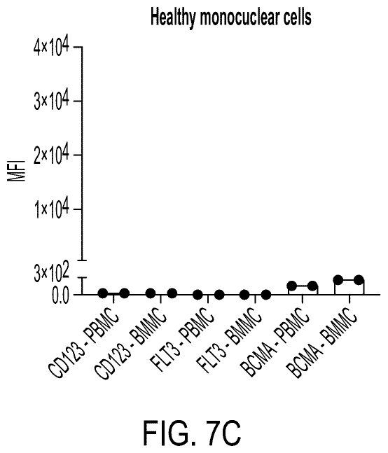

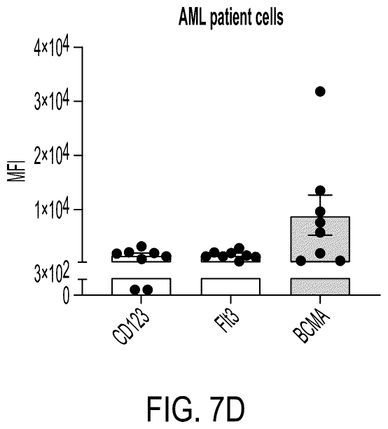

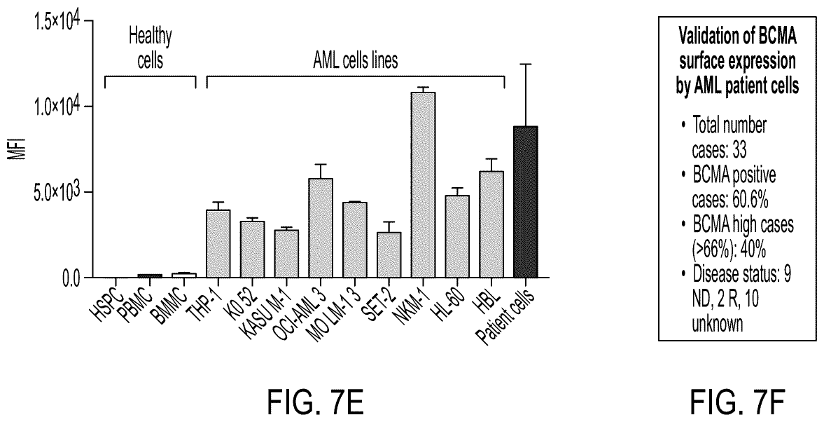

FIG. 7A, FIG. 7B, FIG. 7C, FIG. 7D, FIG. 7E and FIG. 7F provides mean fluorescence intensity data showing that BCMA is highly expressed in AML cell lines and patient cells.

FIG. 7A. Three human MM cell lines (MM IS, U266, NCI-H929 were analyzed for the expression of the MM-associated antigen BCMA and the AML-associated antigens CD33, Flt3, and CD123. Mean and SEM of median fluorescence intensities (MFI) are presented.

FIG. 7B. Nine human AML cell lines (THP-1, OCI-AML-3, MOLM-13, K052, KASUMI-1, SET-2, NKM-1, HL-60, HEL) were analysed for expression of the MM-associated antigen BCMA and the AML-associated antigens CD33, Flt3, and CD123. Mean and SEM of MFI are presented.

FIG. 7C. Healthy human peripheral blood mononuclear cells (PBMCs) and bone marrow mononuclear cells (BMMC) were analysed for the expression of BCMA, Flt3, and CD123. Mean and SEM of MFI are presented.

FIG. 7D. AML patient cells were analyzed for the expression of BCMA, Flt3, and CD123. Mean and SEM of MFI are presented.

FIG. 7E. Healthy hematopoietic cells (CD34+HSPC, PBMC, BMMC, white columns) as well as AML cell lines (gray columns) and AML patient cells (black column) were analyzed for the expression of BCMA. Mean and SEM of MFI are presented. FIG. 7F. Summary of BCMA positivity (frequency of BCMA positive cells) of AML cells from 33 patients. ND: newly diagnosed; R: relapsed.

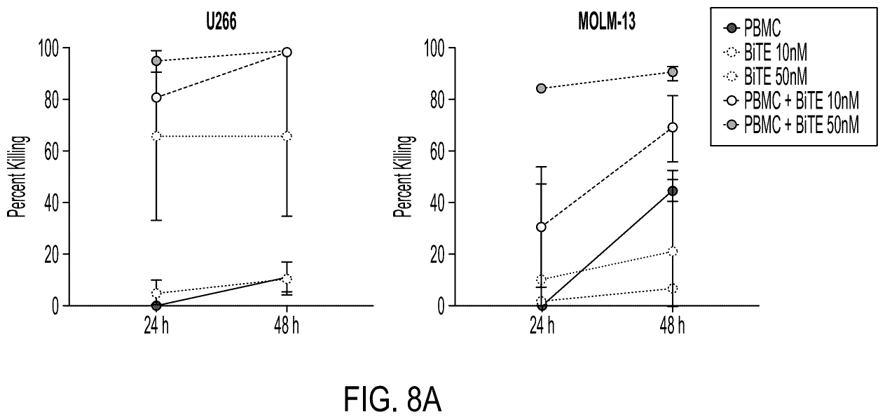

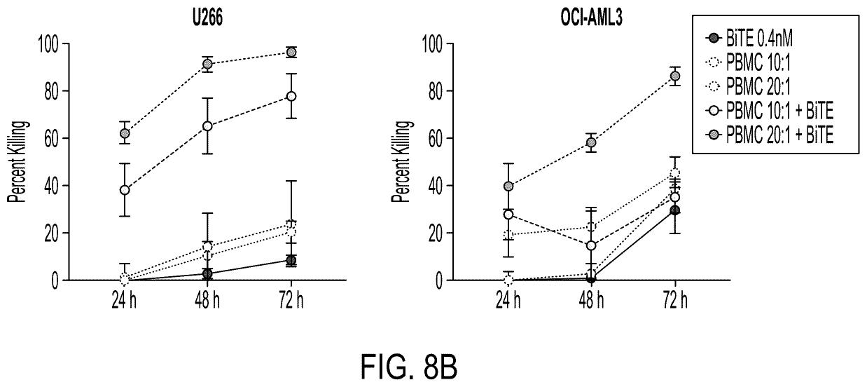

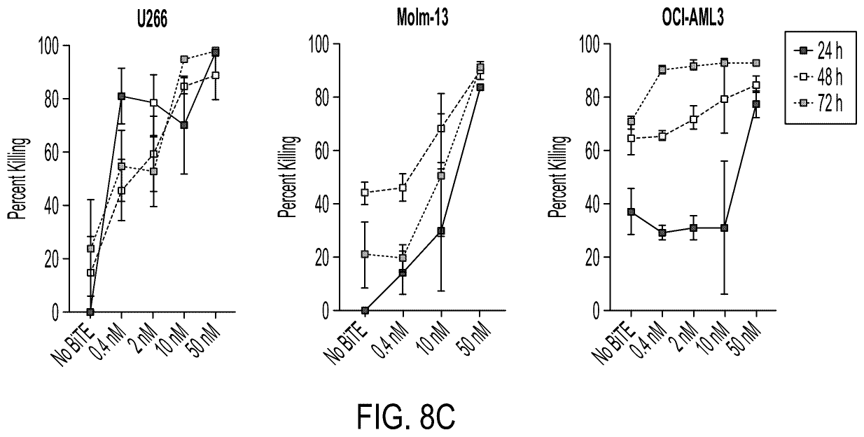

FIG. 8A, FIG. 8B, and FIG. 8C show that while U266 cells were more sensitive to BCMA BiTE mediated killing than AML cells, BCMA BiTE treatment mediates killing of AML cells in vitro.

FIG. 8A is a plot of percent killing vs. time for U266-luc human multiple myeloma cells (left) and MOLM-13-luc human AML cells (right) cultured in the presence of human peripheral blood mononuclear cells (PBMC) at an effector: target ratio of 20:1. BCMA×CD3 BiTE (10 or 50 nM) was added to the media and relative luminescence units (RLU) were measured after 24 and 48 h. Controls included the following conditions: untreated target cells, target cells+PBMC, target cells+BITE. Normalized values (percentage of untreated target cells) are presented (Mean and SEM).

FIG. 8B is a plot of percent killing vs. time for U266-luc human multiple myeloma cells (left) and OCI-AML3-luc human AML cells (right) cultured in the presence of human PBMC at effector: target ratios of 10:1 and 20:1. BCMA×CD3 BITE (0.4 nM) was added to the media and RLU were measured after 24, 48 and 72 h. Controls included the following conditions: untreated target cells, target cells+PBMC, target cells+BiTE. Normalized values (percentage of untreated target cells) are presented (Mean and SEM).

FIG. 8C is a plot of percent killing of U266-luc cells (left), MOLM-13-luc cells (middle) and OCI-AML3 cells (right) cultured in the presence of human PBMC at effector: target ratios of 10:1 (U266), 20:1 (MOLM-13) and 30:1 (OCI-AML3). BCMA×CD3 BiTE was added to the media at the indicated concentrations and RLU were measured after 24, 48 and 72 h. Controls included the following conditions: untreated target cells and target cells+PBMC. Normalized values (percentage of untreated target cells) are presented (Mean and SEM).

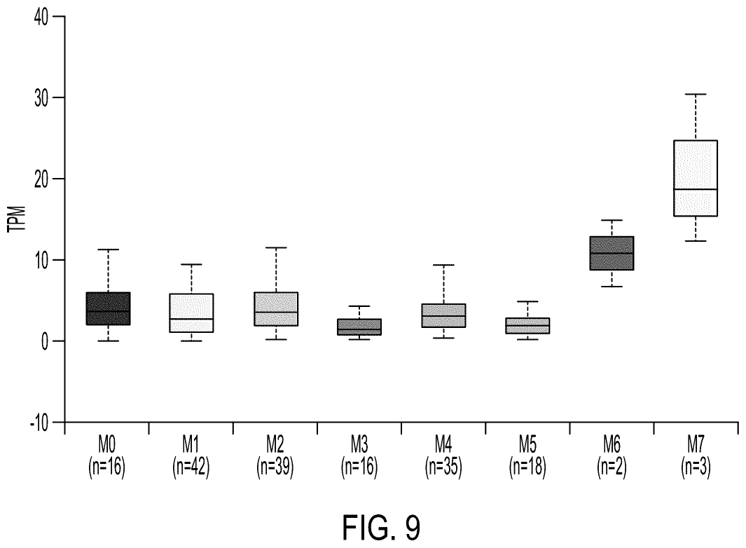

FIG. 9 shows that the BCMA gene (TNFRSF17) is expressed by all AML subsets. Median normalized expression (transcripts per million of BCMA by AML cells cells (transcripts permillion) based on the French-American-British (FAB) classification. The figure was created with the University of Alabama at Birmingham Cancer data analysis Portal (UALCAN) platform using TCGA bulk RNA sequencing gene expression data sets.

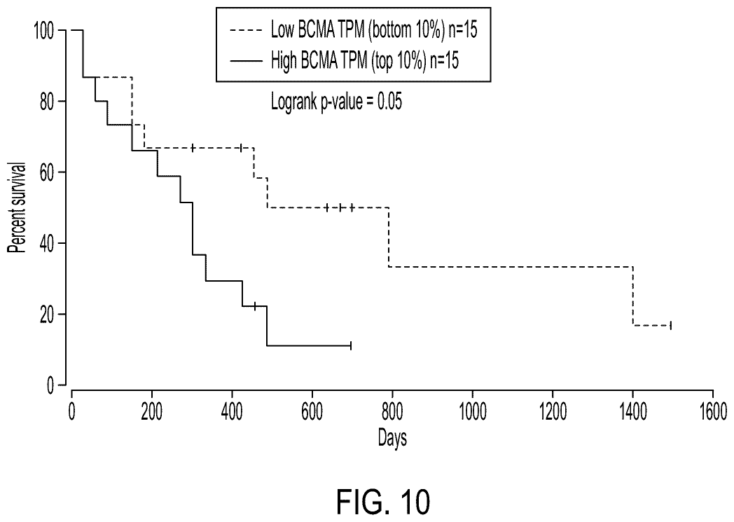

FIG. 10 provides a Kaplan Meyer analysis of AML probability of survival comparing patients with high (top 10%) and low (bottom 10%) BCMA gene normalized expression levels (transcripts per million, TPM). The difference between the groups was analyzed by Log-rank test. The figure was created with the OncoInc platform using The Cancer Genome Atlast program (TCGA) survival data. It shows that shows that high BCMA expression levels are associated with a poor prognosis in AML patients.

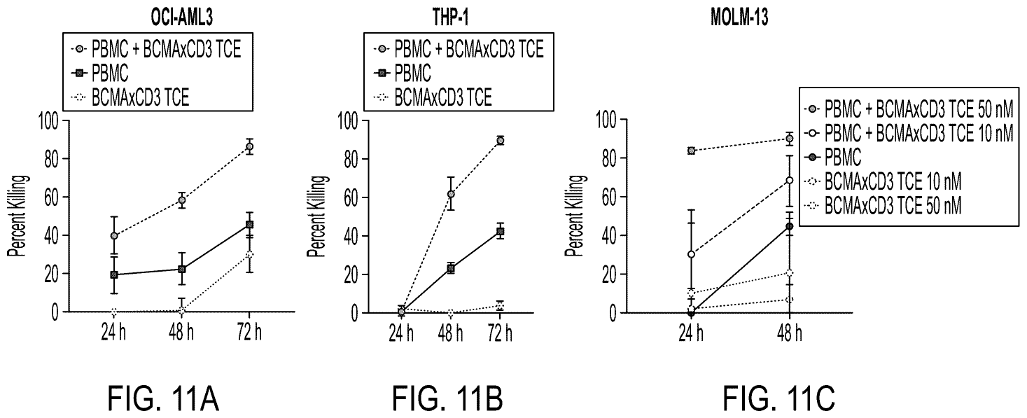

FIG. 11A, FIG. 11B and FIG. 11C shows that BCMAxCD3 bispecific T cell engager (TCE) treatment mediates killing of AML cells in vitro. FIG. 11A: OCI-AML3-luc human AML cells were cultured in the presence of human PBMC at an effector: target ratio of 20:1. BCMA×CD3 TCE (0.4 nM) was added to the media and relative luminescence units (RLU) were measured after 24, 48 and 72 h. Controls included the following conditions: untreated target cells, target cells+PBMC, target cells+TCE. Normalized values (killing percentage based on untreated target cells) are presented (Mean and SEM). FIG. 11B: THP-1-luc human AML cells were cultured in the presence of human PBMC at an effector: target ratio of 10:1. BCMA×CD3 TCE (10 nM) was added to the media and RLU were measured after 24, 48 and 72 h. Controls included the following conditions: untreated target cells, target cells+PBMC, target cells+TCE. Normalized values (killing percentage based on untreated target cells) are presented (Mean and SEM). FIG. 11C: MOLM-13-luc human AML cells were cultured in the presence of human PBMC at an effector: target ratio of 20:1. BCMA×CD3 TCE (10 or 50 nM) was added to the media and relative luminescence units (RLU) were measured after 24 and 48 h. Controls included the following conditions: untreated target cells, target cells+PBMC, target cells+TCE. Normalized values (killing percentage based on untreated target cells) are presented (Mean and SEM).

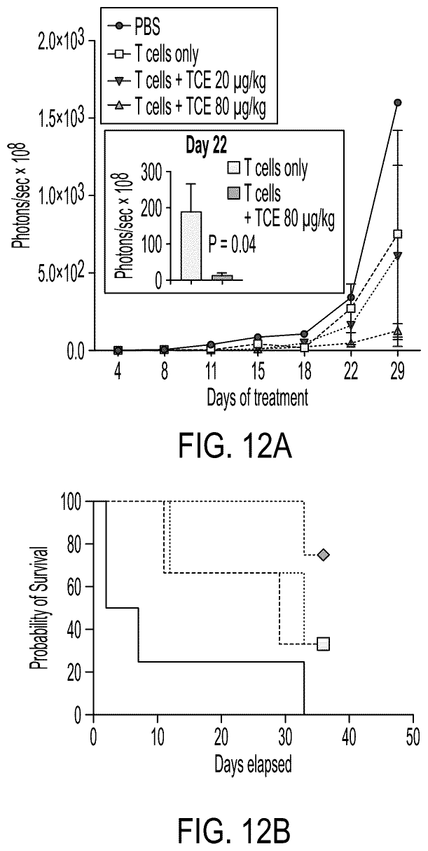

FIG. 12A and FIG. 12B shows that BCMAxCD3 bispecific TCE treatment displays strong antileukemic activity in an AML xenograft model. FIG. 12A. NSG mice received 2.5×106 luciferase-expressing THP-1 cells intravenously. After 10 days engraftment was confirmed, and mice were assigned to the following groups: 1. Placebo (PBS) I.V. on day 0; 2. Human CD8 T cells (1×106) I.V. on day 0; 3. Human CD8 T cells (1×106) I.V. on day 0 followed by BCMAxCD3 bispecific T cell engager (TCE) 20 mg/kg I.V. on days 1, 4, 8, 11, 15, 18, 22; 4. Human CD8 T cells (1×106) I.V. on day 0 followed by TCE 80 mg/kg I.V. on days 1, 4, 8, 11, 15, 18, 22. A: In vivo bioluminescence imaging between days 4 and 29 of treatment. Mean and SEM are presented (n=3-4). FIG. 12B: Kaplan Meier analysis of survival.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for treating an eligible subject with acute myeloid leukemia (AML) including high risk disease features comprising administering to the subject post-consolidation a targeted immunotherapy comprising an immunotherapeutic agent that specifically targets B cell maturation antigen. (BCMA).

2. The method according to claim 1, wherein
a. the eligible subject with AML is a child;
b. the eligible subject with AML is a child less than 2;
c. the eligible subject with AML is a teenage child aged 13-19, inclusive;
d. the eligible subject with AML is an adult;
e. the eligible subject with AML is an adult less than 60;
f. the eligible subject with AML is a young adult, aged 26-50, inclusive;
g. the eligible subject with AML is an adult aged 60-69, inclusive; or
h. the eligible subject with AML is 70 or older.

3. The method according to claim 1, wherein the high risk disease features include biologic features, clinical features, or both.

4. The method according to claim 3, wherein the high risk biological features include antecedent hematological disorders, presence of ≥20% BM blasts in the bone marrow, 0, 1, 2, 3 or more clonal cytogenetic abnormalities, and molecular abnormalities.

5. The method according to claim 4, wherein the antecedent hematological disorder is myelodysplastic syndrome, refractory AML, AML in remission, or mixed-phenotype acute leukemia.

6. The method according to claim 3, wherein the clinical features include comorbidities, measurable residual disease at time of complete remission, refractory to induction chemotherapy; remission after induction therapy, relapsed subject in remission, AML arising out of an antecedent hematologic disorder; and AML in the elderly.

7. The method according to claim 4, wherein the high risk molecular feature is an FLT3 mutation, an NPM1 mutation; an isocitrate dehydrogenase 1 or 2 (IDH1/2) mutation, a RUNX1 mutation, a DNMT3A mutation, a TET2 mutation, a TP53 mutation, or a combination thereof.

8. The method according to claim 7, wherein the FLT3 mutation is an FLT3-ILD mutation.

9. The method according to claim 4, wherein the high risk cytogenetic features include a chromosomal translocation and a monosomy of a somatic chromosome.

10. The method according to claim 9, wherein the translocation is t (8;21) (q22;q22.1); RUNX1-RUNXIT1).

11. The method according to claim 6, wherein high risk features in the elderly include one or more of co-morbidities, a high incidence of cytogenetic abnormalities involving monosomies 5 and 7 and chromosome 17 abnormalities, a high incidence of multiple mutations including TP53, and a high incidence of secondary/therapy-related AML.

12. The method according to claim 11, wherein the comorbidities include one or more of hypertension; diabetes; organ dysfunctions including cardiac, pulmonary and renal abnormalities).

13. The method according to claim 1, wherein the immunotherapy includes
a. an immune cell engager” (“ICE”) selected from a T cell engager, a natural killer (NK) cell engager and cytotoxic/phagocytic cell engagers; and/or
b. an immune checkpoint therapy comprising an immune checkpoint inhibitor, and/or
c. a gamma-secretase inhibitor.

14. The method according to claim 13, wherein
a. the immune cell engager is a bispecific T cell engager (BiTE) comprising a BCMA-targeting molecule connected through a peptide linker to a CD3-targeting molecule, wherein the CD3-targeting molecule activates a specific chain of the CD3 complex associated with the T cell receptor (TCR) complex and participates in assembly of an immunological synapse mediating BCMA-specific cytotoxicity; or
b. the immune cell engager is a CD16 or an NKG2D receptor-directed bispecific NK cell engager that activates NK cells targeting BCMA-specific cytotoxicity; or
c. the immune engager is a chemically linked bispecific molecule engaging the non-ligand binding site of the high-affinity receptor for immunoglobulin G (FcγRI, also known as CD64) selectively expressed by a population of cytotoxic/phagocytic immune cells targeting BCMA antibody-dependent cell-mediated cytotoxicity of AML blast cells.

15. The method according to claim 14, wherein the BCMA-targeting molecule is a BCMA targeting scFv fragment and the CD3-targeting molecule is a CD3 targeting scFv fragment.

16. The method according to claim 14, wherein the CD64 expressing population of cytotoxic/phagocytic immune cells is a population of monocytes, macrophages, dendritic cells or cytokine-activated neutrophils.

17. The method according to claim 13, wherein the T cell immune engager is a BiTE that targets CD3 on T cells and targets BCMA expressed on relapsed/refractory AML blasts.

18. The method according to claim 17, wherein the BiTE is teclistamab, elranatamab, AMG-701, AMG 420 (formerly BI 836909), REGN5458, or TNB-383B.

19. The method according to claim 13, wherein the checkpoint inhibitor is lambrolizumab/pembrolizumab (KEYTRUDA®), nivolumab (OPDIVO®), atezolizumab, TECENTRIQ®, or ipilimumab (YERVOY®).

20. The method according to claim 13, wherein the gamma-secretase inhibitor is LY3039478/JSMD194, dihydroergocristine (DHEC), RO4929097; LY900009; MK-0752; PF-03084014; BMS-986115; GSI-136; AL-101; or Nirogacestat.

21. The method according to claim 1, wherein the immunotherapy includes a BCMA targeted antibody drug conjugate or a BCMA-targeted CAR-T cell therapy described in Table 1.
